(19) United States
(12) Patent Application Publication
(10) Pub. No.: US 2026/0347404 A1
(43) Pub. Date: Nov. 5, 2026

(54) PROLINE-LOCKED STAPLED PEPTIDES AND USES THEREOF

(71) Applicant: President and Fellows of Harvard College, Cambridge, MA (US)

(72) Inventors: Gregory L. VERDINE, Boston, MA (US); Kazuhiro HAYASHI, Cambridge, MA (US)

(21) Appl. No.: 19/307,814

(22) Filed: Aug. 22, 2025

Related U.S. Application Data
(63) Continuation of application No. 18/453,118, filed on Aug. 21, 2023, now abandoned.
Application No. 18/453,118 is a continuation of application No. 17/681,248, filed on Feb. 25, 2022, now abandoned.
Application No. 17/681,248 is a continuation of application No. 15/456,070, filed on Mar. 10, 2017, now abandoned.
Application No. 15/456,070 is a division of application No. 14/431,280, filed on Sep. 26, 2013, now Pat. No. 9,617,309.
(60) Provisional application No. 61/705,950, filed on Sep. 26, 2012.
(60) Provisional application No. 61/789,157, filed on Mar. 15, 2013.

(51) Int. Cl. C07K 7/56; C07D 207/16; C07K 1/113

(52) U.S. Cl. CPC C07K 7/56; C07D 207/16; C07K 1/113

(57) ABSTRACT

The present invention provides a new type of alpha-helix nucleating cross-link (“staple”) formed by olefin metathesis of a proline derivative with an alkenyl side chain and another amino acid derivative with an alkenyl side chain. The proline derivatives as described herein have been found to be strong nucleators of alpha-helix formation. The invention also provides moieties for shielding the free amide N—H's at the N-terminus of an alpha-helix, thereby further stabilizing the helix. The proline derivatives, precursors prior to cross-linking, and the cross-linked peptides are provided as well as methods of using and preparing these compounds and peptides.

### RELATED APPLICATIONS

The present application is a continuation of U.S. patent application Ser. No. 18/453,118, filed Aug. 21, 2023, which is a continuation of Ser. No. 17/681,248, filed Feb. 25, 2022, which is a continuation of U.S. patent application Ser. No. 15/456,070, filed Mar. 10, 2017, which is a divisional of U.S. patent application Ser. No. 14/431,280, filed Mar. 25, 2015, which is the U.S. National Stage of International Application Number PCT/US2013/062004, filed Sep. 26, 2013, which claims the benefit of U.S. Provisional Patent Application No. 61/705,950, filed Sep. 26, 2012, and U.S. Provisional Patent Application No. 61/789,157, filed Mar. 15, 2013, each of which is incorporated herein by reference.

### REFERENCE TO A SEQUENCE LISTING OF THE INVENTION

This application contains a Sequence Listing which has been submitted electronically in XML format. The Sequence Listing XML is incorporated herein by reference. Said XML file, created on Oct. 25, 2023, is named 085298.000322_SL.xml and is 97,419 bytes in size.

### BACKGROUND OF THE INVENTION

The important biological roles that peptides and polypeptides play as hormones, enzyme inhibitors, substrates, neurotransmitters, and neuromediators has led to the widespread use of peptides and peptide mimetics in medicinal chemistry as therapeutic agents. The peptide's bioactive conformation, combining structural elements such as alpha-helices, beta-sheets, turns, and/or loops, is important as it allows for selective biological recognition of receptors or enzymes, thereby influencing cell-cell communication and/or controlling vital cell functions, such as metabolism, immune defense, and reproduction (Babine et al., Chem. Rev. (1997) 97:1359). The alpha-helix is one of the major structural components of peptides. However, alpha-helical peptides have a propensity for unraveling and forming random coils, which are, in most cases, biologically less active, or even inactive, and are highly susceptible to proteolytic degradation.

Many research groups have developed strategies for the design and synthesis of more robust peptides as therapeutics. For example, one strategy has been to incorporate more robust functionalities into the peptide chain while still maintaining the peptide's unique conformation and secondary structure (see, for example, Gante et al., Angew. Chem. Int. Ed Engl. (1994) 33:1699-1720; Liskamp et al., Recl. Tray. Chim. Pavs-Bas (1994) 113:1; Giannis et al., Angew. Chem. Int. Ed. Engl. (1993) 32:1244; P. D. Bailey, Peptide Chemistry, Wiley, New York, 1990, p. 182; and references cited therein). Another approach has been to stabilize the peptide via covalent crosslinks (see, for example, Phelan et al., J. Am. Chem. Soc. (1997) 119:455; Leuc et al., Proc. Nat'l. Acad. Sci. USA (2003) 100:11273; Bracken et al., J. Am. Chem. Soc. (1994) 116:6432; and Yan et al., Bioorg. Med. Chem. (2004) 14:1403). Crosslinking a polypeptide predisposed to have an alpha-helical secondary structure can constrain the polypeptide to its native alpha-helical conformation. The constrained secondary structure may, for example, increase the peptide's resistance to proteolytic cleavage, may increase the peptide's hydrophobicity, may allow for better penetration of the peptide into the target cell (e.g., through an energy-dependent transport mechanism such as pinocytosis), and/or may lead to an improvement in the peptide's biological activity relative to the corresponding uncrosslinked peptide. Therefore, there remains a need and interest in developing new crosslinked alpha-helical polypeptides as therapeutic agents as well as research tools.

### SUMMARY OF THE INVENTION

The present invention provides a new type of alpha-helix nucleating staple formed using an N-terminal proline derivative with an alkenyl or alkynyl side chain (e.g., alpha-allylproline). Although proline is commonly considered to be an alpha-helix disrupting amino acid, it frequently occurs at the N-terminus of alpha-helices. Therefore, proline can be considered to be a helix-nucleating residue. Such a staple using a proline derivative may be formed with any other amino acid with an alkenyl or alkynyl side chain using an olefin metathesis reaction. Proline and the residue preceding it (such as serine, aspartate, and glutamate) have also been found to be good at cloaking the amide N—H's at the beginning of an alpha-helix through the formation of hydrogen bonds and have led to the design of other capping moieties for alpha-helical peptides as described herein. The proline derivative for stapling has been found to be a strong nucleator of alpha-helix formation, and peptides with such a staple may be of use in targeting various extracellular and intracellular targets as well as conferring oral bioavailability on peptides.

In one aspect, the disclosure provides stabilized peptides (e.g., staples and stitched) and methods for increasing the stability of peptides. In some embodiments, the disclosure provides peptides with improved biological properties and methods for improving the biological properties of peptides. The disclosure provides peptides with improved capacity to penetrate cell membranes and/or otherwise get into cells. The disclosure therefore also provides peptides as therapeutic agents and as deliver aids to deliver peptide-drug conjugates intracellularly.

In one aspect, the disclosure provides peptides that are stabilized by stapling the peptide at the N-terminus of an alpha-helix through the introduction of a proline-locked staple. It was surprisingly found that proline could be used to stabilize peptides. The finding was surprising at least because proline is commonly considered an α-helix-disrupting amino acid. In some embodiments, the proline-locked stapled peptide includes a proline at position i that is covalently linked to the alpha-carbon of a second amino acid at position i+3. While alpha-helical peptides are relatively stable once formed, initiation of alpha helix formation is challenging because the attendant conformational ordering is entropically expensive (J. Chem. Phys, 1959, 31, 526-535). As provided herein, introducing a helix staple, such as a proline-locked staple at the N-terminus of an alpha-helical peptide helps with the formation of, and further stabilizes, an alpha-helix. Once a single turn of the α-helix is formed, its downstream propagation can occur spontaneously, provided that helix-disrupting sequences are not present.

In one aspect, the disclosure provides peptides with improved ability to cross cell membranes. An increased ability of peptides to cross the cell membrane is correlated with an increase in the capacity of the peptide to acts as a therapeutic. Peptides often have difficulty crossing (cell) membranes because of the availability of unpaired hydrogen bonds in the peptide (e.g., in the peptide backbone). The disclosure provides methods for minimizing the availability of unpaired hydrogen bonds in a peptide by binding N-terminal amide protons tightly into hydrogen-bonding interactions. As disclosed herein, locating an amino acid with a side chain that can interact with amide protons at the N-terminal side of an alpha helix minimizes the availability of free amide protons. The undesired free N-terminal amides are “masked” thereby minimizing any undesired interactions with other agents (e.g., the cell membrane or components thereof). In some embodiments, the amino acid before the proline is an amino acid with a side chain that can interact with the free amide protons at the beginning of the helix. For instance, the disclosure provides a modified arginine with increased ability to mask N-terminal amide protons by providing additional hydrogen bond acceptor.

In one aspect, the disclosure provides stabilized peptides that nucleate α-helix formation through a proline-locked staple while also binding N-terminal amide protons tightly through hydrogen-bonding interactions. As provided herein, the stabilized peptides with amide proton hydrogen bond acceptors may have a proline at position i that is covalently coupled to an amino acid at position i+3, and a modified arginine residue at position i−1 (as described herein) which interacts with the amide protons of the peptide backbone of the amino acids at position i+1 and i+2. In certain other embodiments, the i−1 position is occupied by a natural amino acid such as serine, aspartate, or glutamate.

The proline-locked stapled peptides provided herein are strong nucleators of α-helix formation, as shown by the high helicity of peptides bearing the proline-lock feature. In addition, the peptides provided herein, through masking the N-terminal amide protons, further enhance the ability of the peptides to cross cell membranes. Thus, the Pro-locked stapled peptides provided herein may be used in targeting previously “undruggable” intracellular therapeutic targets.

The details of one or more embodiments of the invention are set forth in the Detailed Description of Certain Embodiments, as described below. Other features, objects, and advantages of the invention will be apparent from the Definitions, Examples, Figures, and Claims.

### BRIEF DESCRIPTION OF THE DRAWINGS

FIG. 1 provides an example of a proline-locked stapled peptide 4 (SEQ ID NO: 2).

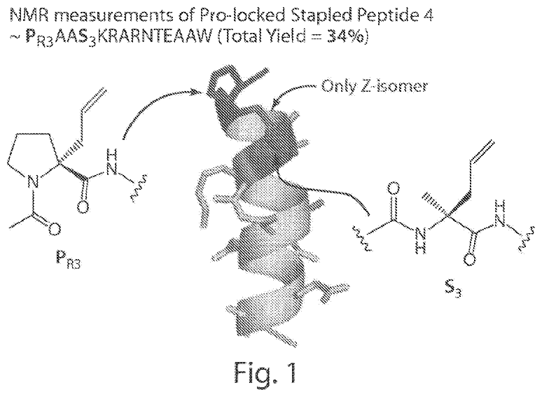

FIG. 2 shows two peptides with partial basic region of GCN4.

FIG. 3 shows a LC/MS chromatogram of the olefin-metathesis reaction between P_{R3} and S₃.

FIG. 4 shows the CD spectra of peptides 1 and 2 at 20° C.

FIG. 5 shows the variable-temperature CD spectra of peptide 2 in 50 mM sodium phosphate solution (pH=8.0).

FIG. 6 shows an olefin-metathesis resulting in a stapled peptide.

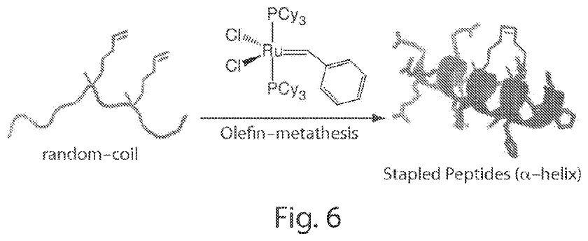

FIG. 7 shows a capping strategy for passive membrane diffusion with a proline-locked stapled peptide.

FIG. 8 shows a capping strategy for passive membrane diffusion with asparagine and an asparagine surrogate.

FIG. 9 shows a GCN4-DNA complex and the basic and coiled-coil regions of GCN4.

FIG. 10 shows unnatural amino acids used to generate proline-locked stapled peptides.

FIG. 11A. and FIG. 11 B show a synthesis scheme for P_{R3}.

FIG. 12 shows a synthesis scheme for P_{SO3}.

FIG. 13 shows examples of proline-locked stapled peptides (SEQ ID NOS 1, 5, 6, 2, 7-12, 40, 13 and 14, respectively, in order of appearance).

FIG. 14 shows examples of proline-locked stapled and unstapled peptides (SEQ ID NOS 15-21, respectively, in order of appearance).

FIG. 15 shows an example of olefin-metathesis by Grubbs-catalysis.

FIG. 16 shows a LC/MS chromatogram of the olefin-metathesis reaction by Grubbs-catalysis of peptide “4)” (SEQ ID NO:2).

FIG. 17 shows CD spectra of proline-locked stapled peptides.

FIG. 18 shows CD spectra of proline-locked stapled peptides.

FIG. 19 shows CD spectra of selected proline-locked stapled peptides in various solutions.

FIG. 20 shows CD spectra of selected proline-locked stapled peptides at various temperatures.

FIG. 21 shows CD spectra of proline-locked stapled peptides based on the full-length GCN basic region (SEQ ID NOS 22, 23 and 41, respectively, in order of appearance).

FIG. 22 shows CD spectra of proline-locked stapled peptides at various temperatures. “20 Mix” refers to E and Z isomers mixture of peptide 20.

FIG. 23 shows the CD spectra of proline-locked stapled peptides (24 mer).

FIG. 24 shows the ability of proline-locked stapled peptides 17 and 18 (SEQ ID NO: 23) to penetrate cells at the concentration of 0.1 μM.

FIG. 25 shows the ability of proline-locked stapled peptides 17 and 18 to penetrate cells at the concentration of 1 μM.

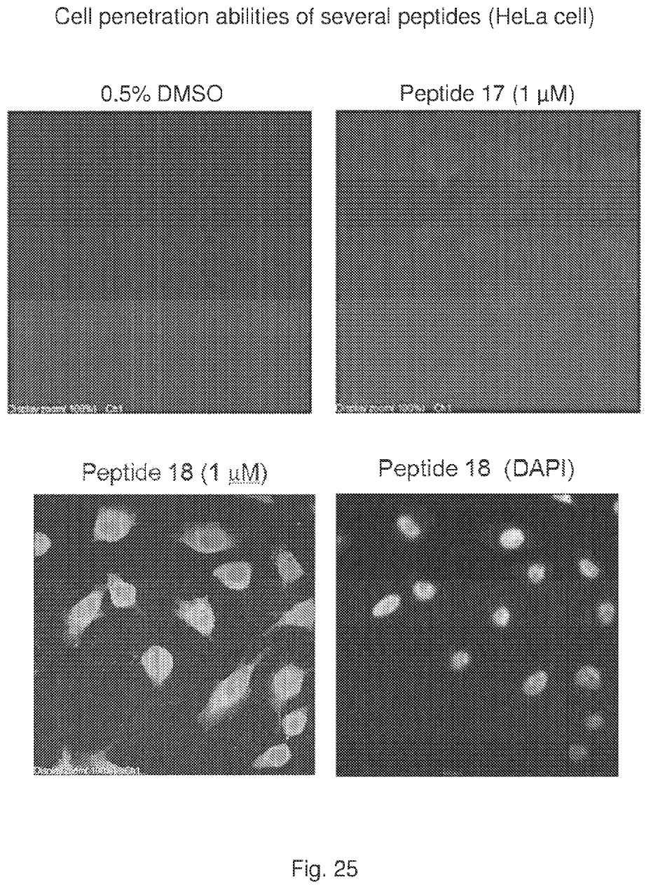

FIGS. 26 A-26D. show investigation of the endocytosis mechanism of the peptides. The peptides were labeled with FITC. FIG. 26 A shows flow cytometry of peptide 17 and peptide 18. FIG. 26 B shows flow cytometry of peptide 18 at different temperatures. FIG. 26 C shows CD spectra of peptide 18 at different temperatures. FIG. 26 D shows flow cytometry of peptide 18 in the presence of NaN₃+2-deoxy-D-glucose (2-DG), These data indicate that the internalization of peptide 18 is ATP-dependent.

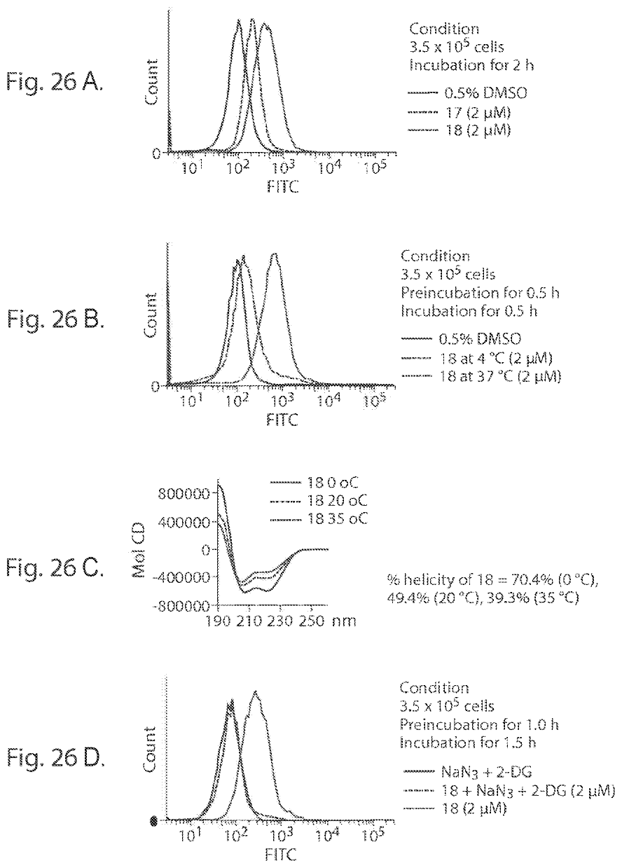

FIG. 27 shows analysis of 1H NMR and NOESY spectra of peptide 4 (SEQ ID NO: 2). The crosspeaks indicate alpha-helix conformation of the peptides. dαN(i, i+3) indicates the interaction between an amide N—H at i position and an alpha proton at i+3 position. dαN(i, i+4) indicates the interaction between an amide N—H at i position and an alpha proton at i+4 position. The coupling constant below 4 indicates alpha-helix or 310-helix. The coupling constant below 7 means the existence of a helical structure including random coil. The residues adopt helical structure at N and T because dNN(i, i+1) interaction was observed in these residues. The 13 crosspeaks observed in NOESY spectra of peptide 4 indicate alpha-helix conformation.

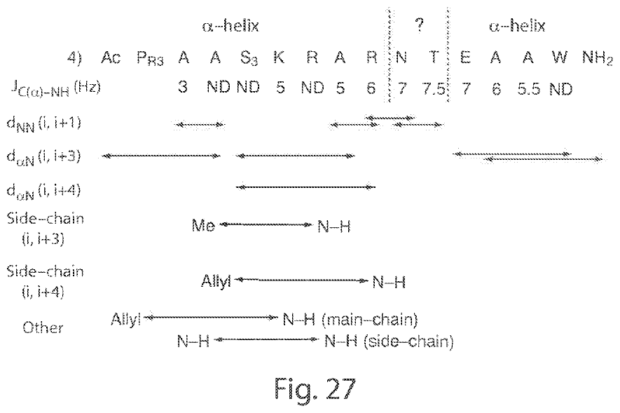

FIG. 28 shows NMR measurements of peptide 4 (SEQ ID NO: 2). The coupling constant between two olefinic protons is 11 Hz, which means the olefin in peptide 4 is of the Z conformation. High % NOE value was observed between two olefinic protons (49% and 77%). These values indicate the Z-conformation of the olefin in peptide 4.

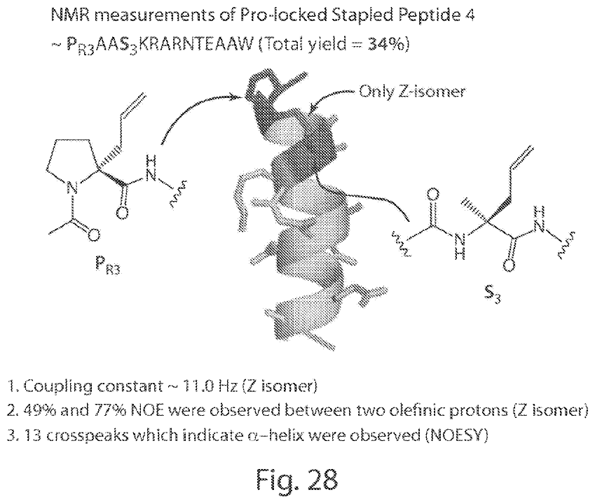

FIGS. 29A. and B. show CD spectra and NMR of Pro-locked Stapled peptides (5 mer) (SEQ ID NOS 24, 25, 28 and 58, respectively, in order of appearance). FIG. 29A. shows shows CD spectra of peptides 21-24. FIG. 29 B. shows NMR of peptide 22 isomers.

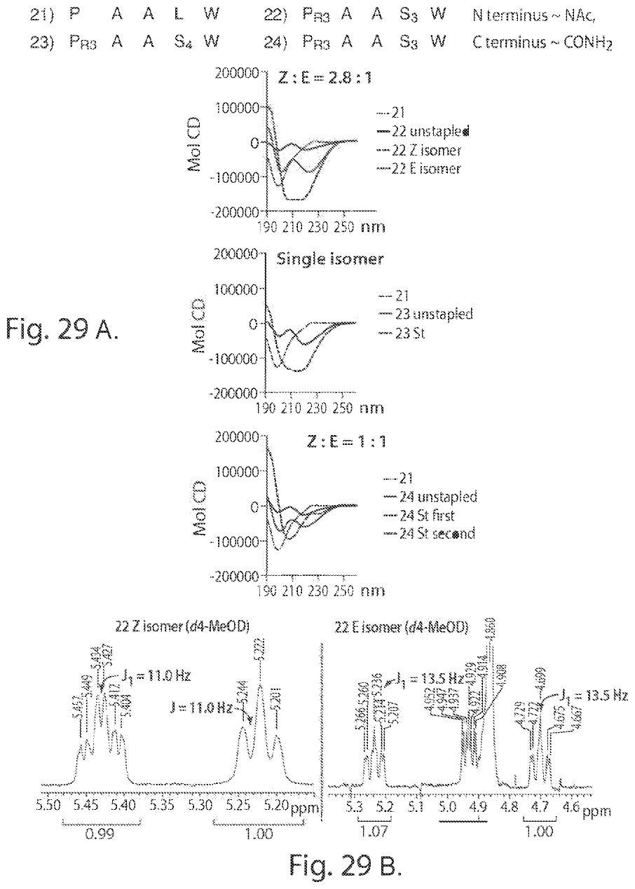

FIG. 30 shows CD spectra of selected stapled peptides (GCN 14 mer) (SEQ ID NOS 1, 2, 41, 13, 14, 42 and 43, respectively, in order of appearance).

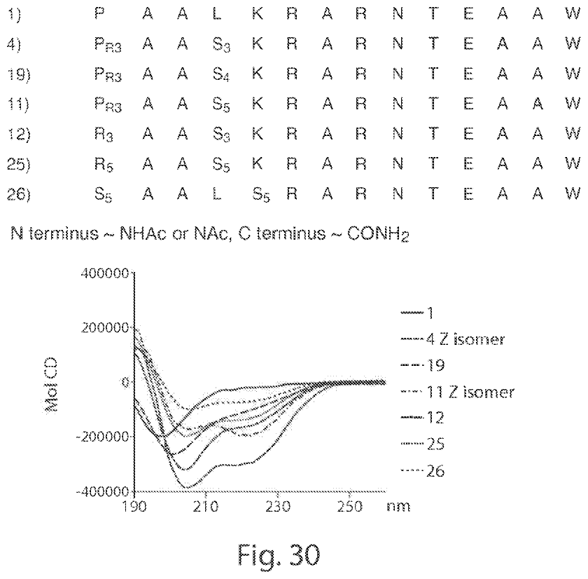

FIG. 31 shows proline stapled peptides against trypsin proteolysis (SEQ ID NOS 2, 27 and 28, respectively, in order of appearance.

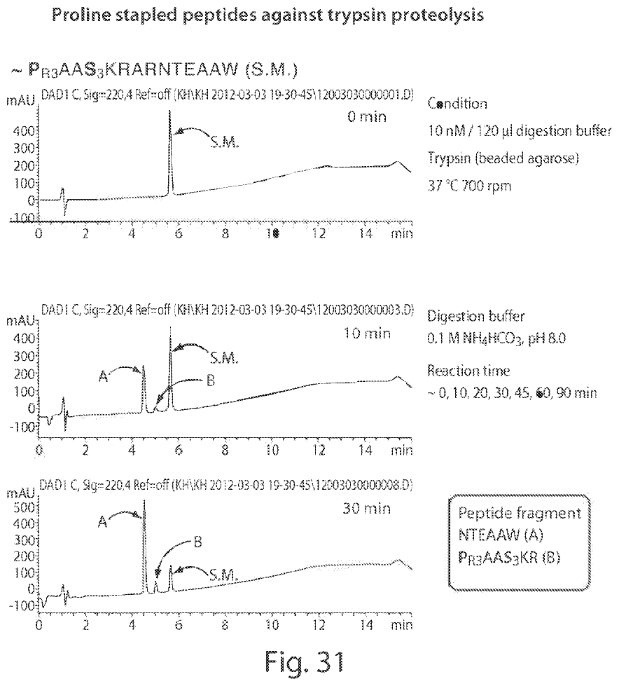

FIG. 32 shows stability of proline stapled peptides against trypsin proteolysis (SEQ ID NOS 1, 5 and 2, respectively, in order of appearance).

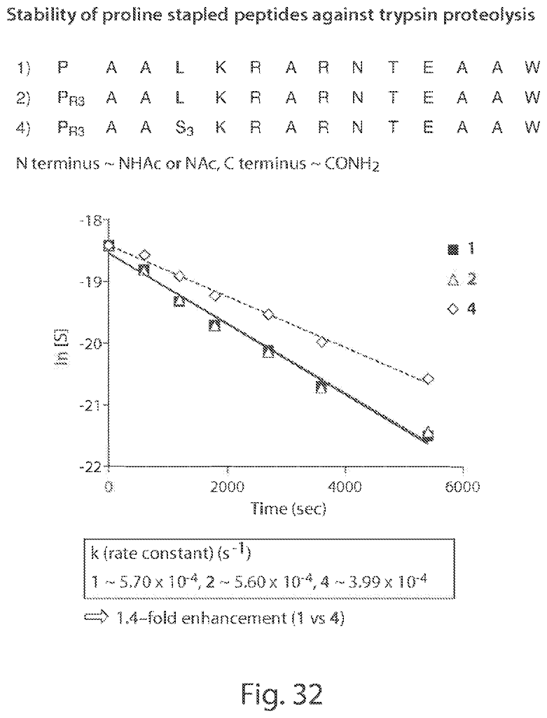

FIG. 33A. and FIG. 33B show CD spectra of exemplified stapled peptides (SEQ ID NOS 1, 2 and 4448, respectively, in order of appearance).

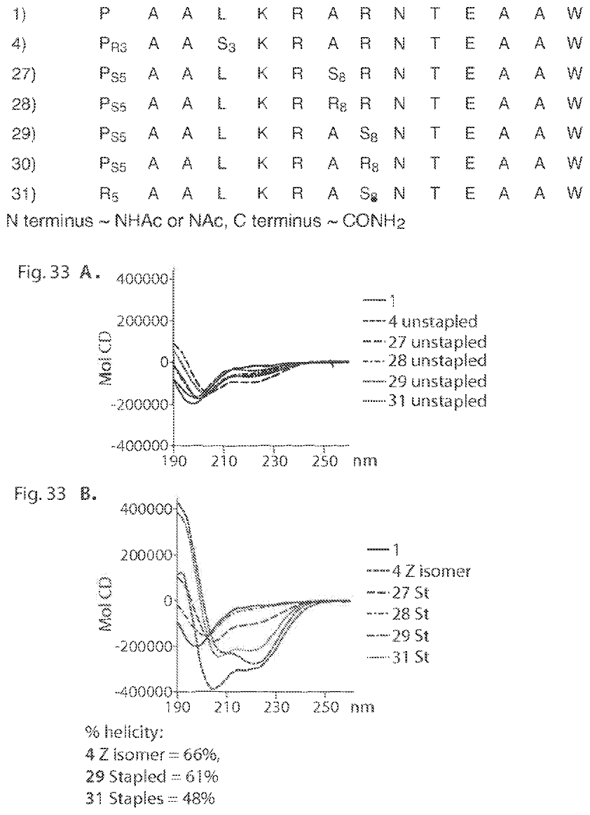

FIG. 34 shows melting curve of exemplified pro-locked stapled peptides (i, i+7).

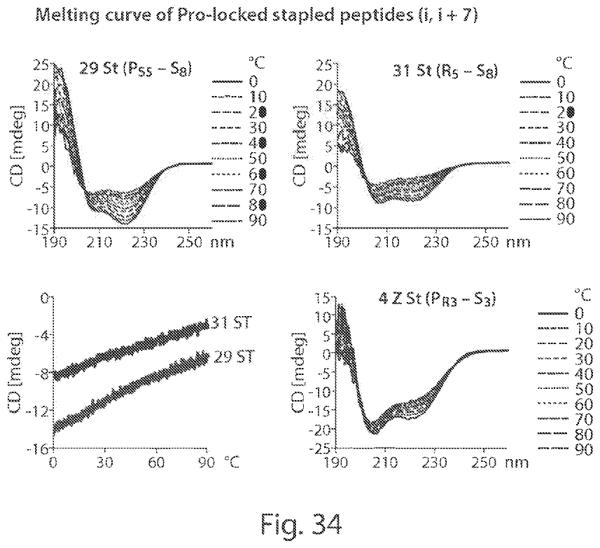

FIG. 35 shows CD spectra of exemplified stapled peptides (SEQ ID NOS 1, 2 and 46 and 49-51, respectively, in order of appearance).

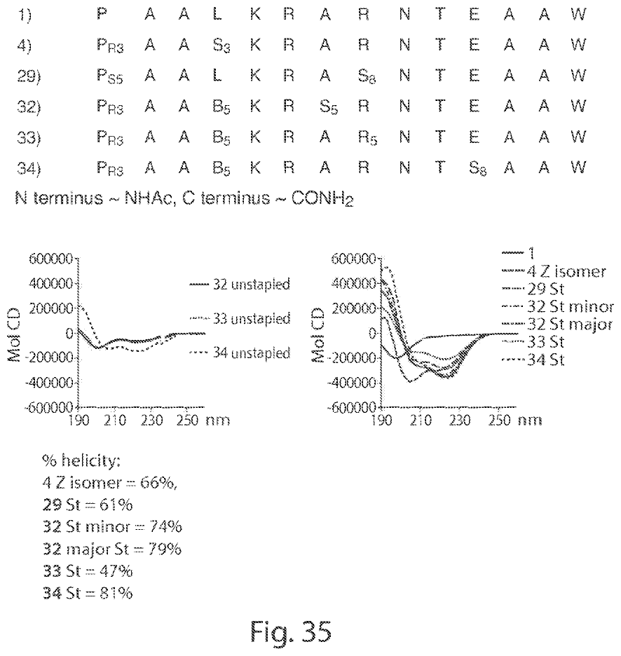

FIG. 36 shows exemplified designed capping molecules.

FIG. 37 A. and FIG. 37B show CD spectra of exemplified stapled peptides (SEQ ID NOS 52-57, respectively, in order of appearance).

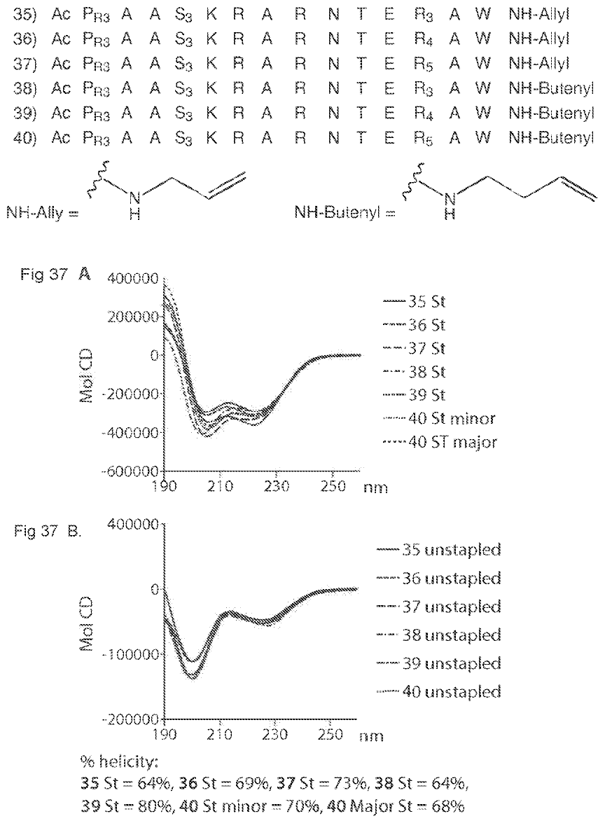

### DEFINITIONS

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds and polypeptides described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H. Tables of Resolving Agents and Optical Resolutions p. 268 (E. L. Eliel, Ed., Univ. of Notre Dame Press, Notre Dame, IN 1972). The invention additionally encompasses compounds and polypeptides described herein as individual isomers substantially free of other isomers, and alternatively, as mixtures of various isomers.

When a range of values is listed, it is intended to encompass each value and sub-range within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

As used herein, substituent names which end in the suffix “-ene” refer to a biradical derived from the removal of an additional hydrogen atom from monoradical group as defined herein. Thus, for example, the monoradical alkyl, as defined herein, is the biradical alkylene upon removal of an additional hydrogen atom. Likewise, alkenyl is alkenylene; alkynyl is alkynylene; heteroalkyl is heteroalkylene; heteroalkenyl is heteroalkenylene; heteroalkynyl is heteroalkynylene; carbocyclyl is carbocyclylene; heterocyclyl is heterocyclylene; aryl is arylene; and heteroaryl is heteroarylene.

The term “aliphatic,” as used herein, refers to alkyl, alkenyl, alkynyl, and carbocyclic groups. Likewise, the term “heteroaliphatic” as used herein, refers to heteroalkyl, heteroalkenyl, heteroalkynyl, and heterocyclic groups.

As used herein, “alkyl” refers to a radical of a straight-chain or branched saturated hydrocarbon group having from 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₄ alkyl”). Examples of C₁₋₆ alkyl groups include methyl (C₁), ethyl (C₂), n-propyl (C₃), isopropyl (C₃), n-butyl (C₄), tert-butyl (C₄) sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Unless otherwise specified, each instance of an alkyl group is independently unsubstituted (an “unsubstituted alkyl”) or substituted (a “substituted alkyl”) with one or more substituents. In certain embodiments, the alkyl group is an unsubstituted C₁₋₁₀ alkyl (e.g., —CH₃). In certain embodiments, the alkyl group is a substituted C₁₋₁₀ alkyl.

As used herein, “haloalkyl” is a substituted alkyl group as defined herein wherein one or more of the hydrogen atoms are independently replaced by a halogen, e.g., fluoro, bromo, chloro, or iodo. “Perhaloalkyl” is a subset of haloalkyl, and refers to an alkyl group wherein all of the hydrogen atoms are independently replaced by a halogen, e.g., fluoro, bromo, chloro, or iodo. In some embodiments, the haloalkyl moiety has 1 to 8 carbon atoms (“C₁₋₈ haloalkyl”). In some embodiments, the haloalkyl moiety has 1 to 6 carbon atoms (“C₁₋₆ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). In some embodiments, all of the haloalkyl hydrogen atoms are replaced with fluoro to provide a perfluoroalkyl group. In some embodiments, all of the haloalkyl hydrogen atoms are replaced with chloro to provide a “perchloroalkyl” group. Examples of haloalkyl groups include —CF₃, —CF₂CF₃, —CF₂CF₂CF₃, —CCl₃, —CFCl₂, —CF₂Cl, and the like.

As used herein, “heteroalkyl” refers to an alkyl group as defined herein which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkyl group refers to a saturated group having from 1 to 10 carbon atoms and 1, 2, 3, or 4 heteroatoms within the parent chain (“heteroC₁₋₁₀ alkyl”). In some embodiments, a heteroalkyl group is a saturated group having 1 to 9 carbon atoms and 1, 2, 3, or 4 heteroatoms within the parent chain (“heteroC₁₋₉ alkyl”). In some embodiments, a heteroalkyl group is a saturated group having 1 to 8 carbon atoms and 1, 2, 3, or 4 heteroatoms within the parent chain (“heteroC₁₋₈ alkyl”). In some embodiments, a heteroalkyl group is a saturated group having 1 to 7 carbon atoms and 1, 2, 3, or 4 heteroatoms within the parent chain (“heteroC₁₋₇ alkyl”). In some embodiments, a heteroalkyl group is a saturated group having 1 to 6 carbon atoms and 1, 2, or 3 heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and lor 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₀ alkyl. In certain embodiments, the heteroalkyl group is a substituted heteroC₁₋₁₀ alkyl.

As used herein, “alkenyl” refers to a radical of a straight-chain or branched hydrocarbon group having from 2 to 10 carbon atoms and one or more double bonds (e.g., 1, 2, 3, or 4 double bonds) and no triple bonds. In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₂₋₁₀ alkenyl. In certain embodiments, the alkenyl group is a substituted C₂₋₁₀ alkenyl.

As used herein, “heteroalkenyl” refers to an alkenyl group as defined herein which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkenyl group refers to a group having from 2 to 10 carbon atoms, at least one double bond, and 1, 2, 3, or 4 heteroatoms within the parent chain (“heteroC₂₋₁₀ alkenyl”). In some embodiments, a heteroalkenyl group has 2 to 9 carbon atoms at least one double bond, and 1, 2, 3, or 4 heteroatoms within the parent chain (“heteroC₂₋₉ alkenyl”). In some embodiments, a heteroalkenyl group has 2 to 8 carbon atoms, at least one double bond, and 1, 2, 3, or 4 heteroatoms within the parent chain (“heteroC₂₋₈ alkenyl”). In some embodiments, a heteroalkenyl group has 2 to 7 carbon atoms, at least one double bond, and 1, 2, 3, or 4 heteroatoms within the parent chain (“heteroC₂₋₇ alkenyl”). In some embodiments, a heteroalkenyl group has 2 to 6 carbon atoms, at least one double bond, and 1, 2, or 3 heteroatoms within the parent chain (“heteroC₂₋₆ alkenyl”). In some embodiments, a heteroalkenyl group has 2 to 5 carbon atoms, at least one double bond, and 1 or 2 heteroatoms within the parent chain (“heteroC₂₋₅ alkenyl”). In some embodiments, a heteroalkenyl group has 2 to 4 carbon atoms, at least one double bond, and lor 2 heteroatoms within the parent chain (“heteroC₂₋₄ alkenyl”). In some embodiments, a heteroalkenyl group has 2 to 3 carbon atoms, at least one double bond, and 1 heteroatom within the parent chain (“heteroC₂₋₃ alkenyl”). In some embodiments, a heteroalkenyl group has 2 to 6 carbon atoms, at least one double bond, and 1 or 2 heteroatoms within the parent chain (“heteroC₂₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₁₀ alkenyl. In certain embodiments, the heteroalkenyl group is a substituted heteroC₂₋₁₀ alkenyl.

As used herein, “alkynyl” refers to a radical of a straight-chain or branched hydrocarbon group having from 2 to 10 carbon atoms and one or more triple bonds (e.g., 1, 2, 3, or 4 triple bonds) and optionally one or more double bonds (e.g., 1, 2, 3, or 4 double bonds) (“C₂₋₁₀ alkynyl”). An alkynyl group that has one or more triple bonds and one or more double bonds is also referred to as an “ene-yene” group. In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₂₋₁₀ alkynyl. In certain embodiments, the alkynyl group is a substituted C₂₋₁₀ alkynyl.

As used herein, “heteroalkynyl” refers to an alkynyl group as defined herein which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkynyl group refers to a group having from 2 to 10 carbon atoms, at least one triple bond, and 1, 2, 3, or 4 heteroatoms within the parent chain (“heteroC₂₋₁₀ alkynyl”). In some embodiments, a heteroalkynyl group has 2 to 9 carbon atoms, at least one triple bond, and 1, 2, 3, or 4 heteroatoms within the parent chain (“heteroC₂₋₉ alkynyl”). In some embodiments, a heteroalkynyl group has 2 to 8 carbon atoms, at least one triple bond, and 1, 2, 3, or 4 heteroatoms within the parent chain (“heteroC₂₋₈ alkynyl”). In some embodiments, a heteroalkynyl group has 2 to 7 carbon atoms, at least one triple bond, and 1, 2, 3, or 4 heteroatoms within the parent chain (“heteroC₂₋₇ alkynyl”). In some embodiments, a heteroalkynyl group has 2 to 6 carbon atoms, at least one triple bond, and 1, 2, or 3 heteroatoms within the parent chain (“heteroC₂₋₆ alkynyl”). In some embodiments, a heteroalkynyl group has 2 to 5 carbon atoms, at least one triple bond, and 1 or 2 heteroatoms within the parent chain (“heteroC₂₋₅ alkynyl”). In some embodiments, a heteroalkynyl group has 2 to 4 carbon atoms, at least one triple bond, and lor 2 heteroatoms within the parent chain (“heteroC₂₋₄ alkynyl”). In some embodiments, a heteroalkynyl group has 2 to 3 carbon atoms, at least one triple bond, and 1 heteroatom within the parent chain (“heteroC₂₋₃ alkynyl”). In some embodiments, a heteroalkynyl group has 2 to 6 carbon atoms, at least one triple bond, and 1 or 2 heteroatoms within the parent chain (“heteroC₂₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂₋₁₀ alkynyl. In certain embodiments, the heteroalkynyl group is a substituted heteroC₂₋₁₀ alkynyl.

As used herein, “carbocyclyl” or “carbocyclic” refers to a radical of a non-aromatic cyclic hydrocarbon group having from 3 to 10 ring carbon atoms (“C₃₋₁₀ carbocyclyl”) and zero heteroatoms in the non-aromatic ring system. In some embodiments, a carbocyclyl group has 3 to 8 ring carbon atoms (“C₃₋₈ carbocyclyl”). In some embodiments, a carbocyclyl group has 3 to 6 ring carbon atoms (“C₃₋₆ carbocyclyl”). In some embodiments, a carbocyclyl group has 3 to 6 ring carbon atoms (“C₃₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₀ carbocyclyl. In certain embodiments, the carbocyclyl group is a substituted C₃₋₁₀ carbocyclyl.

In some embodiments, “carbocyclyl” is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₄ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₀ cycloalkyl. In certain embodiments, the cycloalkyl group is a substituted C₃₋₁₀ cycloalkyl.

As used herein, “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include, without limitation, azirdinyl, oxiranyl, thiorenyl. Exemplary 4-membered heterocyclyl groups containing 1 heteroatom include, without limitation, azetidinyl, oxetanyl and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, dioxanyl. Exemplary 6-membered heterocyclyl groups containing 2 heteroatoms include, without limitation, triazinanyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo-[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

As used herein, “aryl” refers to a radical of a monocyclic or polycyclic (e.g., bicyclic or tricyclic) 4n+2 aromatic ring system (e.g., having 6, 10, or 14 a electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

“Aralkyl” is a subset of “alkyl” and refers to an alkyl group, as defined herein, substituted by an aryl group, as defined herein, wherein the point of attachment is on the alkyl moiety.

As used herein, “heteroaryl” refers to a radical of a 5-14 membered monocyclic or polycyclic (e.g., bicyclic or tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include, without limitation, pyrrolyl, furanyl and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl and phenazinyl.

“Heteroaralkyl” is a subset of “alkyl” and refers to an alkyl group, as defined herein, substituted by a heteroaryl group, as defined herein, wherein the point of attachment is on the alkyl moiety.

As used herein, the term “partially unsaturated” refers to a group that includes at least one double or triple bond. The term “partially unsaturated” is intended to encompass rings having multiple sites of unsaturation, but is not intended to include aromatic groups (e.g., aryl or heteroaryl moieties) as herein defined.

As used herein, the term “saturated” refers to a group that does not contain a double or triple bond, i.e., contains all single bonds.

As understood from the above, alkyl, alkenyl, alkynyl, heteroalkyl, heteroalkenyl, heteroalkynyl, carbocyclyl, heterocyclyl, aryl, and heteroaryl groups, as defined herein, are, in certain embodiments, optionally substituted. Optionally substituted refers to a group which may be substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb}), —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)₂R^{aa}, —OP(═O)₂R^{aa}, —P(═O)(R^{aa})₂, —OP(═O)(R^{aa})₂, —OP(═O)(OR^{aa})₂, —P(═O)₂N(R^{bb})₂, —OP(═O)₂N(R^{bb})₂, —P(═O)(NR^{bb}), —OP(═O)(NR^{bb})₂, —NR^{bb}P(═O)(OR^{aa})₂, —NR^{bb}P(═O)(NR^{bb})₂, —P(R^{cc})₂, —P(R^{cc})—, —OP(R^{cc})₂, —OP(R^{cc})₃, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, C₃₋₁₄ carbocyclyl, 3-14 membered heterocyclyl, C₆₋₁₄ aryl, and 5-14 membered heteroaryl, wherein each alkyl, alkenyl, alkynyl, carbocyclyl, heterocyclyl, aryl, and heteroaryl is independently substituted with 0, 1, 2, 3, 4, or 5 R^{dd} groups;
or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}*S(═O)₂R^{aa}, ═NR^{bb}, or ═NOR^{cc};
each instance of R^{aa} is, independently, selected from C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{aa})₂, —P(═O)(NR^{cc})₂, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{cc} is, independently, selected from hydrogen. C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{cc}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)₂R^{ee}, —P(═O)(R^{ee})₂, —OP(═O)(R^{ee})₂, —OP(═O)(OR^{ee}), C₁₋₆ alkyl, C₁₋₆ perhaloalkyl, C₂₋₆ alkenyl, C₂₋₆ alkynyl, C₃₋₁₀ carbocyclyl, 3-10 membered heterocyclyl, C₆₋₄₀ aryl, 5-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S;
each instance of R^{ee} is, independently, selected from C₁₋₆ alkyl, C₁₋₆ perhaloalkyl, C₂₋₆ alkenyl, C₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups;
each instance of R^{f} is, independently, selected from hydrogen, C₁₋₆ alkyl, C₁₋₆ perhaloalkyl, C₂₋₆ alkenyl, C₂₋₆ alkynyl, C₃₋₁₀ carbocyclyl, 3-10 membered heterocyclyl, C₆₋₁₀ aryl and 5-10 membered heteroaryl, or two R^{f} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{gg} groups; and
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃ —C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)₂(C₁₋₆ alkyl), —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

As used herein, the term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{aa})₂, —OP(R^{cc})₃, —OP(═O)₂R^{aa}, —OP(═O)(R^{aa})₂, —OP(═O)(OR^{cc})₂, —OP(═O)₂N(R^{bb})₂, and —OP(═O)(NR^{bb})₂, wherein R^{aa}, R^{bb}, and R^{cc} are as defined herein.

As used herein, the term “thiol” or “thio” refers to the group —SH. The term “substituted thiol” or “substituted thio,” by extension, refers to a thiol group wherein the sulfur atom directly attached to the parent molecule is substituted with a group other than hydrogen, and includes groups selected from —SR^{aa}, —S═SR^{cc}, —SC(═S)SR^{aa}, —SC(═O)SR^{aa}, —SC(═O)OR^{aa}, and —SC(═O)R^{aa}, wherein R^{aa} and R^{cc} are as defined herein.

As used herein, the term, “amino” refers to the group —NH₂. The term “substituted amino,” by extension, refers to a monosubstituted amino, a disubstituted amino, or a trisubstituted amino, as defined herein.

As used herein, the term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb}), —NHSO₂R^{aa}, —NHP(═O)(OR^{cc})₂, and —NHP(═O)(NR^{bb})₂, wherein R^{aa}, R^{bb}, and R^{cc} are as defined herein, and wherein R^{bb} of the group —NH(R^{bb}) is not hydrogen.

As used herein, the term “disubstituted amino” refers to an amino group wherein the nitrogen atom directly attached to the parent molecule is substituted with two groups other than hydrogen, and includes groups selected from —N(R^{bb})₂, —NR^{bb}C(═O)R^{aa}, —NR^{bb}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}P(═O)(NR^{bb})₂, wherein R^{aa}, R^{bb}, and R^{cc} are as defined herein, with the proviso that the nitrogen atom directly attached to the parent molecule is not substituted with hydrogen.

As used herein, the term “trisubstituted amino” or a “quaternary amino salt” or a “quaternary salt” refers to a nitrogen atom covalently attached to four groups such that the nitrogen is cationic, wherein the cationic nitrogen atom is further complexed with an anionic counterion, e.g., such as groups of the Formula —N(R^{bb})₃⁺X⁻ and —N(R^{bb})₂—⁺X⁻, wherein R^{bb} and X⁻ are as defined herein.

As used herein, a “counterion” or “anionic counterion” is a negatively charged group associated with a cationic quaternary amino group in order to maintain electronic neutrality. Exemplary counterions include halide ions (e.g., F⁻, Cl⁻, Br⁻, I⁻), NO₃⁻, ClO₄⁻, OH⁻, H₂PO₄⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), and carboxylate ions (e.g., acetate, ethanoate, propanoate, benzoate, glycerate, lactate, tartrate, glycolate, and the like).

As used herein, the term “sulfonyl” refers to a group selected from —SO₂N(R^{bb})₂, —SO₂R^{aa}, and —SO₂OR^{aa}, wherein R^{aa} and R^{bb} are as defined herein.

As used herein, the term “sulfinyl” refers to the group —S(═O)R^{aa}, wherein R^{aa} is as defined herein.

As used herein, the term “acyl” refers a group wherein the carbon directly attached to the parent molecule is sp² hybridized, and is substituted with an oxygen, nitrogen or sulfur atom, e.g., a group selected from ketones (—C(═O)R^{aa}), carboxylic acids (—CO₂H), aldehydes (—CHO), esters (—CO₂R^{aa}), thioesters (—C(═O)SR^{aa}, —C(═S)SR^{aa}), amides (—C(═O)N(R^{bb})₂, —C(═O)NR^{bb}SO₂R^{aa}) thioamides (—C(═S)N(R^{bb})₂), and imines (—C(═NR^{bb})R^{aa}, —C(═NR^{bb})OR^{aa}), —C(═NR^{bb})N(R^{bb})₂), wherein R^{aa} and R^{bb} are as defined herein.

As used herein, the term “azido” refers to a group of the formula —N₃.

As used herein, the term “cyano” refers to a group of the formula —CN.

As used herein, the term “isocyano” refers to a group of the formula —NC.

As used herein, the term “nitro” refers to a group of the formula —NO₂.

As used herein, the term “halo” or “halogen” refers to fluorine (fluoro, —F), chlorine (chloro, —Cl), bromine (bromo, —Br), or iodine (iodo, —I).

As used herein, the term “oxo” refers to a group of the formula ═O.

As used herein, the term “thiooxo” refers to a group of the formula ═S.

As used herein, the term “imino” refers to a group of the formula ═N(R^{b}).

As used herein, the term “silyl” refers to the group —Si(R^{aa})₃, wherein R^{aa} is as defined herein.

Nitrogen atoms can be substituted or unsubstituted as valency permits, and include primary, secondary, tertiary, and quaternary nitrogen atoms. Exemplary nitrogen atom substitutents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups attached to a nitrogen atom are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{d} groups, and wherein R^{aa}, R^{bb}, R^{cc} and R^{dd} are as defined above.

In certain embodiments, the substituent present on the nitrogen atom is an amino protecting group (also referred to herein as a “nitrogen protecting group”). Amino protecting groups include, but are not limited to, —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Amino protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, amino protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide and o-(benzoyloxymethyl)benzamide.

Amino protecting groups such as carbamate groups (e.g., —C(═O)OR^{aa}) include, but are not limited to, methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, I-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Amino protecting groups such as sulfonamide groups (e.g., —S(═O)₂R^{aa}) include, but are not limited to, p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), 0-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other amino protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, the substituent present on an oxygen atom is a hydroxyl protecting group (also referred to herein as an “oxygen protecting group”). Hydroxyl protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃, —P(═O)₂R^{aa}, —P(═O)(R^{aa})₂, —P(═O)(OR^{cc})₂, —P(═O)₂N(R^{bb})₂, and —P(═O)(NR^{bb})₂, wherein R^{aa}, R^{bb}, and R^{cc} are as defined herein. Hydroxyl protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary hydroxyl protecting groups include, but are not limited to, methyl, methoxylmethyl (MOM), methylthiomethyl (MTM), t-butylthiomethyl, (phenyldimethylsilvl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, 1-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,0-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

A “thiol protecting group” is well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Examples of protected thiol groups further include, but are not limited to, thioesters, carbonates, sulfonates allyl thioethers, thioethers, silyl thioethers, alkyl thioethers, arylalkyl thioethers, and alkyloxyalkyl thioethers. Examples of ester groups include formates, acetates, proprionates, pentanoates, crotonates, and benzoates. Specific examples of ester groups include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetate), crotonate, 4-methoxy-crotonate, benzoate, p-benzylbenzoate, 2,4,6-trimethylbenzoate. Examples of carbonates include 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl carbonate. Examples of silyl groups include trimethylsilyl, triethylsilyl, t-butyldimethylsilyl, t-butyldiphenylsilyl, triisopropylsilyl ether, and other trialkylsilyl ethers. Examples of alkyl groups include methyl, benzyl, p-methoxybenzyl, 3,4-dimethoxybenzyl, trityl, t-butyl, and allyl ether, or derivatives thereof. Examples of arylalkyl groups include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, 2- and 4-picolyl ethers.

The term “amino acid” refers to a molecule containing both an amino group and a carboxyl group. Amino acids include alpha-amino acids and beta-amino acids, the structures of which are depicted below. In certain embodiments, the amino acid is an alpha amino acid. In certain embodiments, the amino acid is an unnatural amino acid. In certain embodiments, the amino acid is a natural amino acid. In certain embodiments, the amino acid is an unnatural amino acid.

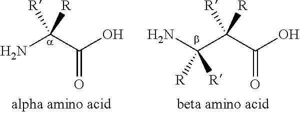

Exemplary amino acids include, without limitation, natural alpha amino acids such as D- and L-isomers of the 20 common naturally occurring alpha amino acids found in peptides, u peptides (e.g., A, R, N, C, D, Q, E, G, H, I, L, K, M, F, P, S, T, W, Y, V, as provided in Table 1 depicted below), unnatural alpha-amino acids (as depicted in Tables 2 and 3 below), natural beta-amino acids (e.g. beta-alanine), and unnatural beta-amino acids.

Amino acids used in the construction of peptides of the present invention may be prepared by organic synthesis, or obtained by other routes, such as, for example, degradation of or isolation from a natural source. In certain embodiments of the present invention, the formula —[X_{AA}]— or -[G]- corresponds to the natural and/or unnatural amino acids having the following formulae:

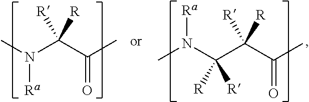

wherein R and R′ correspond a suitable amino acid side chain, as defined below and herein, and R^{a} is as defined below and herein.

TABLE 1

| Exemplary natural | Amino acid side chains | |
|---|---|---|
| alpha-amino acids | R | R′ |
| L-Alanine (A) | —CH₃ | —H |
| L-Arginine (R) | —CH₂CH₂CH₂—NHC(═NH)NH₂ | —H |
| L-Asparagine (N) | —CH₂C(═O)NH₂ | —H |
| L-Aspartic acid (D) | —CH₂CO₂H | —H |
| L-Cysteine (C) | —CH₂SH | —H |
| L-Glutamic acid (E) | —CH₂CH₂CO₂H | —H |
| L-Glutamine (Q) | —CH₂C(═O)NH₂ | —H |
| Glycine (G) | —H | —H |
| L-Histidine (H) | —CH₂-2-(1H-imidazole) | —H |
| L-Isoleucine (I) | -sec-butyl | —H |
| L-Leucine (L) | -iso-butyl | —H |
| L-Lysine (K) | —CH₂CH₂CH₂CH₂NH₂ | —H |
| L-Methionine (M) | —CH₂CH₂SCH₃ | —H |
| L-Phenylalanine (F) | —CH₂Ph | —H |
| L-Proline (P) | -2-(pyrrolidine) | —H |
| L-Serine (S) | —CH₂OH | —H |
| L-Threonine (T) | —CH₂CH(OH)(CH₃) | —H |
| L-Tryptophan (W) | —CH₂-3-(1H-indole) | —H |
| L-Tyrosine (Y) | —CH₂-(p-hydroxyphenyl) | —H |
| L-Valine (V) | -isopropyl | —H |

TABLE 2

| | Amino acid side chains | |
|---|---|---|
| Exemplary unnatural alpha-amino acids | R | R′ |
| D-Alanine | —H | —CH₃ |
| D-Arginine | —H | —CH₂CH₂CH₂—NHC(═NH)NH₂ |
| D-Asparagine | —H | —CH₂C(═O)NH₂ |
| D-Aspartic acid | —H | —CH₂CO₂H |
| D-Cysteine | —H | —CH₂SH |
| D-Glutamic acid | —H | —CH₂CH₂CO₂H |
| D-Glutamine | —H | —CH₂CH₂C(═O)NH₂ |
| D-Histidine | —H | —CH₂-2-(1H-imidazole) |
| D-Isoleucine | —H | -sec-butyl |
| D-Leucine | —H | -iso-butyl |
| D-Lysine | —H | —CH₂CH₂CH₂CH₂NH₂ |
| D-Methionine | —H | —CH₂CH₂SCH₃ |
| D-Phenylalanine | —H | —CH₂Ph |
| D-Proline | —H | -2-(pyrrolidine) |
| D-Serine | —H | —CH₂OH |
| D-Threonine | —H | —CH₂CH(OH)(CH₃) |
| D-Tryptophan | —H | —CH₂-3-(1H-indole) |
| D-Tyrosine | —H | —CH₂-(p-hydroxyphenyl) |
| D-Valine | —H | -isopropyl |
| Di-vinyl | —CH═CH₂ | —CH═CH₂ |
| Exemplary unnatural alpha-amino acids | R and R′ are equal to: | |
| α-methyl-Alanine (Aib) | —CH₃ | —CH₃ |
| α-methyl-Arginine | —CH₃ | —CH₂CH₂CH₂—NHC(═NH)NH₂ |
| α-methyl-Asparagine | —CH₃ | —CH₂C(═O)NH₂ |
| α-methyl-Aspartic acid | —CH₃ | —CH₂CO₂H |
| α-methyl-Cysteine | —CH₃ | —CH₂SH |
| α-methyl-Glutamic acid | —CH₃ | —CH₂CH₂CO₂H |
| α-methyl-Glutamine | —CH₃ | —CH₂CH₂C(═O)NH₂ |
| α-methyl-Histidine | —CH₃ | —CH₂-2-(1H-imidazole) |
| α-methyl-Isoleucine | —CH₃ | -sec-butyl |
| α-methyl-Leucine | —CH₃ | -iso-butyl |
| α-methyl-Lysine | —CH₃ | —CH₂CH₂CH₂CH₂NH₂ |
| α-methyl-Methionine | —CH₃ | —CH₂CH₂SCH₃ |
| α-methyl-Phenylalanine | —CH₃ | —CH₂Ph |
| α-methyl-Proline | —CH₃ | -2-(pyrrolidine) |
| α-methyl-Serine | —CH₃ | —CH₂OH |
| α-methyl-Threonine | —CH₃ | —CH₂CH(OH)(CH₃) |
| α-methyl-Tryptophan | —CH₃ | —CH₂-3-(1H-indole) |
| α-methyl-Tyrosine | —CH₃ | —CH₂-(p-hydroxyphenyl) |
| α-methyl-Valine | —CH₃ | -isopropyl |
| Di-vinyl | —CH=CH₂ | —CH=CH₂ |
| Norleucine | —H | —CH₂CH₂CH₂CH₃ |

TABLE 3
Amino acid side chains

| Exemplary unnatural alpha-amino | |
|---|---|
| acids | R and R′ is equal to hydrogen or —CH₃, and: |
| Terminally unsaturated alpha-amino | —(CH₂)_{g}—S—(CH₂)_{g}CH═CH₂, |
| acids and bis alpha-amino acids (e.g., | —(CH₂)_{g}—O—(CH₂)_{g}CH═CH₂, |
| modified cysteine, modified lysine, | —(CH₂)_{g}—NH—(CH₂)_{g}CH═CH₂, |
| modified tryptophan, modified serine, | —(CH₂)_{g}—(C═O)—S—(CH₂)_{g}CH═CH₂, |
| modified threonine, modified proline, | —(CH₂)_{g}—(C═O)—O—(CH₂)_{g}CH═CH₂, |
| modified histidine, modified alanine, | —(CH₂)_{g}—(C═O)—NH—(CH₂)_{g}CH═CH₂, |
| and the like). | —CH₂CH₂CH₂CH₂—NH—(CH₂)_{g}CH═CH₂, |
| | —(C₆H₅)—p—O—(CH₂)_{g}CH═CH₂, |
| | —CH(CH₃)—O—(CH₂)_{g}CH═CH₂, |
| | —CH₂CH(—O—CH═CH₂)(CH₃), |
| | —histidine—N((CH₂)_{g}CH═CH₂), |
| | —tryptophan—N((CH₂)_{g}CH═CH₂), and |
| | —(CH₂)_{g+1}(CH═CH₂), |
| | wherein: |
| | each instance of g is, independently, 0 to 10. |

Exemplary unnatural alpha-amino acids

| | |
|---|---|
| 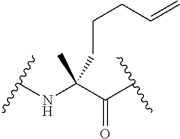 [SMILES: *N[C@](C)(CCCC=C)C(*)=O] | R5 |
| 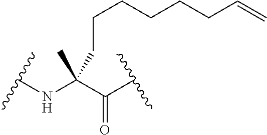 [SMILES: *N[C@](C)(CCCCCCC=C)C(*)=O] | R8 |
| 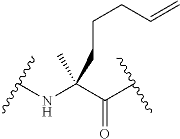 [SMILES: *N[C@@](C)(CCCC=C)C(*)=O] | S5 |
| 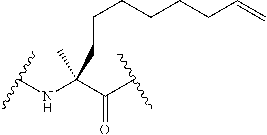 [SMILES: *N[C@@](C)(CCCCCCC=C)C(*)=O] | S8 |
| 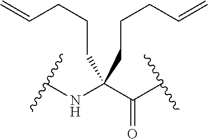 | B5 |
| 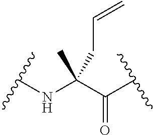 [SMILES: *N[C@](C)(CC=C)C(*)=O] | R3 |
| 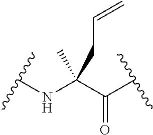 [SMILES: *N[C@@](C)(CC=C)C(*)=O] | S3 |
| 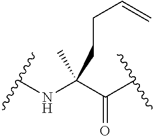 [SMILES: *N[C@@](C)(CCC=C)C(*)=O] | S4 |
| 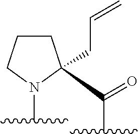 [SMILES: *C(=O)[C@]1(CC=C)CCCN1*] | PR3 |
| 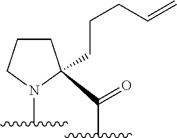 [SMILES: *C(=O)[C@]1(CCCC=C)CCCN1*] | RS5 |
| 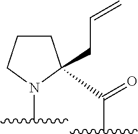 [SMILES: *C(=O)[C@@]1(CC=C)CCCN1*] | PS3 |
| 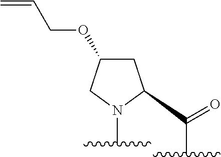 [SMILES: *C(=O)[C@@H]1C[C@@H](OCC=C)CN1*] | PSO3 |

There are many known unnatural amino acids any of which may be included in the peptides of the present invention. See for example, S. Hunt, The Non-Protein Amino Acids: In Chemistry and Biochemistry of the Amino Acids, edited by G. C. Barrett. Chapman and Hall, 1985. Some examples of unnatural amino acids are 4-hydroxyproline, desmosine, gamma-aminobutyric acid, beta-cyanoalanine, norvaline, 4-(E)-butenyl-4(R)-methyl-N-methyl-L-threonine, N-methyl-L-leucine, 1-amino-cyclopropanecarboxylic acid, 1-amino-2-phenyl-cyclopropanecarboxylic acid, 1-amino-cyclobutanecarboxylic acid, 4-amino-cyclopentenecarboxylic acid, 3-amino-cyclohexanecarboxylic acid, 4-piperidylacetic acid, 4-amino-1-methylpyrrole-2-carboxylic acid, 2,4-diaminobutyric acid, 2,3-diaminopropionic acid, 2,4-diaminobutyric acid, 2-aminoheptanedioic acid, 4-(aminomethyl)benzoic acid, 4-aminobenzoic acid, ortho-, meta- and para-substituted phenylalanines (e.g., substituted with —C(═O)C₆H₅; —CF₃; —CN; -halo; —NO₂; CH₃), disubstituted phenylalanines, substituted tyrosines (e.g., further substituted with —C(═O)C₆H₅; —CF₃; —CN; -halo; —NO₂; CH₃), and statine. Additionally, the amino acids suitable for use in the present invention may be derivatized to include amino acid residues that are hydroxylated, phosphorylated, sulfonated, acylated, and glycosylated, to name a few.

The term “amino acid side chain” refers to a group attached to the alpha- or beta-carbon of an amino acid. A “suitable amino acid side chain” includes, but is not limited to, any of the suitable amino acid side chains as defined above, and as provided in Tables 1 to 3.

For example, suitable amino acid side chains include methyl (as the alpha-amino acid side chain for alanine is methyl), 4-hydroxyphenylmethyl (as the alpha-amino acid side chain for tyrosine is 4-hydroxyphenylmethyl) and thiomethyl (as the alpha-amino acid side chain for cysteine is thiomethyl), etc. A “terminally unsaturated amino acid side chain” refers to an amino acid side chain bearing a terminal unsaturated moiety, such as a substituted or unsubstituted, double bond (e.g., olefinic) or a triple bond (e.g., acetylenic), that participates in crosslinking reaction with other terminal unsaturated moieties in the polypeptide chain. In certain embodiments, a “terminally unsaturated amino acid side chain” is a terminal olefinic amino acid side chain. In certain embodiments, a “terminally unsaturated amino acid side chain” is a terminal acetylenic amino acid side chain. In certain embodiments, the terminal moiety of a “terminally unsaturated amino acid side chain” is not further substituted. Terminally unsaturated amino acid side chains include, but are not limited to, side chains as depicted in Table 3.

A “peptide” or “polypeptide” comprises a polymer of amino acid residues linked together by peptide (amide) bonds. The term(s), as used herein, refers to proteins, polypeptides, and peptide of any size, structure, or function. Typically, a peptide or polypeptide will be at least three amino acids long. A peptide or polypeptide may refer to an individual protein or a collection of proteins. Inventive proteins preferably contain only natural amino acids, although non-natural amino acids (i.e., compounds that do not occur in nature but that can be incorporated into a polypeptide chain) and/or amino acid analogs as are known in the art may alternatively be employed. One or more of the amino acids in a peptide or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification. A peptide or polypeptide may also be a single molecule or may be a multi-molecular complex, such as a protein. A peptide or polypeptide may be just a fragment of a naturally occurring protein or peptide. A peptide or polypeptide may be naturally occurring, recombinant, or synthetic, or any combination thereof.

As used herein “dipeptide” refers to two covalently linked amino acids.

As used herein, the term “salt” or “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, quaternary salts, e.g., cationic trisubstituted amino groups, e.g., as defined herein.

The following definitions are more general terms used throughout the present application.

The term “subject,” as used herein, refers to any animal. In certain embodiments, the subject is a mammal. In certain embodiments, the term “subject”, as used herein, refers to a human (e.g., a man, a woman, or a child).

The terms “administer,” “administering,” or “administration,” as used herein refers to implanting, absorbing, ingesting, injecting, or inhaling, the inventive polypeptide or compound.

The terms “treat” or “treating,” as used herein, refers to partially or completely alleviating, inhibiting, ameliorating, and/or relieving the disease or condition from which the subject is suffering.

The terms “effective amount” and “therapeutically effective amount.” as used herein, refer to the amount or concentration of a biologically active agent conjugated to a stitched or stapled polypeptide as described herein, or amount or concentration of a stitched or stapled polypeptide as described herein, that, when administered to a subject, is effective to at least partially treat a condition from which the subject is suffering.

As used herein, when two entities are “conjugated” to one another they are linked by a direct or indirect covalent or non-covalent interaction. In certain embodiments, the association is covalent. In other embodiments, the association is non-covalent. Non-covalent interactions include hydrogen bonding, van der Waals interactions, hydrophobic interactions, magnetic interactions, and electrostatic interactions. An indirect covalent interaction is when two entities are covalently connected, optionally through a linker group.

As used herein, a “biologically active agent” or “therapeutically active agent” refers to any substance used as a medicine for treatment, prevention, delay, reduction or amelioration of a disease, condition, or disorder, and refers to a substance that is useful for therapy, including prophylactic and therapeutic treatment. A biologically active agent also includes a compound that increases the effect or effectiveness of another compound, for example, by enhancing potency or reducing adverse effects of the other compound.

In certain embodiments, a biologically active agent is an anti-cancer agent, antibiotic, anti-viral agent, anti-HIV agent, anti-parasite agent, anti-protozoal agent, anesthetic, anticoagulant, inhibitor of an enzyme, steroidal agent, steroidal or non-steroidal anti-inflammatory agent, antihistamine, immunosuppressant agent, anti-neoplastic agent, antigen, vaccine, antibody, decongestant, sedative, opioid, analgesic, anti-pyretic, birth control agent, hormone, prostaglandin, progestational agent, anti-glaucoma agent, ophthalmic agent, anti-cholinergic, analgesic, anti-depressant, anti-psychotic, neurotoxin, hypnotic, tranquilizer, anti-convulsant, muscle relaxant, anti-Parkinson agent, anti-spasmodic, muscle contractant, channel blocker, miotic agent, anti-secretory agent, anti-thrombotic agent, anticoagulant, anti-cholinergic, β-adrenergic blocking agent, diuretic, cardiovascular active agent, vasoactive agent, vasodilating agent, anti-hypertensive agent, angiogenic agent, modulators of cell-extracellular matrix interactions (e.g. cell growth inhibitors and anti-adhesion molecules), or inhibitors/intercalators of DNA, RNA, protein-protein interactions, protein-receptor interactions, etc.

Exemplary biologically active agents include, but are not limited to, small organic molecules such as drug compounds, peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the biologically active agent is a cell. Exemplary cells include immune system cells (e.g., mast, lymphocyte, plasma cell, macrophage, dendritic cell, neutrophils, eosinophils), connective tissue cells (e.g., blood cells, erythrocytes, leucocytes, megakaryocytes, fibroblasts, osteoclasts), stem cells (e.g., embryonic stem cells, adult stem cells), bone cells, glial cells, pancreatic cells, kidney cells, nerve cells, skin cells, liver cells, muscle cells, adipocytes, Schwann cells, Langerhans cells, as well as (micro)-tissues such as the Islets of Langerhans.

In certain embodiments, the biologically active agent is a small organic molecule. In certain embodiments, a small organic molecule is non-peptidic. In certain embodiments, a small organic molecule is non-oligomeric. In certain embodiments, a small organic molecule is a natural product or a natural product-like compound having a partial structure (e.g., a substructure) based on the full structure of a natural product. Exemplary natural products include steroids, penicillins, prostaglandins, venoms, toxins, morphine, paclitaxel (Taxol), morphine, cocaine, digitalis, quinine, tubocurarine, nicotine, muscarine, artemisinin, cephalosporins, tetracyclines, aminoglycosides, rifamycins, chloramphenicol, asperlicin, lovastatin, ciclosporin, curacin A, eleutherobin, discodermolide, bryostatins, dolostatins, cephalostatins, antibiotic peptides, epibatidine, α-bungarotoxin, tetrodotoxin, teprotide, and neurotoxins from Clostridium botulinum. In certain embodiments, a small organic molecule is a drug approved by the Food and Drugs Administration as provided in the Code of Federal Regulations (CFR).

As used herein, a “label” refers to a moiety that has at least one element, isotope, or functional group incorporated into the moiety which enables detection of the inventive polypeptide to which the label is attached. Labels can be directly attached (ie, via a bond) or can be attached by a linker (e.g. such as, for example, a cyclic or acyclic, branched or unbranched, substituted or unsubstituted alkylene; cyclic or acyclic, branched or unbranched, substituted or unsubstituted alkenylene; cyclic or acyclic, branched or unbranched, substituted or unsubstituted alkynylene; cyclic or acyclic, branched or unbranched, substituted or unsubstituted heteroalkylene; cyclic or acyclic, branched or unbranched, substituted or unsubstituted heteroalkenylene; cyclic or acyclic, branched or unbranched, substituted or unsubstituted heteroalkynylene; substituted or unsubstituted arylene; substituted or unsubstituted heteroarylene; or substituted or unsubstituted acylene, or any combination thereof, which can make up a linker). It will be appreciated that the label may be attached to the inventive polypeptide at any position that does not interfere with the biological activity or characteristic of the inventive polypeptide that is being detected.

In general, a label can fall into any one (or more) of five classes: a) a label which contains isotopic moieties, which may be radioactive or heavy isotopes, including, but not limited to, ²H, ³H, ¹³C, ¹⁴C, ¹⁴N, ³¹P, ³²P, ³⁵S, ⁶⁷Ga, ^{99m}Tc (Tc-99m), ¹¹¹In, ¹²³I, ¹²⁵I, ¹⁶⁹Yb, and ¹⁸⁶Re; b) a label which contains an immune moiety, which may be antibodies or antigens, which may be bound to enzymes (e.g., such as horseradish peroxidase); c) a label which is a colored, luminescent, phosphorescent, or fluorescent moieties (e.g., such as the fluorescent label FITC); d) a label which has one or more photoaffinity moieties; and e) a label which has a ligand moiety with one or more known binding partners (such as biotin-streptavidin, FK506-FKBP, etc.). Any of these type of labels as described above may also be referred to as “diagnostic agents” as defined herein.

In certain embodiments, such as in the identification of a biological target, label comprises a radioactive isotope, preferably an isotope which emits detectable particles, such as p particles. In certain embodiments, the label comprises one or more photoaffinity moieties for the direct elucidation of intermolecular interactions in biological systems. A variety of known photophores can be employed, most relying on photoconversion of diazo compounds, azides, or diazirines to nitrenes or carbenes (see, Bayley, H., Photogenerated Reagents in Biochemistry and Molecular Biology (1983), Elsevier, Amsterdam, the entire contents of which are incorporated herein by reference). In certain embodiments of the invention, the photoaffinity labels employed are o-, m- and p-azidobenzoyls, substituted with one or more halogen moieties, including, but not limited to 4-azido-2,3,5,6-tetrafluorobenzoic acid.

In certain embodiments, the label comprises one or more fluorescent moieties. In certain embodiments, the label is the fluorescent label FITC. In certain embodiments, the label comprises a ligand moiety with one or more known binding partners. In certain embodiments, the label comprises the ligand moiety biotin.

As used herein, a “diagnostic agent” refers to imaging agents. Exemplary imaging agents include, but are not limited to, those used in positron emissions tomography (PET), computer assisted tomography (CAT), single photon emission computerized tomography, x-ray, fluoroscopy, and magnetic resonance imaging (MRI); anti-emetics; and contrast agents. Exemplary diagnostic agents include but are not limited to, fluorescent moieties, luminescent moieties, magnetic moieties; gadolinium chelates (e.g., gadolinium chelates with DTPA, DTPA-BMA, DOTA and HP-DO3A), iron chelates, magnesium chelates, manganese chelates, copper chelates, chromium chelates, iodine-based materials useful for CAT and x-ray imaging, and radionuclides. Suitable radionuclides include, but are not limited to, ¹²³I, ¹²⁵I, ¹³⁰I, ¹³¹I, ¹³³I, ¹³⁵I, ⁴⁷Sc, ⁷²As, ⁷²Se, ⁹⁰Y, ⁸⁸Y, ⁹⁷Ru, ¹⁰⁰Pd, ¹⁰¹mRh, ¹¹⁹Sb, ¹²⁸Ba, ¹⁹⁷Hg, ²¹¹At, ²¹²Bi, ²¹²Pb, ¹⁰⁹Pd, ¹¹¹In, ⁶⁷Ga, ⁶⁸Ga, ⁶⁷Cu, ⁷⁵Br, ⁷⁷Br, ⁹⁹mTc, ¹⁴C, ¹³N, ¹⁵O, ³²P, ³³P, and ¹⁸F. Fluorescent and luminescent moieties include, but are not limited to, a variety of different organic or inorganic small molecules commonly referred to as “dyes,” “labels,” or “indicators.” Examples include, but are not limited to, fluorescein, rhodamine, acridine dyes, Alexa dyes, cyanine dyes, etc. Fluorescent and luminescent moieties may include a variety of naturally occurring proteins and derivatives thereof, e.g., genetically engineered variants. For example, fluorescent proteins include green fluorescent protein (GFP), enhanced GFP, red, blue, yellow, cyan, and sapphire fluorescent proteins, reef coral fluorescent protein, etc. Luminescent proteins include luciferase, aequorin and derivatives thereof. Numerous fluorescent and luminescent dyes and proteins are known in the art (see, e.g., U.S. Patent Publication 2004/0067503; Valeur, B., “Molecular Fluorescence: Principles and Applications,” John Wiley and Sons, 2002; and Handbook of Fluorescent Probes and Research Products, Molecular Probes, 9^{th} edition, 2002).

### DETAILED DESCRIPTION OF CERTAIN EMBODIMENTS OF THE INVENTION

In one aspect, the disclosure provides stabilized stapled peptides with a proline derivative for stapling at the N-terminus of the helix and methods for increasing the stability of peptides using a proline-derivative for stapling. In some embodiments, the disclosure provides peptides with increased alpha-helicity and methods for increasing the alpha-helicity of peptides. In some embodiments, the disclosure provides stapled peptides with a proline derivative at the N-terminus of the alpha-helix and methods for providing such stapled peptides. In some embodiments, the disclosure provides proline-locked stapled peptides and methods for providing proline-locked stapled peptides.

In one aspect, the disclosure provides peptides that are stabilized by stapling the peptide at the N-terminus of an alpha-helix through the introduction of a proline-containing staple or a proline-locked staple. It was surprisingly found that proline could be used to stabilize peptides. The finding was surprising at least because proline is commonly considered an α-helix-disrupting amino acid. In some embodiments, the proline-locked stapled peptide includes a proline at position i that is covalently connected with the alpha-carbon of a second amino acid at position i+3. While alpha-helical peptides are relatively stable once formed, initiation of alpha helix formation is challenging because the attendant conformational ordering is entropically expensive (J. Chem. Phys, 1959, 31, 526-535). As provided herein, introducing a helix staple, such as a proline staple or a proline-locked staple at the N-terminus of an alpha helical peptide helps with the formation of, and further stabilizes, an alpha-helix. Once a single turn of the α-helix is formed, its downstream propagation can occur spontaneously, provided that helix-disruption sequences are not present.

In one aspect, the disclosure provides a peptide stapling system having helix-nucleating ability. In some embodiments, the peptide stapling system is a peptide with a proline-derivative at the N-terminus of the staple. In some embodiments, the peptide stapling system is a proline-locked stapled peptide or “Pro-lock”. It should be appreciated that the peptide stabilized by a proline-lock may be a peptide that is mostly in alpha-helical conformation, or the peptide may be part of a larger protein that includes one or more alpha-helical regions. In some embodiments, the Pro-locked staple is located in the N-terminal region of a peptide. In some embodiments, the proline of the Pro-locked staple is located at the N-terminal position of the helix. In some embodiments, the Pro-locked staple comprises a covalent bind between a proline at position i and a second amino acid at position i+3 in a peptide. It should be appreciated that homo-proline and other unnatural cyclic amino acids, as described further herein, can be used instead of proline in the proline locks. To facilitate to covalent bond of the proline-lock, the proline comprises an additional functional group that can undergo a reaction to for a covalent bond. In some embodiments, the functional group is a double bond (e.g., a vinyl group). In some embodiments, the functional group is located at the alpha-carbon on the proline. In some embodiments, the functional group is located on any position on the proline ring.

In some embodiments, the amino acid at position i+3 is serine, alanine, glycine, aspartic acid or glutamic acid. To facilitate the formation of the covalent bond of the proline-lock the amino acid at position i+3 may include an additional functional group that can undergo a reaction to form a covalent bond. In some embodiments, the additional functional group that can undergo a reaction to form a covalent bond is located at the alpha carbon of the second amino acid. In some embodiments, the group that can undergo a reaction to form a covalent bond is part of the natural side chain of the amino acid.

In some embodiments, the proline locked staple includes a covalent binding between a proline at position i and an amino acid located at position i+3. In some embodiments, the helix-nucleating “staple” is formed between an N-terminal α-allylproline (e.g., P_{R3}) and an α-methyl,α-allylglycine (S₃) at positions i and i+3 in a peptide. In some embodiments, the helix-nucleating “staple” is formed between (R)—N-(Acetyl)-2-(2′-propenyl)proline (“P_{R3}”) or (R)—N-[(9H-Fluoren-9-ylmethoxy)carbonyl]-2-(2′-propenyl)proline and (S)—N-[(9H-Fluoren-9-ylmethoxy)carbonyl]-2-(2′-propylenyl)alanine at positions i and i+3 in a peptide.

The unnatural amino acids of the proline-lock can be introduced into the peptide through peptide synthesis techniques as described herein. In some embodiments, the amino acid sequence including the proline lock is synthesized or prepared separately and the amino acid sequence is coupled to a peptide to be stabilized. Thus, in some embodiments, the disclosure provides a method of increasing the stability and/or helicity of peptide that include a step of coupling the peptide to an amino acid sequence comprising a proline-locked staple.

The proline-locked peptides comprising the covalent bond may be synthesized according to any of the methods disclosed herein. In some embodiments, a crosslink between the proline with the functional group and the amino acid at position i+3 is formed by Grubb's catalyst. In some embodiments, a crosslink between the proline with the functional group and the amino acid at position i+3 is formed by ruthenium-mediated olefin metathesis. In some embodiment, the Pro-locked stapled peptides are synthesized using (R)—N-(Acetyl)-2-(2′-propenyl)proline and (S)—N-[(9H-Fluoren-9-ylmethoxy)carbonyl]-2-(2′-propylenyl)alanine as amino acid building blocks at position i and i+3, respectively, allowing for the generation of a proline-locked stapled peptide. In some embodiments, the peptides are subjected to ruthenium-mediated olefin metathesis, resulting in formation of an exclusively cis olefinic crosslink.

In some embodiments, the peptides provided herein comprise stabilizing elements in addition to the proline-locked staple. In some embodiments, the peptides comprise multiple pro-locked staples. In some embodiments, the peptides comprise a Pro-locked staples and a staple other than a Pro-locked staple. Peptide staples other than Pro-locked staples are provided for instance in WO2008/121767. In general, it has been shown that the pharmacologic properties of α-helical peptides can be greatly improved through the use of a hydrocarbon “staple” that enforces the α-helical conformation of peptides (See e.g., Science, 2004, 305, 1466-1470). In some embodiments, the proline-locked staple and a second staple connect at amino acid i+3 or overlap in amino acid sequence. Thus, for instance, in addition to a proline-locked amino staple between i and i+3, a peptide may have a second staple that starts at position i+3 (e.g., between i+3 and i+7), or a second staple that starts at position i+1 or i+2, and thus “overlaps” with the proline-locked staple. Compared to stapled peptides disclosed previously, the Pro-locked stapled peptides disclosed herein have the extra advantage that they can be used even when a crosslink cannot be introduced into any position of an α-helix other than at its N-terminus.

In some embodiments, the peptides comprising the proline-locked staples may have additional stabilizing elements. In some embodiments, the peptides have an amino acid composition allowing for helix stabilizing salt bridges. In some embodiments, the peptides have been modified to covalently connect the salt bridges. In some embodiments, the peptides have functional groups that stabilize the helix dipole.

In one aspect, the disclosure provides peptides with an improved ability to cross cell membranes. An increased ability of peptides to cross the cell membrane is correlated with an increase in the capacity of the peptide to acts as a therapeutic. Peptides often have difficulty crossing (cell) membranes because of the availability of unpaired hydrogen bonds in the peptide (e.g., in the peptide backbone). The disclosure provides methods for minimizing the availability of unpaired hydrogen bonds in a peptide by binding N-terminal amide protons tightly into hydrogen-bonding interactions. As disclosed herein, locating an amino acid with a side chain that can interact with amide protons at the N-terminal side of an alpha helix minimizes the availability of unwanted amide protons. The undesired free N-terminal amide proteins are “masked” thereby minimizing any undesired interactions with other agents (e.g., the cell membrane or components thereof). In some embodiments, the amino acid with the side chain that can interact with amide protons is modified to increase the available hydrogen binders. For instance, the disclosure provides a modified arginine with increased ability to mask N-terminal amide protons by providing additional hydrogen-bonding interaction partners.

In one aspect, the disclosure provides methods and compositions for improving pharmacological properties of peptides. In some embodiments, the disclosure provides peptides with improved capacity for passive cell penetration (e.g., by improved capacity for passive cell membrane traversal). In some embodiments, the disclosure provides methods for improving the passive cell penetration of peptides. In some embodiments, the disclosure provides peptides with minimized unwanted N-terminal amide N—H proton interactions. In some embodiments, the disclosure provides methods for generating peptides with minimized unwanted amide N—H proton interactions. Decreasing the availability of freely available hydrogen bonds in N—H protons will minimize the interactions the peptide will have with third parties (e.g. a membrane or membrane components) allowing for better traversal of the membrane. In some embodiments, the disclosure provides peptides with improved passive cell penetration and minimized amide N—H proton interactions. In some embodiments, the disclosure provides methods for improving the passive cell penetration of peptides by minimizing amide N—H proton interactions. In some embodiments, the peptides with improved passive cell penetration are proline-locked staple peptides. In some embodiments, the peptides with improved passive cell penetration have minimized amide N—H bond interactions by “cloaking” or “masking” the amide N—H's. In some embodiments, the peptides with minimized amide N—H interactions have minimized the interactions of amide N—H's located at the N-terminus of the peptide. In some embodiments, the peptides with improved passive cell penetration are proline-locked staple peptides with minimized amide N—H bond interactions. In some embodiments, the amide N—H interactions are minimized by introducing an amino acid with a negatively charged side chain and/or electron donor on its side chain on the N-terminal side of the polypeptide. In some embodiments, the amide N—H interactions are minimized by introducing an amino acid with a negatively charged side chain and/or electron donor on its side chain on the N-terminal side of a helix within the polypeptide. In some embodiments, the amino acid allowing for the minimization of N—H proton interactions is serine, threonine, aspartic acid, glutamic acid or asparagine. In some embodiments, the amino acid is has been modified to increase the number of electron donating groups on the side chain. In some embodiments, the amino acid is a modified asparagine as disclosed herein (also called “asparagine surrogate”).

In one aspect, the disclosure provides stabilized peptides that nucleate α-helix formation through a proline-locked staple while also binding N-terminal amide protons tightly through hydrogen-bonding interactions. As provided herein, the stabilized peptides with amide proton binding can have a proline at position i that is covalently coupled to an amino acid at position i+3, and a modified arginine at position i−1 which interacts with the amide protons of the peptide backbone of the amino acids at position i+1 and i+2

Promotion of α-helix stability and masking of N-terminal N—H's improve the biophysical and pharmacological properties of a peptide, including oral bioavailability, binding affinity for a receptor, resistance to proteolytic degradation, cell-penetration, and reduction in the rate of renal clearance. The proline-locked stapled peptides provided herein are strong nucleators of α-helix formation, as shown by the exceptionally high helicity of peptides bearing the proline-lock. In addition, the peptides provided herein, through masking the N-terminal amide protons, further enhance the ability of the peptides to cross cell membranes. Thus, the Pro-locked stapled peptides provided herein can be used in targeting previously “undruggable” intracellular therapeutic targets.

### Polypeptides and Precursors

Various stapled and stitched polypeptides are described herein which include proline-locked staple. “Peptide stapling” is a term coined from a synthetic methodology wherein two olefin-containing sidechains present in a polypeptide chain are covalently joined (e.g., “stapled together”) using a ring-closing metathesis (RCM) reaction to form a cross-linked ring (see, the cover art for J. Org. Chem. (2001) vol. 66, issue 16 describing metathesis-based crosslinking of alpha-helical peptides; Blackwell et al.; Angew Chem. Int. Ed (1994) 37:3281). However, the term “peptide stapling,” as used herein, encompasses the joining of two double bond-containing sidechains, two triple bond-containing sidechains, or one double bond-containing and one triple bond-containing side chain, which may be present in a polypeptide chain, using any number of reaction conditions and/or catalysts to facilitate such a reaction, to provide a singly “stapled” polypeptide. Additionally, the term “peptide stitching,” as used herein, refers to multiple and tandem “stapling” events in a single polypeptide chain to provide a “stitched” (or multiply stapled) polypeptide.

The stapling or stitching contemplated herein involves contact of a precursor “unstapled” or “unstitched” polypeptide with a ring closing metathesis (RCM) catalyst to provide a stapled or stitched polypeptide. One of ordinary skill in the art will realize that a variety of RCM catalysts can be utilized. In certain embodiments, the RCM catalyst is a tungsten (W), molybdenum (Mo), or ruthenium (Ru) catalyst. In certain embodiments, the RCM catalyst is a ruthenium catalyst. Exemplary RCM catalysts employable by the above synthetic method may be described in Grubbs et al., Acc. Chem. Res. 1995, 28, 446-452; U.S. Pat. No. 5,811,515; Schrock et al., Organometallics (1982) 1 1645; Gallivan et al., Tetrahedron Letters (2005) 46:2577-2580; Furstner et al., J. Am. Chem. Soc. (1999) 121:9453; and Chem. Eur. J. (2001) 7:5299; the entire contents of each of which are incorporated herein by reference.

Thus, in one aspect, provided is a precursor polypeptide of Formula (P-I):

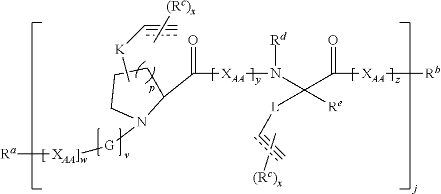

or a salt or stereoisomer thereof;
wherein:
each instance of K and L, is, independently, a bond or a group consisting of one or more combinations of substituted or unsubstituted alkylene; substituted or unsubstituted alkenylene; substituted or unsubstituted alkynylene; substituted or unsubstituted heteroalkylene; substituted or unsubstituted heteroalkenylene; substituted or unsubstituted heteroalkynylene; substituted or unsubstituted heterocyclene, substituted or unsubstituted carbocyclene, substituted or unsubstituted arylene; substituted or unsubstituted heteroarylene;
R^{a} is hydrogen; substituted or unsubstituted aliphatic; substituted or unsubstituted heteroaliphatic; substituted or unsubstituted aryl; substituted or unsubstituted heteroaryl; substituted or unsubstituted acyl; a resin; an amino protecting group; a label optionally joined by a linker, wherein the linker is a group consisting of one or more combinations of substituted or unsubstituted alkylene; substituted or unsubstituted alkenylene; substituted or unsubstituted alkynylene; substituted or unsubstituted heteroalkylene; substituted or unsubstituted heteroalkenylene; substituted or unsubstituted heteroalkynylene; substituted or unsubstituted carbocyclene; substituted or unsubstituted heterocyclene; substituted or unsubstituted arylene; or substituted or unsubstituted heteroarylene;
R^{b} is, —R^{B}, —OR^{B}, —N(R^{B})₂, or —SR^{B}, wherein each instance of R^{B} is, independently, hydrogen, substituted or unsubstituted aliphatic; substituted or unsubstituted heteroaliphatic; substituted or unsubstituted aryl; substituted or unsubstituted heteroaryl; substituted or unsubstituted acyl; a resin; a suitable hydroxyl, amino or thiol protecting group; or two R^{B} groups together form a substituted or unsubstituted 5- to 6-membered heterocyclic or heteroaromatic ring;
each instance of R^{c}, is, independently, hydrogen; substituted or unsubstituted aliphatic; substituted or unsubstituted heteroaliphatic; substituted or unsubstituted aryl; substituted or unsubstituted heteroaryl; substituted or unsubstituted acyl; substituted or unsubstituted hydroxyl; substituted or unsubstituted thiol; substituted or unsubstituted amino; cyano; isocyano; halo; or nitro;
each instance of R^{d} is, independently, hydrogen; substituted or unsubstituted aliphatic; substituted or unsubstituted heteroaliphatic; substituted or unsubstituted aryl; substituted or unsubstituted heteroaryl; substituted or unsubstituted acyl; or an amino protecting group;
each instance of R^{e} is, independently, hydrogen; substituted or unsubstituted aliphatic; substituted or unsubstituted heteroaliphatic; substituted or unsubstituted aryl; substituted or unsubstituted heteroaryl; substituted or unsubstituted acyl; substituted or unsubstituted hydroxyl; substituted or unsubstituted thiol; substituted or unsubstituted amino; cyano; isocyano; halo; or nitro;
each instance of G is, independently, a natural or unnatural amino acid or a group of the formula:

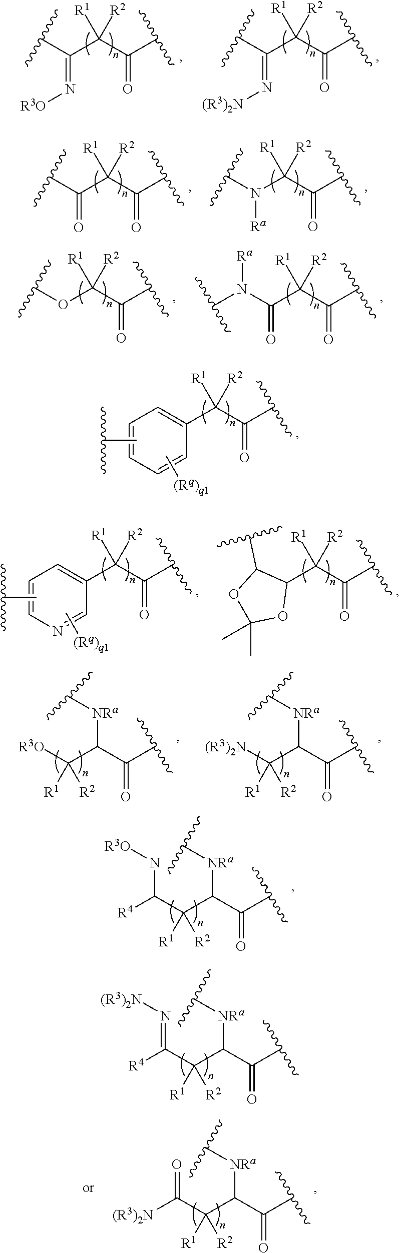

wherein:
n is 1, 2, or 3; and
each instance of R¹ and R² is independently hydrogen; substituted or unsubstituted aliphatic; substituted or unsubstituted heteroaliphatic; substituted or unsubstituted aryl; substituted or unsubstituted heteroaryl; substituted or unsubstituted acyl; substituted or unsubstituted hydroxyl; substituted or unsubstituted thiol; substituted or unsubstituted amino; or halo, or R¹ and R² are joined to form a carbocyclic or heterocyclic ring;
each instance of R³ and R⁴ is, independently, hydrogen; substituted or unsubstituted aliphatic; substituted or unsubstituted heteroaliphatic; a hydroxyl protecting group when attached to an oxygen atom, or an amino protecting group when attached to a nitrogen atom, or two R³ groups when attached to a nitrogen atom are joined to form a heterocyclic ring;
each instance of R^{q} is independently halogen, —CN, —NO₂, —N₃, optionally substituted alkyl, optionally substituted alkenyl, optionally substituted alkynyl, optionally substituted carbocyclyl, optionally substituted phenyl, optionally substituted heterocyclyl, optionally substituted heteroaryl, optionally substituted alkoxy, an optionally substituted amino group, or optionally substituted acyl;
q1 is 0, 1, 2, 3, or 4;
each instance of X_{AA} is, independently, a natural or unnatural amino acid;
j is, independently, an integer between 1 to 10, inclusive;
each instance of p is, independently, 1 or 2;
each instance of v is, independently, 0 or 1;
each instance of w and z is, independently, an integer between 0 and 100, inclusive;
each instance of x is, independently, 0, 1, 2, or 3;
y is, independently, an integer of 1 to 8, inclusive; and
[symbol] corresponds to a double or triple bond.

In certain embodiments, the [symbol] corresponds to a double bond. In certain embodiments, the [symbol] corresponds to a triple bond.

In certain embodiments, the polypeptide of Formula (P-I) is of the formula:

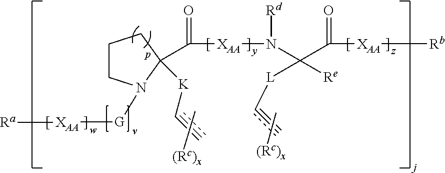

or a salt or stereoisomer thereof.

In certain embodiments, the polypeptide of Formula (P-I) is any one of the formula:

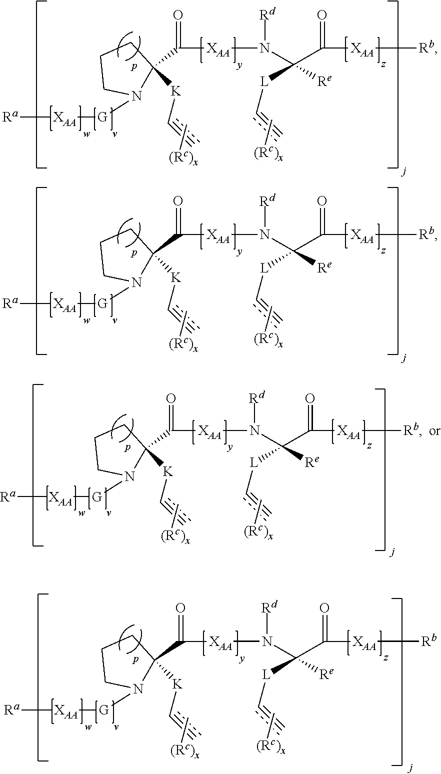

or a salt or stereoisomer thereof.

In certain embodiments, the polypeptide of Formula (P-I) is any one of the formula:

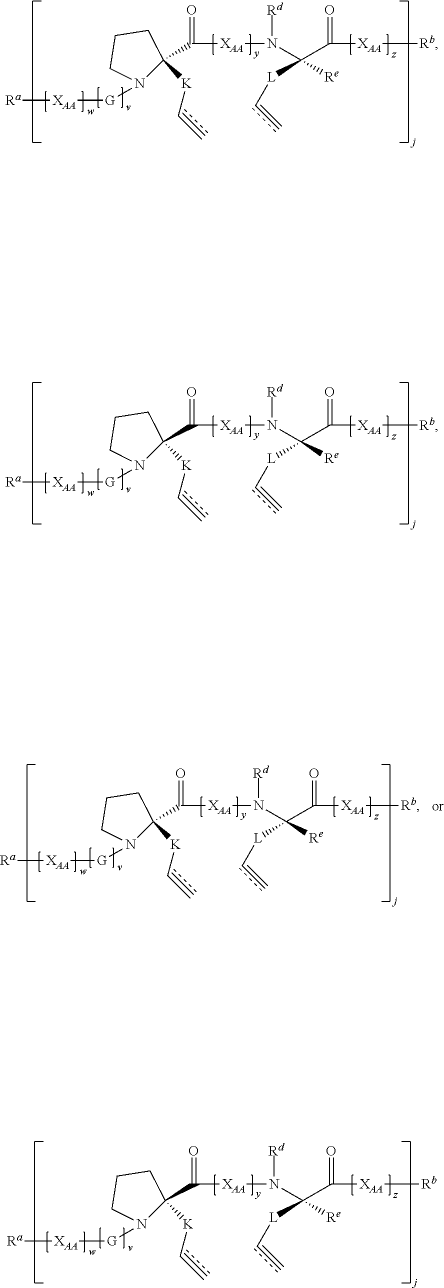

or a salt or stereoisomer thereof.

In certain embodiments, the polypeptide of Formula (P-I) is of the formula:

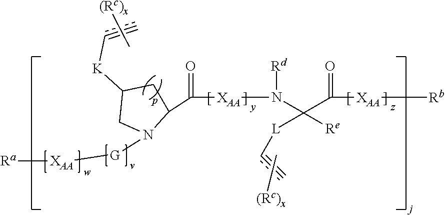

or a salt or stereoisomer thereof.

In certain embodiments, the polypeptide of Formula (P-I) is any one of the formulae:

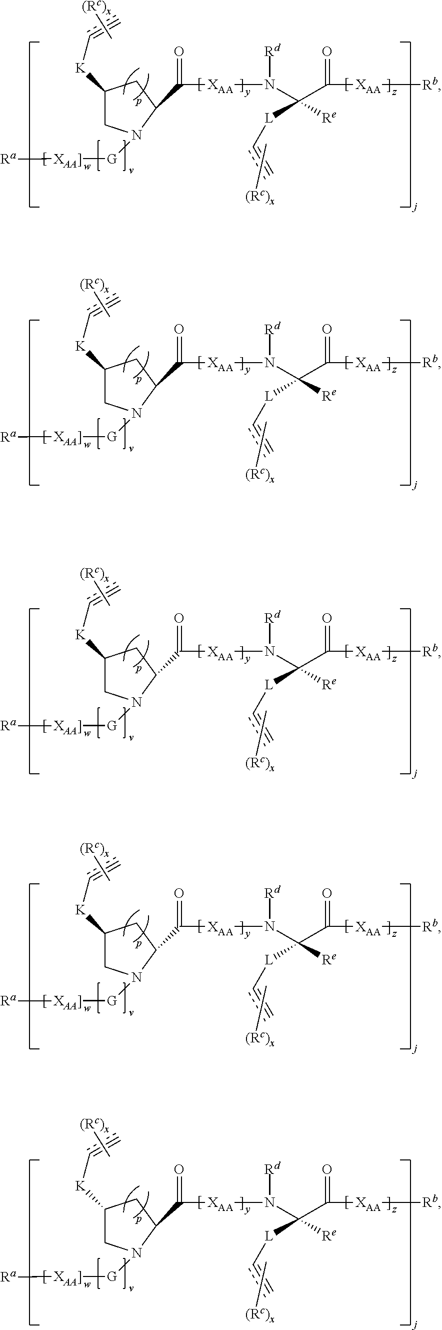

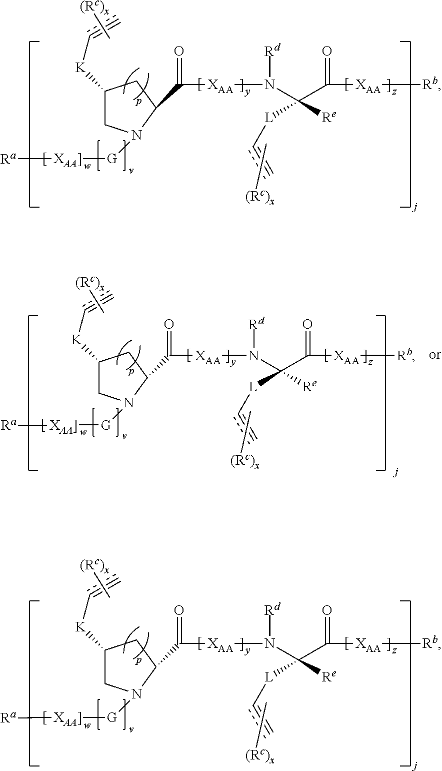

or a salt or stereoisomer thereof.

In certain embodiments, the polypeptide of Formula (P-I) is any one of the formulae:

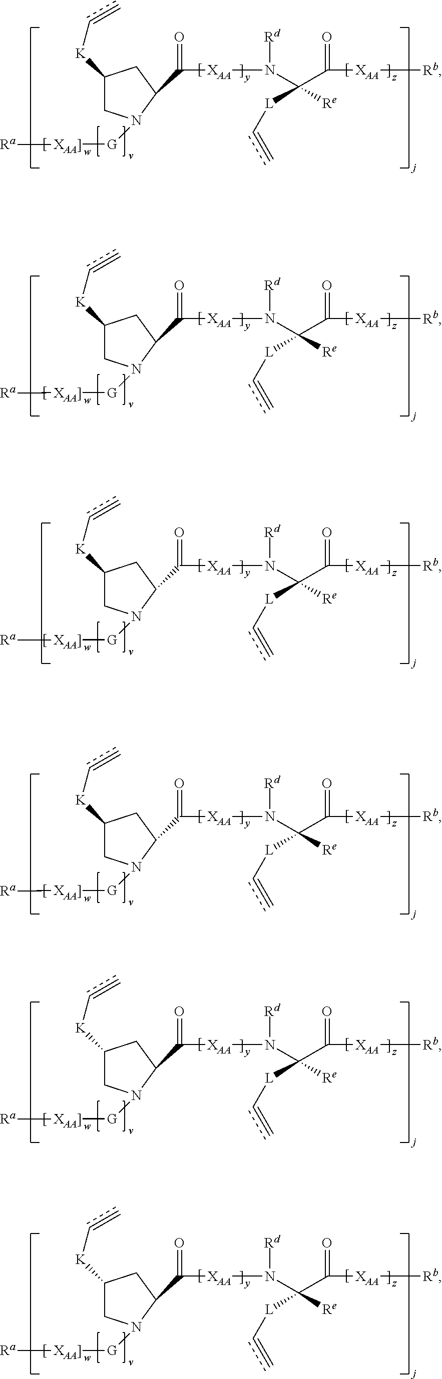

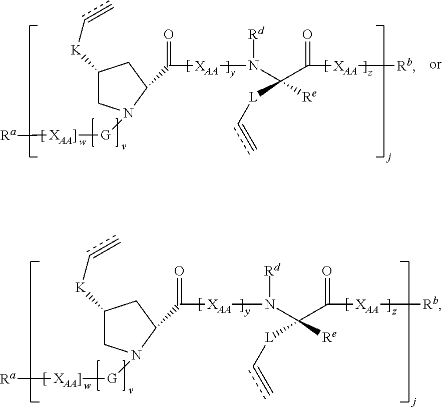

or a salt or stereoisomer thereof.

In certain embodiments, the precursor polypeptide of Formula (P-I), upon contact with a ring closing metathesis catalyst, generates a stapled polypeptide of Formula (I):

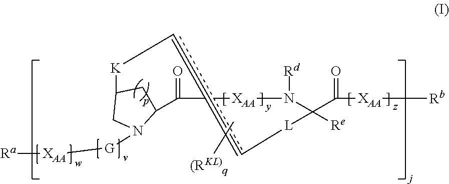

or a salt or stereoisomer thereof;
wherein:
each instance of K and L, is, independently, a bond or a group consisting of one or more combinations of substituted or unsubstituted alkylene; substituted or unsubstituted alkenylene; substituted or unsubstituted alkynylene; substituted or unsubstituted heteroalkylene; substituted or unsubstituted heteroalkenylene; substituted or unsubstituted heteroalkynylene; substituted or unsubstituted heterocyclene, substituted or unsubstituted carbocyclene, substituted or unsubstituted arylene; substituted or unsubstituted heteroarylene;
R^{a} is hydrogen; substituted or unsubstituted aliphatic; substituted or unsubstituted heteroaliphatic; substituted or unsubstituted aryl; substituted or unsubstituted heteroaryl; substituted or unsubstituted acyl; a resin; an amino protecting group; a label optionally joined by a linker, wherein the linker is a group consisting of one or more combinations of substituted or unsubstituted alkylene; substituted or unsubstituted alkenylene; substituted or unsubstituted alkynylene; substituted or unsubstituted heteroalkylene; substituted or unsubstituted heteroalkenylene; substituted or unsubstituted carbocyclene; substituted or unsubstituted heterocyclene; substituted or unsubstituted arylene; or substituted or unsubstituted heteroarylene;
R^{b} is, —R^{B}, —OR^{B}, —N(R^{B})₂, or —SR^{B}, wherein each instance of R^{B} is, independently, hydrogen; substituted or unsubstituted aliphatic; substituted or unsubstituted heteroaliphatic; substituted or unsubstituted aryl; substituted or unsubstituted heteroaryl; substituted or unsubstituted acyl; a resin; a suitable hydroxyl, amino or thiol protecting group; or two R^{B} groups together form a substituted or unsubstituted 5- to 6-membered heterocyclic or heteroaromatic ring;
each instance of R^{KL} is, independently, hydrogen; substituted or unsubstituted aliphatic; substituted or unsubstituted heteroaliphatic; substituted or unsubstituted aryl; substituted or unsubstituted heteroaryl; substituted or unsubstituted acyl; substituted or unsubstituted hydroxyl; substituted or unsubstituted thiol; substituted or unsubstituted amino; azido; cyano; isocyano; halo; or nitro;
each instance of R^{d} is, independently, hydrogen; substituted or unsubstituted aliphatic; substituted or unsubstituted heteroaliphatic; substituted or unsubstituted aryl; substituted or unsubstituted heteroaryl; substituted or unsubstituted acyl; or R^{d} is an amino protecting group;
each instance of R^{e} is, independently, a suitable amino acid side chain; hydrogen; substituted or unsubstituted aliphatic; substituted or unsubstituted heteroaliphatic; substituted or unsubstituted aryl; substituted or unsubstituted heteroaryl; substituted or unsubstituted acyl; substituted or unsubstituted hydroxyl; substituted or unsubstituted thiol; substituted or unsubstituted amino; cyano; isocyano; halo; or nitro;
each instance of G is, independently, a natural or unnatural amino acid or a group of the formula.

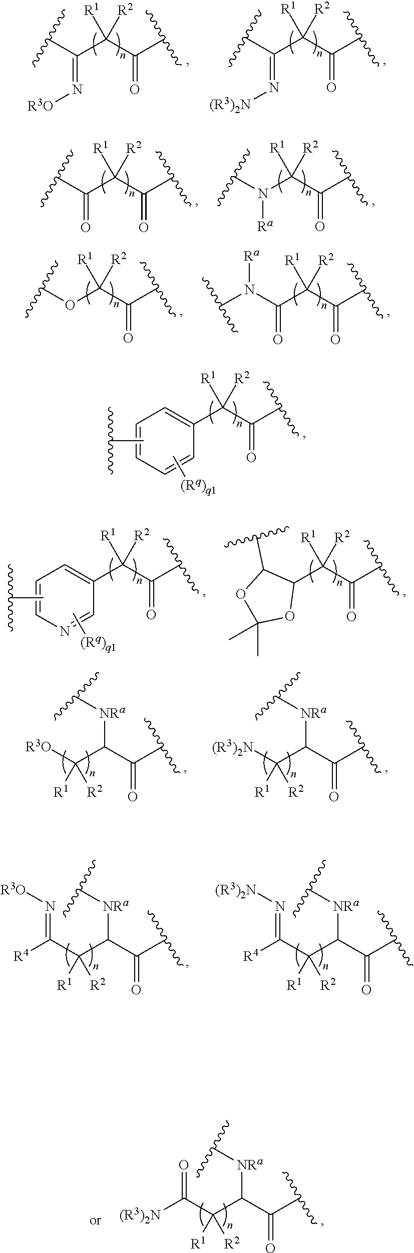

wherein:
n is 1, 2, or 3; and
each instance of R¹ and R² is independently hydrogen; substituted or unsubstituted aliphatic; substituted or unsubstituted heteroaliphatic; substituted or unsubstituted aryl; substituted or unsubstituted heteroaryl; substituted or unsubstituted acyl; substituted or unsubstituted hydroxyl; substituted or unsubstituted thiol; substituted or unsubstituted amino; or halo, or R¹ and R² are joined to form a carbocyclic or heterocyclic ring;
each instance of R³ and R⁴ is, independently, hydrogen; substituted or unsubstituted aliphatic; substituted or unsubstituted heteroaliphatic; a hydroxyl protecting group when attached to an oxygen atom, or an amino protecting group when attached to a nitrogen atom, or two R³ groups when attached to a nitrogen atom are joined to form a heterocyclic ring;
each instance of R^{q} is independently halogen, —CN, —NO₂, —N₃, optionally substituted alkyl, optionally substituted alkenyl, optionally substituted alkynyl, optionally substituted carbocyclyl, optionally substituted phenyl, optionally substituted heterocyclyl, optionally substituted heteroaryl, optionally substituted alkoxy, an optionally substituted amino group, or optionally substituted acyl;
q1 is 0, 1, 2, 3, or 4;
each instance of X_{AA} is, independently, a natural or unnatural amino acid;
j is, independently, an integer between 1 to 10, inclusive;
p is, independently, 1 or 2;
each instance of q is independently, 0, 1, or 2;
v is, independently, 0 or 1;
each instance of w and z is, independently, an integer between 0 and 100;
y is, independently, an integer of 1 to 8, inclusive; and
[symbol] corresponds to a single, double or triple bond.

In certain embodiments, the [symbol] corresponds to a double bond. In certain embodiments, the [symbol] corresponds to a triple bond.

In certain embodiments, the polypeptide of Formula (I) is of the formula:

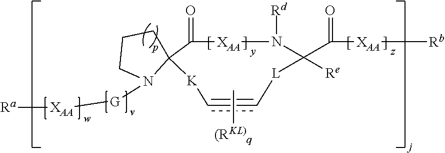

or a salt or stereoisomer thereof.

In certain embodiments, the polypeptide of Formula (I) is any one of the formulae:

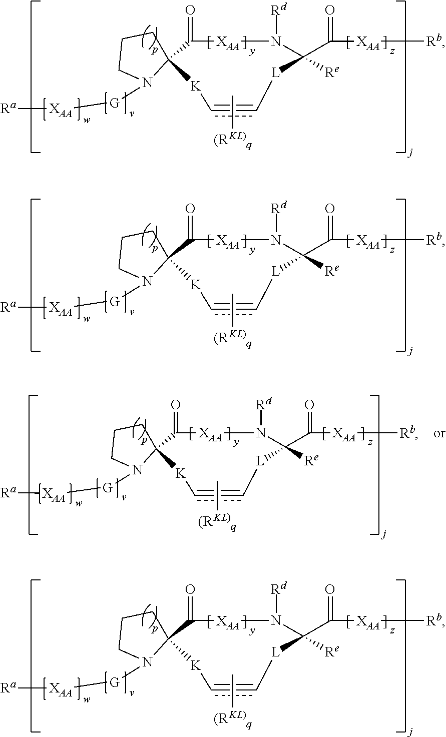

or a salt thereof.

In certain embodiments, the polypeptide of Formula (I) is any one of the formulae:

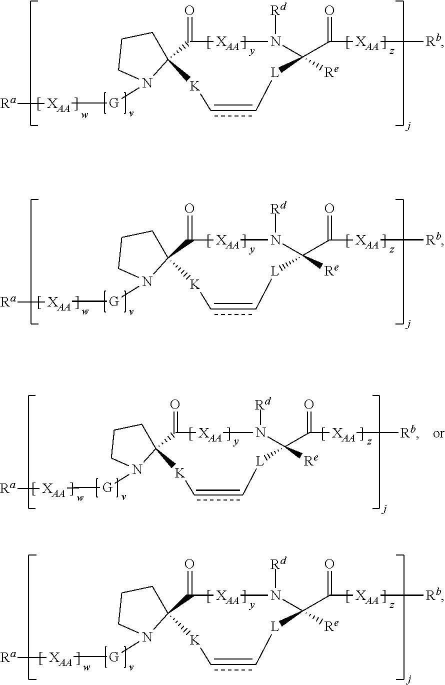

or a salt thereof.

In certain embodiments, the polypeptide of Formula (I) is of the formulae:

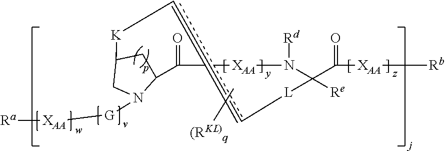

or a salt or stereoisomer thereof.

In certain embodiments, the polypeptide of Formula (I) is any one of the formula:

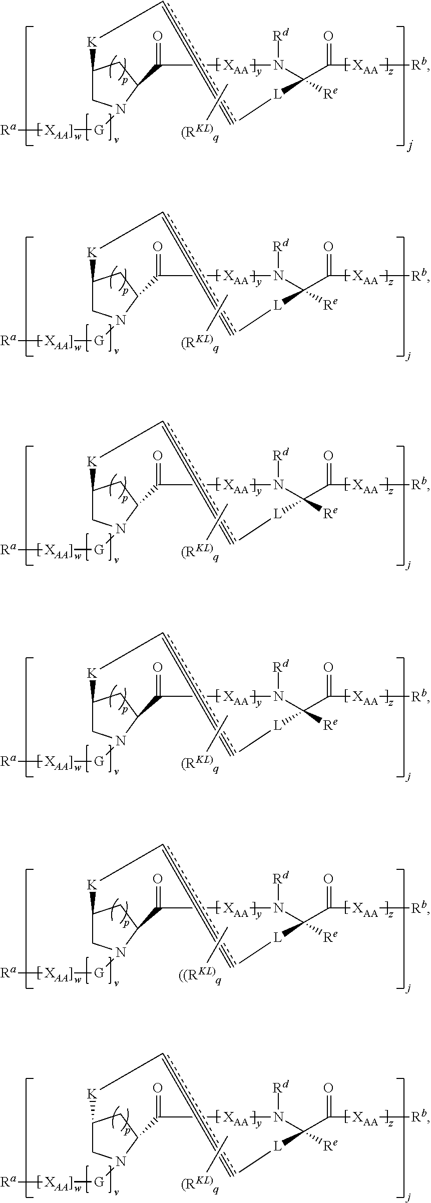

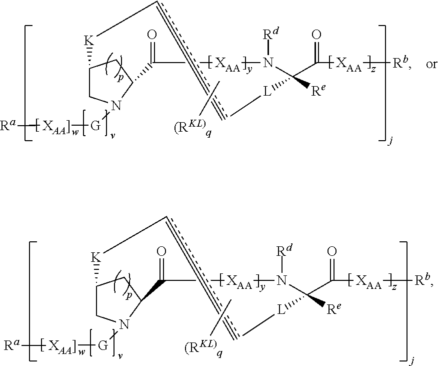

or a salt thereof.

In certain embodiments, the polypeptide of Formula (I) is any one of the formula:

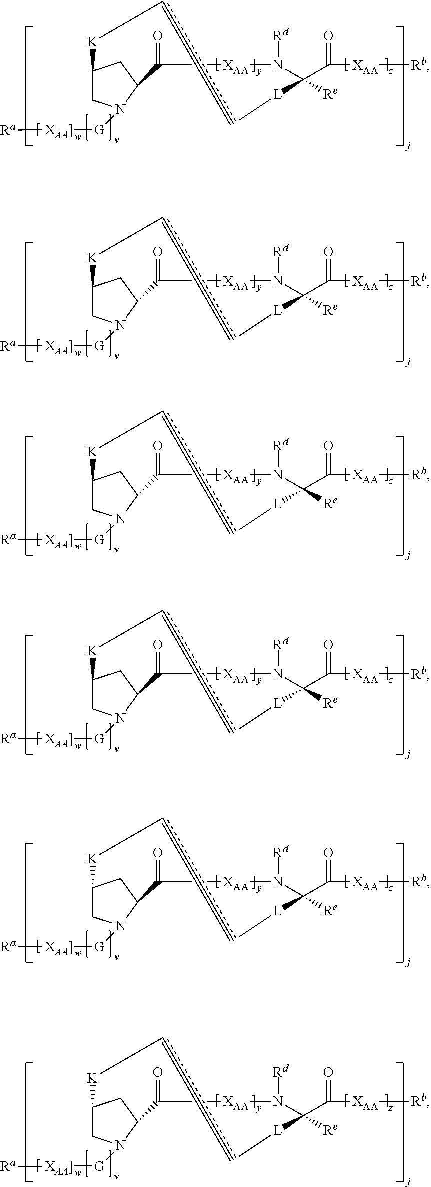

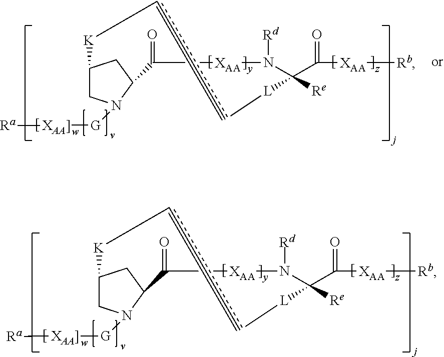

or a salt thereof.

In another aspect, provided is a precursor polypeptide of Formula (P-II):

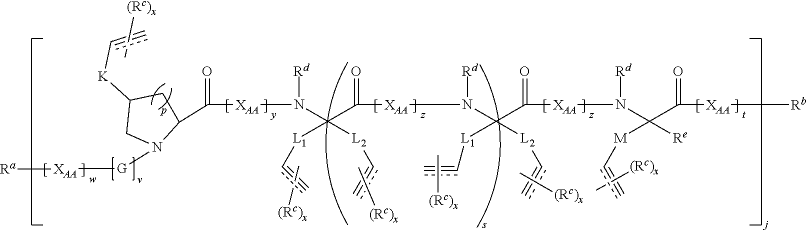

or a salt or stereoisomer thereof;
wherein:
each instance of K, L₁, L₂, and M, is, independently, a bond or a group consisting of one or more combinations of substituted or unsubstituted alkylene; substituted or unsubstituted alkenylene; substituted or unsubstituted alkynylene; substituted or unsubstituted heteroalkylene; substituted or unsubstituted heteroalkenylene; substituted or unsubstituted heteroalkynylene; substituted or unsubstituted heterocyclene, substituted or unsubstituted carbocyclene; substituted or unsubstituted arylene; and substituted or unsubstituted heteroarylene;
R^{a} is hydrogen, substituted or unsubstituted aliphatic; substituted or unsubstituted heteroaliphatic; substituted or unsubstituted aryl; substituted or unsubstituted heteroaryl; substituted or unsubstituted acyl; a resin; an amino protecting group; a label optionally joined by a linker, wherein the linker is a group consisting of one or more combinations of substituted or unsubstituted alkylene; substituted or unsubstituted alkenylene; substituted or unsubstituted alkynylene; substituted or unsubstituted heteroalkylene; substituted or unsubstituted heteroalkenylene; substituted or unsubstituted carbocyclene; substituted or unsubstituted heterocyclene; substituted or unsubstituted arylene; or substituted or unsubstituted heteroarylene;
R^{b} is, —R^{B}, —OR^{B}, —N(R^{B})₂, or —SR^{B}, wherein each instance of R^{B} is, independently, hydrogen; substituted or unsubstituted aliphatic; substituted or unsubstituted heteroaliphatic; substituted or unsubstituted aryl; substituted or unsubstituted heteroaryl; substituted or unsubstituted acyl; a resin; a suitable hydroxyl, amino or thiol protecting group; or two R^{B} groups together form a substituted or unsubstituted 5- to 6-membered heterocyclic or heteroaromatic ring;
each instance of R^{c}, is, independently, hydrogen; substituted or unsubstituted aliphatic; substituted or unsubstituted heteroaliphatic; substituted or unsubstituted aryl; substituted or unsubstituted heteroaryl; substituted or unsubstituted acyl; substituted or unsubstituted hydroxyl; substituted or unsubstituted thiol; substituted or unsubstituted amino; cyano; isocyano; halo; or nitro;
each instance of R^{d} is, independently, hydrogen; substituted or unsubstituted aliphatic; substituted or unsubstituted heteroaliphatic; substituted or unsubstituted aryl; substituted or unsubstituted heteroaryl; substituted or unsubstituted acyl; or R^{d} is an amino protecting group;
each instance of R^{e} is, independently, a suitable amino acid side chain; hydrogen; substituted or unsubstituted aliphatic; substituted or unsubstituted heteroaliphatic; substituted or unsubstituted aryl; substituted or unsubstituted heteroaryl; substituted or unsubstituted acyl; substituted or unsubstituted hydroxyl; substituted or unsubstituted thiol; substituted or unsubstituted amino; cyano; isocyano; halo; or nitro;
each instance of G is, independently, a natural or unnatural amino acid or a group of the formula:

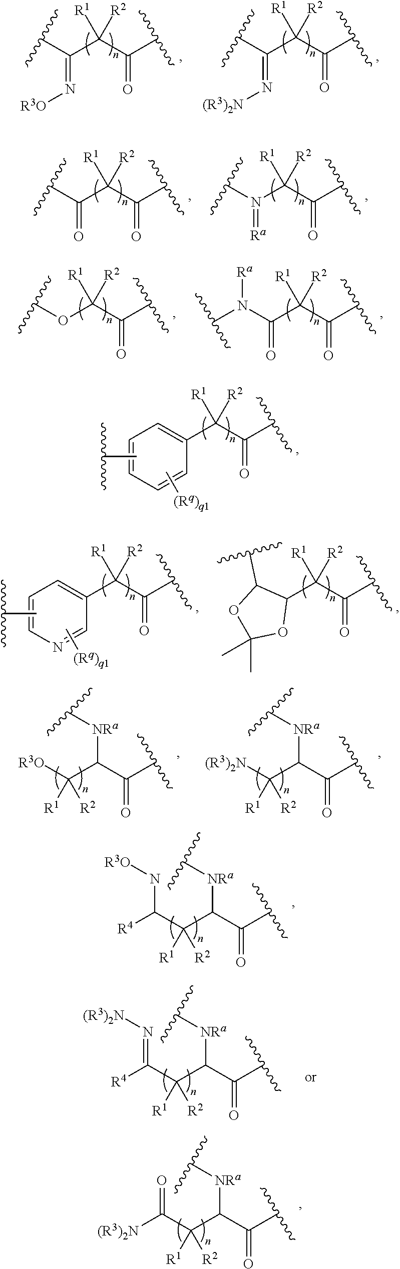

wherein:
n is 1, 2, or 3; and
each instance of R¹ and R² is independently hydrogen; substituted or unsubstituted aliphatic; substituted or unsubstituted heteroaliphatic; substituted or unsubstituted aryl; substituted or unsubstituted heteroaryl; substituted or unsubstituted acyl; substituted or unsubstituted hydroxyl; substituted or unsubstituted thiol; substituted or unsubstituted amino; or halo, or R¹ and R² are joined to form a carbocyclic or heterocyclic ring;
each instance of R³ and R⁴ is, independently, hydrogen; substituted or unsubstituted aliphatic; substituted or unsubstituted heteroaliphatic; a hydroxyl protecting group when attached to an oxygen atom, or an amino protecting group when attached to a nitrogen atom, or two R³ groups when attached to a nitrogen atom are joined to form a heterocyclic ring;
each instance of R^{q} is independently halogen, —CN, —NO₂, —N₃, optionally substituted alkyl, optionally substituted alkenyl, optionally substituted alkynyl, optionally substituted carbocyclyl, optionally substituted phenyl, optionally substituted heterocyclyl, optionally substituted heteroaryl, optionally substituted alkoxy, an optionally substituted amino group, or optionally substituted acyl;
q1 is 0, 1, 2, 3, or 4;
each instance of X_{AA} is, independently, a natural or unnatural amino acid;
j is, independently, an integer between 1 to 10, inclusive;
p is, independently, 1 or 2;
v is, independently, 0 or 1;
s is 0, 1, or 2;
each instance of t, w and z is, independently, an integer between 0 and 100, inclusive;
each instance of x is, independently, 0, 1, 2, or 3;
y is, independently 1, 2, 3, or 4; and
[symbol] corresponds to a double or triple bond.

In certain embodiments, the [symbol] corresponds to a double bond. In certain embodiments, the [symbol] corresponds to a triple bond.

In certain embodiments, the polypeptide of Formula (P-II) is of the formula:

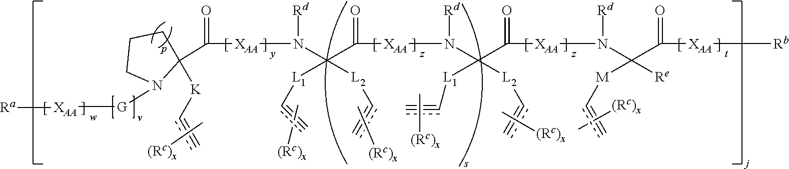

or a salt or stereoisomer thereof.

In certain embodiments, the polypeptide of Formula (P-II) is any one of the formula:

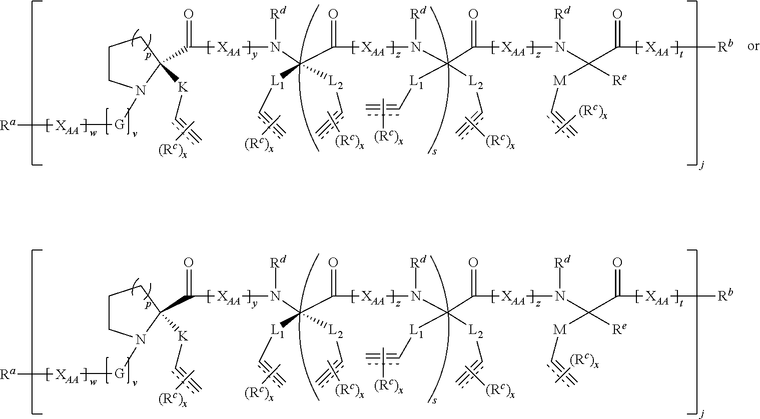

or salt or stereoisomer thereof.

In certain embodiments, the polypeptide of Formula (P-II) is any one of the formula:

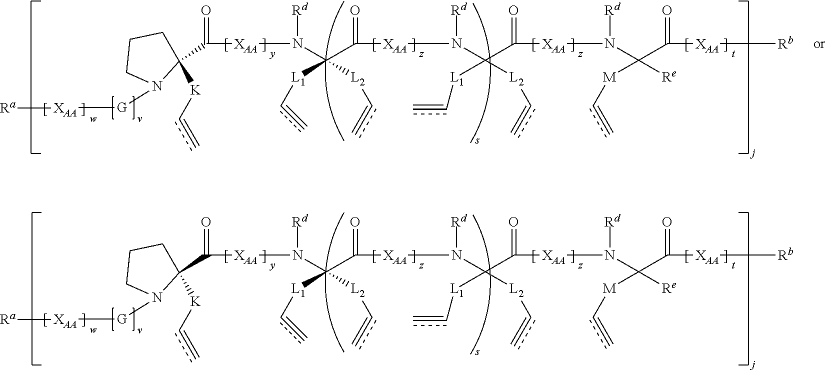

or salt or stereoisomer thereof.

In certain embodiments, the polypeptide of Formula (P-II) is of the formula:

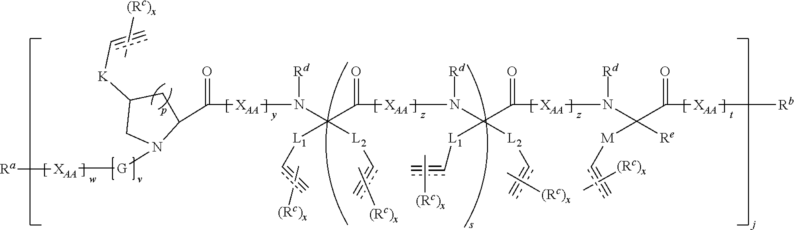

or a salt or stereoisomer thereof.

In certain embodiments, the polypeptide of Formula (II) is any one of the formula:

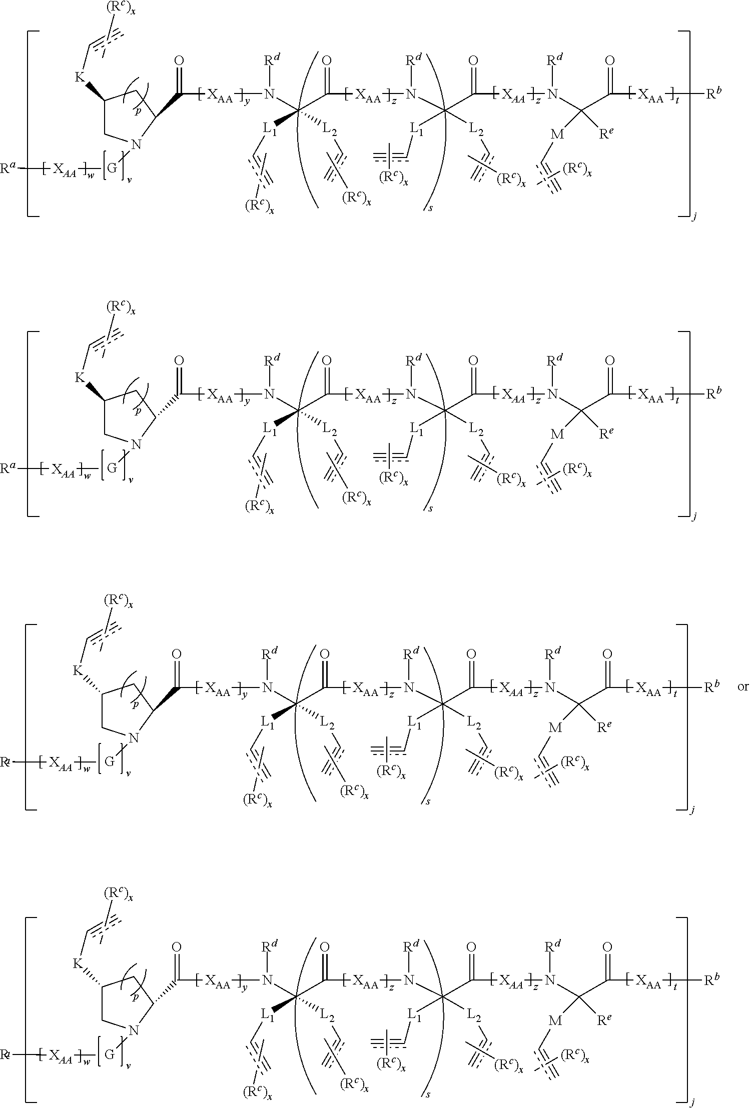

or a salt or stereoisomer thereof.

In certain embodiments, the polypeptide of Formula (II) is any one of the formula:

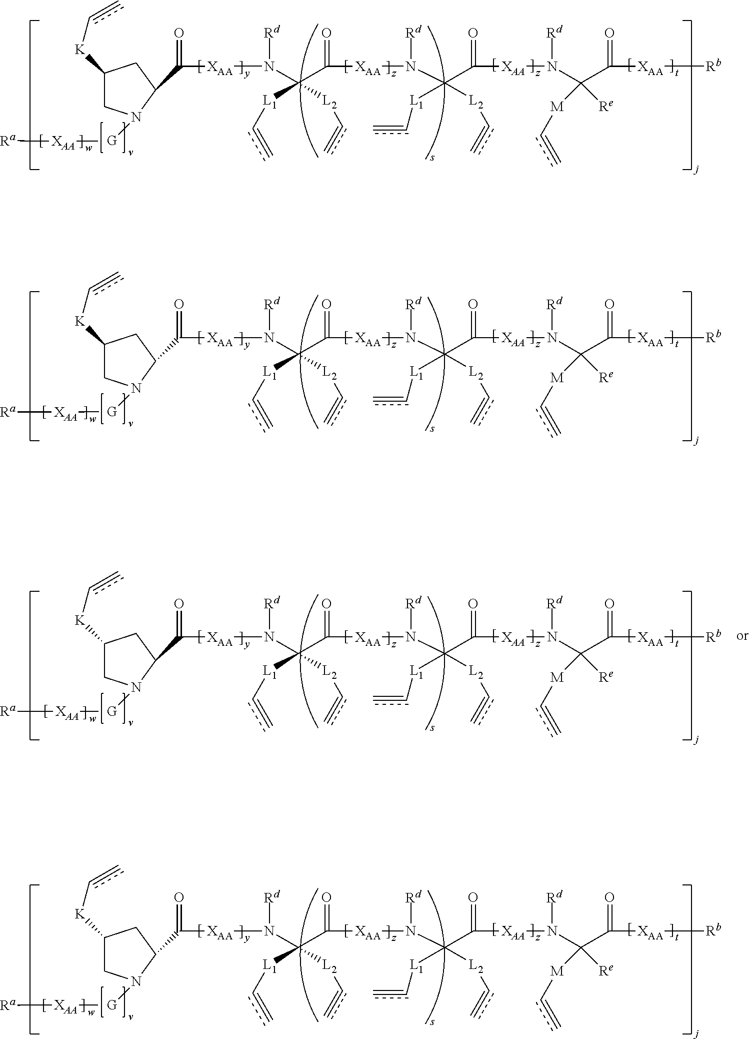

or a salt or stereoisomer thereof.

Furthermore, in certain embodiments, the precursor polypeptide of Formula (P-II), upon contact with a ring closing methathesis catalyst, generates a stitched polypeptide of Formula (II):

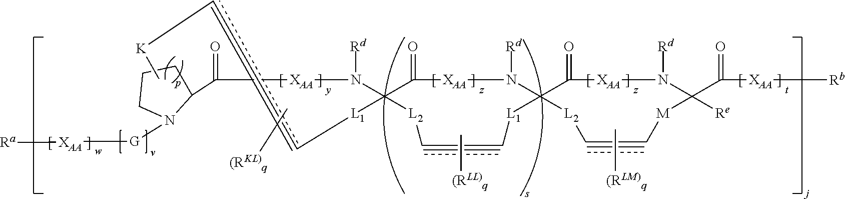

or a salt or stereoisomer thereof.
wherein:
each instance of K, M, L₁, and L₂, is independently, a bond or a group consisting of one or more combinations of substituted or unsubstituted alkylene; substituted or unsubstituted alkenylene; substituted or unsubstituted alkynylene; substituted or unsubstituted heteroalkylene; substituted or unsubstituted heteroalkenylene; substituted or unsubstituted heteroalkynylene; substituted or unsubstituted heterocyclene, substituted or unsubstituted carbocyclene, substituted or unsubstituted arylene; substituted or unsubstituted heteroarylene;
R^{a} is hydrogen; substituted or unsubstituted aliphatic; substituted or unsubstituted heteroaliphatic; substituted or unsubstituted aryl; substituted or unsubstituted heteroaryl; substituted or unsubstituted acyl; a resin; an amino protecting group; a label optionally joined by a linker, wherein the linker is a group consisting of one or more combinations of substituted or unsubstituted alkylene; substituted or unsubstituted alkenylene; substituted or unsubstituted alkynylene; substituted or unsubstituted heteroalkylene; substituted or unsubstituted heteroalkenylene; substituted or unsubstituted carbocyclene; substituted or unsubstituted heterocyclene; substituted or unsubstituted arylene; or substituted or unsubstituted heteroarylene;
R^{b} is, —R^{B}, —OR^{B}, —N(R^{B})₂, or —SR^{B}, wherein each instance of R^{B} is, independently, hydrogen; substituted or unsubstituted aliphatic; substituted or unsubstituted heteroaliphatic; substituted or unsubstituted aryl; substituted or unsubstituted heteroaryl; substituted or unsubstituted acyl; a resin; a suitable hydroxyl, amino or thiol protecting group; or two R^{B} groups together form a substituted or unsubstituted 5- to 6-membered heterocyclic or heteroaromatic ring;
each instance of R^{c}, is, independently, hydrogen; substituted or unsubstituted aliphatic; substituted or unsubstituted heteroaliphatic; substituted or unsubstituted aryl; substituted or unsubstituted heteroaryl; substituted or unsubstituted acyl; substituted or unsubstituted hydroxyl; substituted or unsubstituted thiol; substituted or unsubstituted amino; cyano; isocyano; halo; or nitro;
each instance of R^{d} is, independently, hydrogen; substituted or unsubstituted aliphatic; substituted or unsubstituted heteroaliphatic; substituted or unsubstituted aryl; substituted or unsubstituted heteroaryl; substituted or unsubstituted acyl; or R^{d} is an amino protecting group;
each instance of R^{e} is, independently, a suitable amino acid side chain; hydrogen; substituted or unsubstituted aliphatic; substituted or unsubstituted heteroaliphatic; substituted or unsubstituted aryl; substituted or unsubstituted heteroaryl; substituted or unsubstituted acyl; substituted or unsubstituted hydroxyl; substituted or unsubstituted thiol; substituted or unsubstituted amino; cyano; isocyano; halo; or nitro;
each instance of R^{KL}, R^{LL}, and R^{LM}, is, independently, hydrogen; substituted or unsubstituted aliphatic; substituted or unsubstituted heteroaliphatic; substituted or unsubstituted aryl; substituted or unsubstituted heteroaryl; substituted or unsubstituted acyl; substituted or unsubstituted hydroxyl; substituted or unsubstituted thiol; substituted or unsubstituted amino; azido; cyano; isocyano; halo; nitro;
each instance of G is, independently, a natural or unnatural amino acid or a group of the formula:

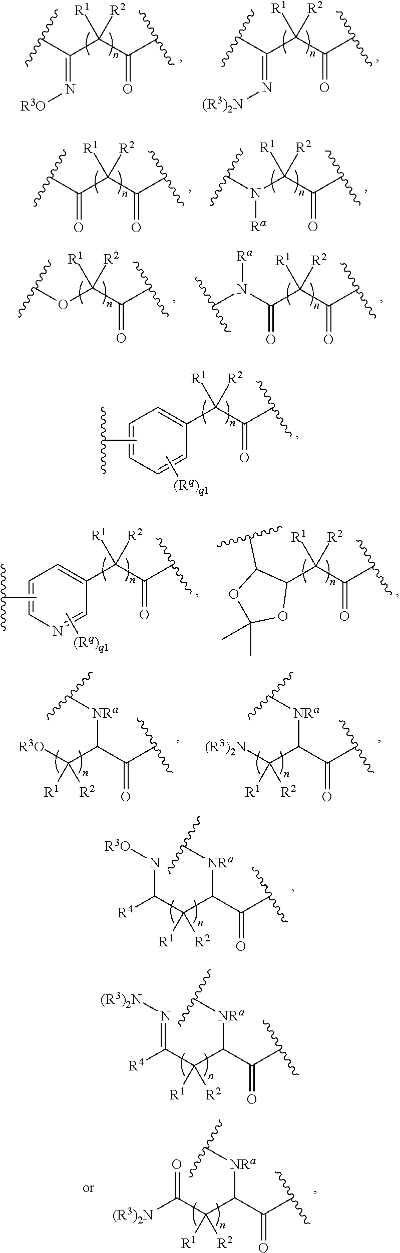

wherein:
n is 1, 2, or 3; and
each instance of R¹ and R² is independently hydrogen; substituted or unsubstituted aliphatic; substituted or unsubstituted heteroaliphatic; substituted or unsubstituted aryl; substituted or unsubstituted heteroaryl; substituted or unsubstituted acyl; substituted or unsubstituted hydroxyl; substituted or unsubstituted thiol; substituted or unsubstituted amino; or halo, or R¹ and R² are joined to form a carbocyclic or heterocyclic ring;
each instance of R³ and R⁴ is, independently, hydrogen; substituted or unsubstituted aliphatic; substituted or unsubstituted heteroaliphatic; a hydroxyl protecting group when attached to an oxygen atom, or an amino protecting group when attached to a nitrogen atom, or two R³ groups when attached to a nitrogen atom are joined to form a heterocyclic ring;
each instance of R^{q} is independently halogen, —CN, —NO₂, —N₃, optionally substituted alkyl, optionally substituted alkenyl, optionally substituted alkynyl, optionally substituted carbocyclyl, optionally substituted phenyl, optionally substituted heterocyclyl, optionally substituted heteroaryl, optionally substituted alkoxy, an optionally substituted amino group, or optionally substituted acyl;
q1 is 0, 1, 2, 3, or 4;
each instance of X_{AA} is, independently, a natural or unnatural amino acid;
j is, independently, an integer between 1 to 10, inclusive;
p is, independently, 1 or 2;
each instance of q is independently, 0, 1 or 2;
v is, independently, an integer between 0 to 1;
s is 0, 1, or 2;
each instance of t, w and z is, independently, an integer between 0 and 100;
y is, independently, an integer of 1 to 8, inclusive; and
[symbol] corresponds to a single, double or triple bond.

In certain embodiments, the [symbol] corresponds to a double bond. In certain embodiments, the [symbol] corresponds to a triple bond.

In certain embodiments, the polypeptide of Formula (II) is of the formula:

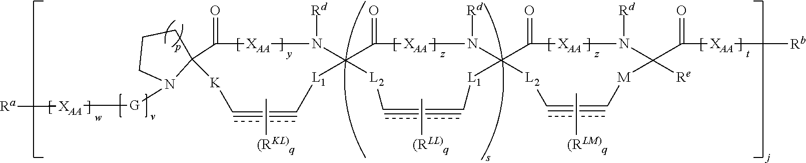

or a salt or stereoisomer thereof.

In certain embodiments, the polypeptide of Formula (II) is any one of the formula:

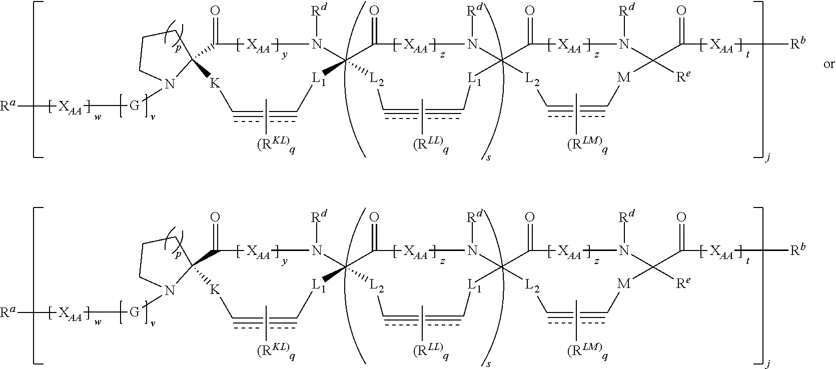

or a salt or stereoisomer thereof.

In certain embodiments, the polypeptide of Formula (II) is any one of the formula:

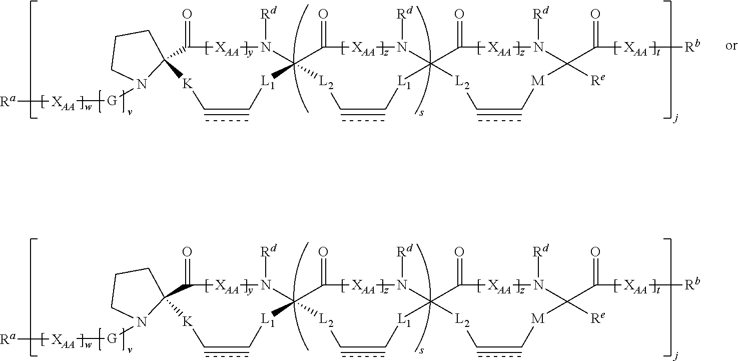

or a salt or stereoisomer thereof.

In certain embodiments, the polypeptide of Formula (II) is of the formula:

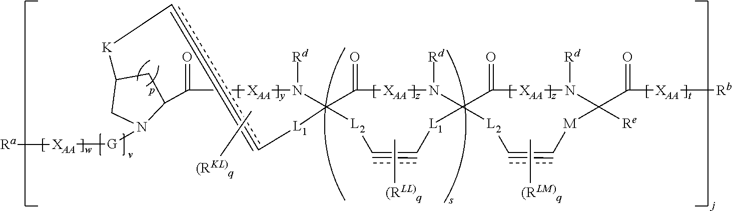

or a salt or stereoisomer thereof.

In certain embodiments, the polypeptide of Formula (II) is any one of the formula:

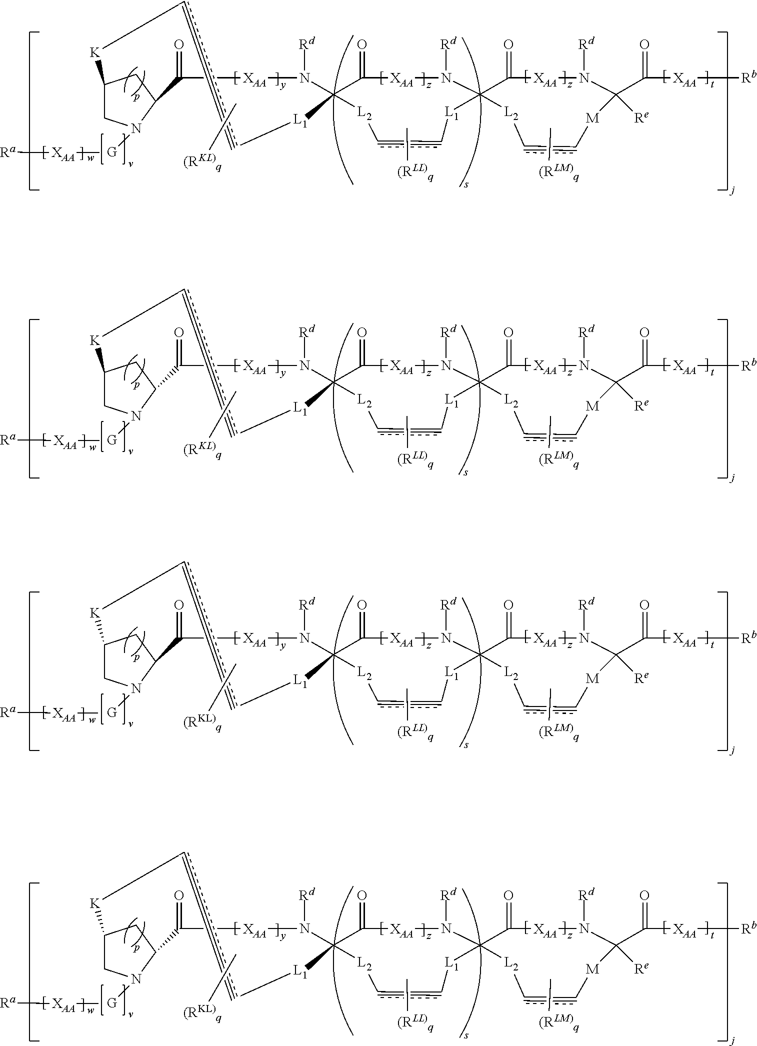

or a salt or stereoisomer thereof.

In certain embodiments, the polypeptide of Formula (II) is any one of the formula

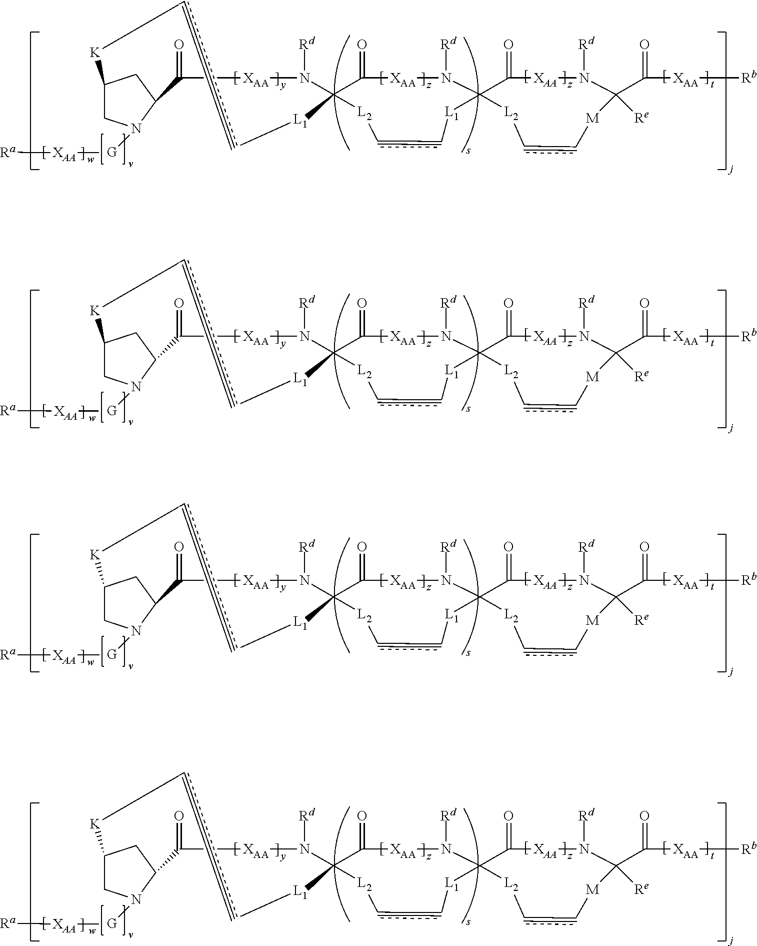

or a salt or stereoisomer thereof.

In yet another aspect, provided are compounds useful in the preparation of the precursor polypeptides which include, but are not limited to, compounds of Formula (III):

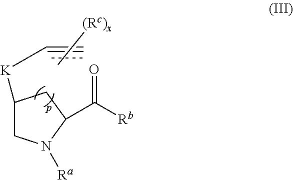

or salts or stereoisomers thereof; wherein:
p is 1 or 2;
K is a bond or a group consisting of one or more combinations of substituted or unsubstituted alkylene; substituted or unsubstituted alkenylene; substituted or unsubstituted alkynylene; substituted or unsubstituted heteroalkylene; substituted or unsubstituted heteroalkenylene; substituted or unsubstituted heteroalkynylene; substituted or unsubstituted heterocyclene, substituted or unsubstituted carbocyclene, substituted or unsubstituted arylene; substituted or unsubstituted heteroarylene;
R^{a} is hydrogen, substituted or unsubstituted aliphatic; substituted or unsubstituted heteroaliphatic; substituted or unsubstituted aryl; substituted or unsubstituted heteroaryl; substituted or unsubstituted acyl; a resin; an amino protecting group; or a label optionally joined by a linker, wherein the linker is a group consisting of one or more combinations of substituted or unsubstituted alkylene; substituted or unsubstituted alkenylene; substituted or unsubstituted alkynylene; substituted or unsubstituted heteroalkylene; substituted or unsubstituted heteroalkenylene; substituted or unsubstituted carbocyclene; substituted or unsubstituted heterocyclene; substituted or unsubstituted arylene; or substituted or unsubstituted heteroarylene;
R^{b} is, —R^{B}, —OR^{B}, —N(R^{B})₂, or —SR^{B}, wherein each instance of R^{B} is, independently, hydrogen; substituted or unsubstituted aliphatic; substituted or unsubstituted heteroaliphatic; substituted or unsubstituted aryl; substituted or unsubstituted heteroaryl; substituted or unsubstituted acyl; a resin; a suitable hydroxyl, amino or thiol protecting group; or two R^{B} groups together form a substituted or unsubstituted 5- to 6-membered heterocyclic or heteroaromatic ring;
each instance of R^{c}, is, independently, hydrogen; substituted or unsubstituted aliphatic; substituted or unsubstituted heteroaliphatic; substituted or unsubstituted aryl; substituted or unsubstituted heteroaryl; substituted or unsubstituted acyl; substituted or unsubstituted hydroxyl; substituted or unsubstituted thiol; substituted or unsubstituted amino; cyano; isocyano; halo; or nitro;
x is 0, 1, 2, or 3; and
[symbol] corresponds to a single, double or triple bond.

In certain embodiments, the [symbol] corresponds to a double bond. In certain embodiments, the [symbol] corresponds to a triple bond.

In certain embodiments, the compound of Formula (III) is of the formula:

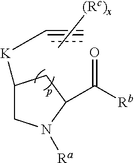

or salt or stereoisomer thereof.

In certain embodiments, the compound of Formula (III) is any one of the formula:

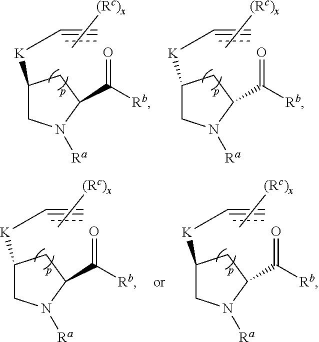

or salt thereof.

In certain embodiments, the compound of Formula (III) is any one of the formula:

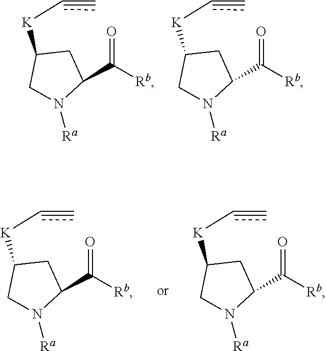

or salt thereof.

In certain embodiments, the compound of Formula (III) is of the formula:

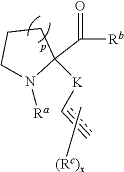

or salt or stereoisomer thereof.

In certain embodiments, the compound of Formula (III) is any one of the formula:

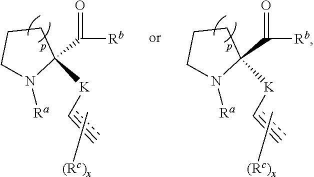

or salt thereof.

In certain embodiments, the compound of Formula (III) is any one of the formula:

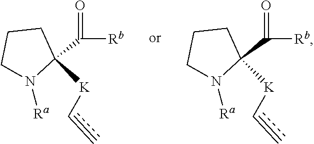

or salt thereof.
Groups K, L, L₁, L₂, and M

As generally defined above, each instance of K, L, L₁, L₂, and M is, independently, a bond or a group consisting of one or more combinations of substituted or unsubstituted alkylene; substituted or unsubstituted alkenylene; substituted or unsubstituted alkynylene; substituted or unsubstituted heteroalkylene; substituted or unsubstituted heteroalkenylene; substituted or unsubstituted heteroalkynylene; substituted or unsubstituted heterocyclene, substituted or unsubstituted carbocyclene, substituted or unsubstituted arylene; substituted or unsubstituted heteroarylene.

As used herein, reference to a group consisting of “one or more combinations” refers to a group comprising 1, 2, 3, 4 or more combinations of the recited divinyl moieties. For example, the group may consist of an alkylene attached to a heteroalkylene, which may be further optionally attached to another alkylene. As used herein “at least one instance” refers to 1, 2, 3, or 4 instances of the recited moiety.

In certain embodiments, K is a bond.

In certain embodiments, K is a group consisting of one or more combinations of substituted or unsubstituted alkylene; substituted or unsubstituted alkenylene; substituted or unsubstituted alkynylene; substituted or unsubstituted heteroalkylene; substituted or unsubstituted heteroalkenylene; substituted or unsubstituted heteroalkynylene; substituted or unsubstituted heterocyclene, substituted or unsubstituted carbocyclene, substituted or unsubstituted arylene; substituted or unsubstituted heteroarylene.

In certain embodiments, K is a group which comprises at least one instance of substituted or unsubstituted alkylene, e.g., substituted or unsubstituted C₁₋₆alkylene, substituted or unsubstituted C₁₋₂alkylene, substituted or unsubstituted C₂₋₃alkylene, substituted or unsubstituted C₃₋₄alkylene, substituted or unsubstituted C₄₋₅alkylene, or substituted or unsubstituted C₅₋₄alkylene. Exemplary alkylene groups include unsubstituted alkylene groups such as methylene —CH₂—, ethylene —(CH₂)₂—, n-propylene —(CH₂)₃—, n-butylene —(CH₂)₄—, n-pentylene —(CH₂)₅—, and n-hexylene —(CH₂)₆—. In certain embodiments, K is —CH₂—. In certain embodiments, K is —(CH₂)₂—. In certain embodiments, K is —(CH₂)₃—.

In certain embodiments, K is a group which comprises at least one instance of substituted or unsubstituted alkenylene, e.g., substituted or unsubstituted C₂₋₆alkenylene, substituted or unsubstituted C₂₋₃alkenylene, substituted or unsubstituted C₃₋₄alkenylene, substituted or unsubstituted C₄₋₅alkenylene, or substituted or unsubstituted C₅₋₆alkenylene.

In certain embodiments, K is a group which comprises at least one instance of substituted or unsubstituted alkynylene, e.g., substituted or unsubstituted C₂₋₆alkynylene, substituted or unsubstituted C₂₋₃alkynylene, substituted or unsubstituted C₃₋₄alkynylene, substituted or unsubstituted C₄₋₅alkynylene, or substituted or unsubstituted C₅₋₆alkynylene.

In certain embodiments, K is a group which comprises at least one instance of substituted or unsubstituted heteroalkylene, e.g., substituted or unsubstituted heteroC₁₋₆alkylene, substituted or unsubstituted heteroC₁₋₂alkylene, substituted or unsubstituted heteroC₂₋₃alkylene, substituted or unsubstituted heteroC₃₋₄alkylene, substituted or unsubstituted heteroC₄₋₅alkylene, or substituted or unsubstituted heteroC₅₋₆alkylene. Exemplary heteroalkylene groups include unsubstituted alkylene groups such as —(CH₂)₂—O(CH₂)₂—, —OCH₂—, —O(CH₂)₂—, —O(CH₂)₃—, —O(CH₂)₄—, —O(CH₂)₅—, and —O(CH₂)₆—. In certain embodiments, K is —CH₂O—, wherein O is linked to the heterocyclyl with nitrogen and CH₂ is linked to “ [symbol] ” In certain embodiments, K is —(CH₂)₂O—, wherein O is linked to the heterocyclyl with nitrogen and CH₂ is linked to “ [symbol] ”. In certain embodiments, K is —(CH₂)₃O—, wherein O is linked to the heterocyclyl with nitrogen and CH₂ is linked to “ [symbol] .”

In certain embodiments, K is a group which comprises at least one instance of substituted or unsubstituted heteroalkenylene, e.g., substituted or unsubstituted heteroC₂₋₆alkenylene, substituted or unsubstituted heteroC₂₋₃alkenylene, substituted or unsubstituted heteroC₃₋₄alkenylene, substituted or unsubstituted heteroC₄₋₅alkenylene, or substituted or unsubstituted heteroC₅₋₆alkenylene.

In certain embodiments, K is a group which comprises at least one instance of substituted or unsubstituted heteroalkynylene, e.g., substituted or unsubstituted heteroC₂₋₆alkynylene, substituted or unsubstituted heteroC₂₋₃alkynylene, substituted or unsubstituted heteroC₃₋₄alkynylene, substituted or unsubstituted heteroC₄₋₅alkynylene, or substituted or unsubstituted heteroC₅₋₆alkynylene.

In certain embodiments, K is a group which comprises at least one instance of substituted or unsubstituted carbocyclylene, e.g., substituted or unsubstituted C₃₋₆carbocyclylene, substituted or unsubstituted C₃₋₄carbocyclylene, substituted or unsubstituted C₄₋₅ carbocyclylene, or substituted or unsubstituted C₅₋₆ carbocyclylene.

In certain embodiments, K is a group which comprises at least one instance of substituted or unsubstituted heterocyclylene, e.g., substituted or unsubstituted C₃₋₆ heterocyclylene, substituted or unsubstituted C₃₋₄ heterocyclylene, substituted or unsubstituted C₄₋₅ heterocyclylene, or substituted or unsubstituted C₅₋₆ heterocyclylene.

In certain embodiments, K is a group which comprises at least one instance of substituted or unsubstituted arylene, e.g., substituted or unsubstituted phenylene.

In certain embodiments, K is a group which comprises at least one instance of substituted or unsubstituted heteroarylene, e.g., substituted or unsubstituted 5- to 6-membered heteroarylene.

In certain embodiments, L is a bond.

In certain embodiments, L is a group consisting of one or more combinations of substituted or unsubstituted alkylene; substituted or unsubstituted alkenylene; substituted or unsubstituted alkynylene; substituted or unsubstituted heteroalkylene; substituted or unsubstituted heteroalkenylene; substituted or unsubstituted heteroalkynylene; substituted or unsubstituted heterocyclene, substituted or unsubstituted carbocyclene, substituted or unsubstituted arylene; substituted or unsubstituted heteroarylene.

In certain embodiments, L is a group which comprises at least one instance of substituted or unsubstituted alkylene, e.g., substituted or unsubstituted C₁₋₆alkylene, substituted or unsubstituted C₁₋₂alkylene, substituted or unsubstituted C₂₋₃alkylene, substituted or unsubstituted C₃₋₄alkylene, substituted or unsubstituted C₄₋₅alkylene, or substituted or unsubstituted C₅₋₆alkylene. Exemplary alkylene groups include unsubstituted alkylene groups such as methylene —CH₂—, ethylene —(CH₂)₂—, n-propylene —(CH₂)₃—, n-butylene —(CH₂)₄—, n-pentylene —(CH₂)₅—, and n-hexylene —(CH₂)₆—. In certain embodiments, L is —CH₂—. In certain embodiments, L is —(CH₂)₂—. In certain embodiments, L is —(CH₂)₃—. In certain embodiments, L is —(CH₂)₄—. In certain embodiments, L is —(CH₂)₅—. In certain embodiments, L is —(CH₂)₆—.

In certain embodiments, L is a group which comprises at least one instance of substituted or unsubstituted alkenylene, e.g., substituted or unsubstituted C₂₋₆alkenylene, substituted or unsubstituted C₂₋₃alkenylene, substituted or unsubstituted C₃₋₄alkenylene, substituted or unsubstituted C₄₋₅alkenylene, or substituted or unsubstituted C₅₋₆alkenylene.

In certain embodiments, L is a group which comprises at least one instance of substituted or unsubstituted alkynylene, e.g., substituted or unsubstituted C₂₋₆alkynylene, substituted or unsubstituted C₂₋₃alkynylene, substituted or unsubstituted C₃₋₄alkynylene, substituted or unsubstituted C₄₋₅alkynylene, or substituted or unsubstituted C₅₋₆alkynylene.

In certain embodiments, L is a group which comprises at least one instance of substituted or unsubstituted heteroalkylene, e.g., substituted or unsubstituted heteroC₁₋₆alkylene, substituted or unsubstituted heteroC₅₋₂alkylene, substituted or unsubstituted heteroC₂₋₃alkylene, substituted or unsubstituted heteroC₃₋₄alkylene, substituted or unsubstituted heteroC₄₋₅alkylene, or substituted or unsubstituted heteroC₅₋₆alkylene. Exemplary heteroalkylene groups include unsubstituted alkylene groups such as —(CH₂)₂—O(CH₂)₂—, —OCH₂—, —O(CH₂)₂—, —O(CH₂)₃—, —O(CH₂)₄—, —O(CH₂)₅—, and —O(CH₂)₆—.

In certain embodiments, L is a group which comprises at least one instance of substituted or unsubstituted heteroalkenylene, e.g., substituted or unsubstituted heteroC₂₋₆alkenylene, substituted or unsubstituted heteroC₂₋₃alkenylene, substituted or unsubstituted heteroC₃₋₄alkenylene, substituted or unsubstituted heteroC₄₋₅alkenylene, or substituted or unsubstituted heteroC₅₋₆alkenylene.

In certain embodiments, L is a group which comprises at least one instance of substituted or unsubstituted heteroalkynylene, e.g., substituted or unsubstituted heteroC₂₋₆alkynylene, substituted or unsubstituted heteroC₂₋₃alkynylene, substituted or unsubstituted heteroC₃₋₄alkynylene, substituted or unsubstituted heteroC₄₋₅alkynylene, or substituted or unsubstituted heteroC₅₋₆alkynylene.

In certain embodiments, L is a group which comprises at least one instance of substituted or unsubstituted carbocyclylene, e.g., substituted or unsubstituted C₃₋₆carbocyclylene, substituted or unsubstituted C₃₋₄carbocyclylene, substituted or unsubstituted C₄₋₅ carbocyclylene, or substituted or unsubstituted C₅₋₆ carbocyclylene.

In certain embodiments, L is a group which comprises at least one instance of substituted or unsubstituted heterocyclylene, e.g., substituted or unsubstituted C₃₋₆ heterocyclylene, substituted or unsubstituted C₃₋₄ heterocyclylene, substituted or unsubstituted C₄₋₅ heterocyclylene, or substituted or unsubstituted C₅₋₆ heterocyclylene.

In certain embodiments, L is a group which comprises at least one instance of substituted or unsubstituted arylene, e.g., substituted or unsubstituted phenylene.

In certain embodiments, L is a group which comprises at least one instance of substituted or unsubstituted heteroarylene, e.g., substituted or unsubstituted 5- to 6-membered heteroarylene.

In certain embodiments, L₁ is a bond.

In certain embodiments, L₁ is a group consisting of one or more combinations of substituted or unsubstituted alkylene; substituted or unsubstituted alkenylene; substituted or unsubstituted alkynylene; substituted or unsubstituted heteroalkylene; substituted or unsubstituted heteroalkenylene; substituted or unsubstituted heteroalkynylene; substituted or unsubstituted heterocyclene, substituted or unsubstituted carbocyclene, substituted or unsubstituted arylene; substituted or unsubstituted heteroarylene.

In certain embodiments, L₁ is a group which comprises at least one instance of substituted or unsubstituted alkylene, e.g., substituted or unsubstituted C₁₋₆alkylene, substituted or unsubstituted C₁₋₂alkylene, substituted or unsubstituted C₂₋₃alkylene, substituted or unsubstituted C₃₋₄alkylene, substituted or unsubstituted C₄₋₅alkylene, or substituted or unsubstituted C₅₋₆alkylene. Exemplary alkylene groups include unsubstituted alkylene groups such as methylene —CH₂—, ethylene —(CH₂)₂—, n-propylene —(CH₂)₃—, n-butylene —(CH₂)₄—, n-pentylene —(CH₂)₅—, and n-hexylene —(CH₂)₆—. In certain embodiments, L₁ is —CH₂—. In certain embodiments, L₁ is —(CH₂)₂—. In certain embodiments, L₁ is —(CH₂)₃—. In certain embodiments, L₁ is —(CH₂)₄—. In certain embodiments, L₁ is —(CH₂)₃—. In certain embodiments. L₁ is —(CH₂)₆—.

In certain embodiments, L₁ is a group which comprises at least one instance of substituted or unsubstituted alkenylene, e.g., substituted or unsubstituted C₂₋₆alkenylene, substituted or unsubstituted C₂₋₃alkenylene, substituted or unsubstituted C₃₋₄alkenylene, substituted or unsubstituted C₄₋₅alkenylene, or substituted or unsubstituted C₅₋₆alkenylene.

In certain embodiments, L₁ is a group which comprises at least one instance of substituted or unsubstituted alkynylene, e.g., substituted or unsubstituted C₂₋₆alkynylene, substituted or unsubstituted C₂₋₃alkynylene, substituted or unsubstituted C₃₋₄alkynylene, substituted or unsubstituted C₄₋₅alkynylene, or substituted or unsubstituted C₅₋₆alkynylene.

In certain embodiments, L₁ is a group which comprises at least one instance of substituted or unsubstituted heteroalkylene, e.g., substituted or unsubstituted heteroC₁₋₆alkylene, substituted or unsubstituted heteroC₁₋₂alkylene, substituted or unsubstituted heteroC₂₋₃alkylene, substituted or unsubstituted heteroC₃₋₄alkylene, substituted or unsubstituted heteroC₄₋₅alkylene, or substituted or unsubstituted heteroC₅₋₆alkylene. Exemplary heteroalkylene groups include unsubstituted alkylene groups such as —(CH₂)₂—O(CH₂)₂—, —OCH₂—, —O(CH₂)₂—, —O(CH₂)₃—, —O(CH₂)₄—, —O(CH₂)₅—, and —O(CH₂)₆—.

In certain embodiments, L₁ is a group which comprises at least one instance of substituted or unsubstituted heteroalkenylene, e.g., substituted or unsubstituted heteroC₂₋₆alkenylene, substituted or unsubstituted heteroC₂₋₃alkenylene, substituted or unsubstituted heteroC₃₋₄alkenylene, substituted or unsubstituted heteroC₄₋₅alkenylene, or substituted or unsubstituted heteroC₅₋₆alkenylene.

In certain embodiments, L₁ is a group which comprises at least one instance of substituted or unsubstituted heteroalkynylene, e.g., substituted or unsubstituted heteroC₂₋₆alkynylene, substituted or unsubstituted heteroC₂₋₃alkynylene, substituted or unsubstituted heteroC₃₋₄alkynylene, substituted or unsubstituted heteroC₄₋₅alkynylene, or substituted or unsubstituted heteroC₅₋₆alkynylene.

In certain embodiments, L₁ is a group which comprises at least one instance of substituted or unsubstituted carbocyclylene, e.g., substituted or unsubstituted C₃₋₆carbocyclylene, substituted or unsubstituted C₃₋₄carbocyclylene, substituted or unsubstituted C₄₋₅ carbocyclylene, or substituted or unsubstituted C₅₋₆ carbocyclylene.

In certain embodiments, L₁ is a group which comprises at least one instance of substituted or unsubstituted heterocyclylene, e.g., substituted or unsubstituted C₃₋₆ heterocyclylene, substituted or unsubstituted C₃₋₄ heterocyclylene, substituted or unsubstituted C₄₋₅ heterocyclylene, or substituted or unsubstituted C₅₋₆ heterocyclylene.

In certain embodiments, L₁ is a group which comprises at least one instance of substituted or unsubstituted arylene, e.g., substituted or unsubstituted phenylene.

In certain embodiments, L₁ is a group which comprises at least one instance of substituted or unsubstituted heteroarylene, e.g., substituted or unsubstituted 5- to 6-membered heteroarylene.

In certain embodiments, L₂ is a bond.

In certain embodiments, L₂ is a group consisting of one or more combinations of substituted or unsubstituted alkylene; substituted or unsubstituted alkenylene; substituted or unsubstituted alkynylene; substituted or unsubstituted heteroalkylene; substituted or unsubstituted heteroalkenylene; substituted or unsubstituted heteroalkynylene; substituted or unsubstituted heterocyclene, substituted or unsubstituted carbocyclene, substituted or unsubstituted arylene; substituted or unsubstituted heteroarylene.

In certain embodiments, L₂ is a group which comprises at least one instance of substituted or unsubstituted alkylene, e.g., substituted or unsubstituted C₁₋₆alkylene, substituted or unsubstituted C₁₋₂alkylene, substituted or unsubstituted C₂₋₃alkylene, substituted or unsubstituted C₃₋₄alkylene, substituted or unsubstituted C₄₋₅alkylene, or substituted or unsubstituted C₅₋₆alkylene. Exemplary alkylene groups include unsubstituted alkylene groups such as methylene —CH₂—, ethylene —(CH₂)₂—, n-propylene —(CH₂)₃—, n-butylene —(CH₂)₄—, n-pentylene —(CH₂)₅—, and n-hexylene —(CH₂)₆—. In certain embodiments, L₂ is —CH₂—. In certain embodiments, L₂ is —(CH₂)₂—. In certain embodiments, L₂ is —(CH₂)₃—. In certain embodiments, L₂ is —(CH₂)₄—. In certain embodiments, L₂ is —(CH₂)₅—. In certain embodiments, L₂ is —(CH₂)₆—.

In certain embodiments, L₂ is a group which comprises at least one instance of substituted or unsubstituted alkenylene, e.g., substituted or unsubstituted C₂₋₄alkenylene, substituted or unsubstituted C₂₋₃alkenylene, substituted or unsubstituted C₃₋₄alkenylene, substituted or unsubstituted C₄₋₅alkenylene, or substituted or unsubstituted C₅₋₆alkenylene.

In certain embodiments, L₂ is a group which comprises at least one instance of substituted or unsubstituted alkynylene, e.g., substituted or unsubstituted C₂₋₆alkynylene, substituted or unsubstituted C₂₋₃alkynylene, substituted or unsubstituted C₃₋₄alkynylene, substituted or unsubstituted C₄₋₅alkynylene, or substituted or unsubstituted C₅₋₆alkynylene.

In certain embodiments, L₂ is a group which comprises at least one instance of substituted or unsubstituted heteroalkylene, e.g., substituted or unsubstituted heteroC₁₋₆alkylene, substituted or unsubstituted heteroC₁₋₂alkylene, substituted or unsubstituted heteroC₂₋₃alkylene, substituted or unsubstituted heteroC₃₋₄alkylene, substituted or unsubstituted heteroC₄₋₅alkylene, or substituted or unsubstituted heteroC₅₋₆alkylene. Exemplary heteroalkylene groups include unsubstituted alkylene groups such as —(CH₂)₂—O(CH₂)₂—, —OCH₂—, —O(CH₂)₂—, —O(CH₂)₃—, —O(CH₂)₄—, —O(CH₂)₅—, and —O(CH₂)₆—.

In certain embodiments, L₂ is a group which comprises at least one instance of substituted or unsubstituted heteroalkenylene, e.g., substituted or unsubstituted heteroC₂₋₆alkenylene, substituted or unsubstituted heteroC₂₋₃alkenylene, substituted or unsubstituted heteroC₃₋₄alkenylene, substituted or unsubstituted heteroC₄₋₅alkenylene, or substituted or unsubstituted heteroC₅₋₆alkenylene.

In certain embodiments, L₂ is a group which comprises at least one instance of substituted or unsubstituted heteroalkynylene, e.g., substituted or unsubstituted heteroC₂₋₆alkynylene, substituted or unsubstituted heteroC₂₋₃alkynylene, substituted or unsubstituted heteroC₃₋₄alkynylene, substituted or unsubstituted heteroC₄₋₅alkynylene, or substituted or unsubstituted heteroC₅₋₆alkynylene.

In certain embodiments, L₂ is a group which comprises at least one instance of substituted or unsubstituted carbocyclylene, e.g., substituted or unsubstituted C₃₋₆carbocyclylene, substituted or unsubstituted C₃₋₄carbocyclylene, substituted or unsubstituted C₄₋₅ carbocyclylene, or substituted or unsubstituted C₅₋₆ carbocyclylene.

In certain embodiments, L₂ is a group which comprises at least one instance of substituted or unsubstituted heterocyclylene, e.g., substituted or unsubstituted C₃₋₆ heterocyclylene, substituted or unsubstituted C₃₋₄ heterocyclylene, substituted or unsubstituted C₄₋₅ heterocyclylene, or substituted or unsubstituted C₅₋₆ heterocyclylene.

In certain embodiments, L₂ is a group which comprises at least one instance of substituted or unsubstituted arylene, e.g., substituted or unsubstituted phenylene.

In certain embodiments, L₂ is a group which comprises at least one instance of substituted or unsubstituted heteroarylene, e.g., substituted or unsubstituted 5- to 6-membered heteroarylene.

In certain embodiments, M is a bond.

In certain embodiments, M is a group consisting of one or more combinations of substituted or unsubstituted alkylene; substituted or unsubstituted alkenylene; substituted or unsubstituted alkynylene; substituted or unsubstituted heteroalkylene; substituted or unsubstituted heteroalkenylene; substituted or unsubstituted heteroalkynylene; substituted or unsubstituted heterocyclene, substituted or unsubstituted carbocyclene, substituted or unsubstituted arylene; substituted or unsubstituted heteroarylene.

In certain embodiments, M is a group which comprises at least one instance of substituted or unsubstituted alkylene, e.g., substituted or unsubstituted C₁₋₆alkylene, substituted or unsubstituted C₁₋₂alkylene, substituted or unsubstituted C₂₋₃alkylene, substituted or unsubstituted C₃₋₄alkylene, substituted or unsubstituted C₄₋₅alkylene, or substituted or unsubstituted C₅₋₆alkylene. Exemplary alkylene groups include unsubstituted alkylene groups such as methylene —CH₂—, ethylene —(CH₂)₂—, n-propylene —(CH₂)₃—, n-butylene —(CH₂)₄—, n-pentylene —(CH₂)₅—, and n-hexylene —(CH₂)₆—. In certain embodiments, M is —CH₂—. In certain embodiments, M is —(CH₂)₂—. In certain embodiments, M is —(CH₂)₃—. In certain embodiments, M is —(CH₂)₄—. In certain embodiments, M is —(CH₂)₅—. In certain embodiments, M is —(CH₂)₆—.

In certain embodiments, M is a group which comprises at least one instance of substituted or unsubstituted alkenylene, e.g., substituted or unsubstituted C₂₋₄alkenylene, substituted or unsubstituted C₂₋₃alkenylene, substituted or unsubstituted C₃₋₄alkenylene, substituted or unsubstituted C₄₋₅alkenylene, or substituted or unsubstituted C₅₋₆alkenylene.

In certain embodiments, M is a group which comprises at least one instance of substituted or unsubstituted alkynylene, e.g., substituted or unsubstituted C₂₋₆alkynylene, substituted or unsubstituted C₂₋₃alkynylene, substituted or unsubstituted C₃₋₄alkynylene, substituted or unsubstituted C₄₋₅alkynylene, or substituted or unsubstituted C₅₋₆alkynylene.

In certain embodiments, M is a group which comprises at least one instance of substituted or unsubstituted heteroalkylene, e.g., substituted or unsubstituted heteroC₁₋₆alkylene, substituted or unsubstituted heteroC₁₋₂alkylene, substituted or unsubstituted heteroC₂₋₃alkylene, substituted or unsubstituted heteroC₃₋₄alkylene, substituted or unsubstituted heteroC₄₋₅alkylene, or substituted or unsubstituted heteroC₅₋₄alkylene. Exemplary heteroalkylene groups include unsubstituted alkylene groups such as —(CH₂)₂—O(CH₂)₂—, —OCH₂—, —O(CH₂)₂—, —O(CH₂)₃—, —O(CH₂)₄—, —O(CH₂)₅—, and —O(CH₂)₆—.

In certain embodiments, M is a group which comprises at least one instance of substituted or unsubstituted heteroalkenylene, e.g., substituted or unsubstituted heteroC₂₋₆alkenylene, substituted or unsubstituted heteroC₂₋₃alkenylene, substituted or unsubstituted heteroC₃₋₄alkenylene, substituted or unsubstituted heteroC₄₋₅alkenylene, or substituted or unsubstituted heteroC₅₋₆alkenylene.

In certain embodiments, M is a group which comprises at least one instance of substituted or unsubstituted heteroalkynylene, e.g., substituted or unsubstituted heteroC₂₋₆alkynylene, substituted or unsubstituted heteroC₂₋₃alkynylene, substituted or unsubstituted heteroC₃₋₄alkynylene, substituted or unsubstituted heteroC₄₋₅alkynylene, or substituted or unsubstituted heteroC₅₋₆alkynylene.

In certain embodiments, M is a group which comprises at least one instance of substituted or unsubstituted carbocyclylene, e.g., substituted or unsubstituted C₃₋₆carbocyclylene, substituted or unsubstituted C₃₋₄carbocyclylene, substituted or unsubstituted C₄₋₅ carbocyclylene, or substituted or unsubstituted C₅₋₆ carbocyclylene.

In certain embodiments, M is a group which comprises at least one instance of substituted or unsubstituted heterocyclylene, e.g., substituted or unsubstituted C₃₋₆ heterocyclylene, substituted or unsubstituted C₃₋₄ heterocyclylene, substituted or unsubstituted C₄₋₅ heterocyclylene, or substituted or unsubstituted C₅₋₆ heterocyclylene.

In certain embodiments, M is a group which comprises at least one instance of substituted or unsubstituted arylene, e.g., substituted or unsubstituted phenylene.

In certain embodiments, M is a group which comprises at least one instance of substituted or unsubstituted heteroarylene, e.g., substituted or unsubstituted 5- to 6-membered heteroarylene.

Groups R^{a} and R^{b}

As generally defined above, R^{a} is hydrogen; substituted or unsubstituted aliphatic; substituted or unsubstituted heteroaliphatic; substituted or unsubstituted aryl; substituted or unsubstituted heteroaryl; substituted or unsubstituted acyl; a resin; an amino protecting group; a label optionally joined by a linker, wherein the linker is a group consisting of one or more combinations of substituted or unsubstituted alkylene; substituted or unsubstituted alkenylene; substituted or unsubstituted alkynylene; substituted or unsubstituted heteroalkylene; substituted or unsubstituted heteroalkenylene; substituted or unsubstituted heteroalkynylene; substituted or unsubstituted carbocyclene; substituted or unsubstituted heterocyclene; substituted or unsubstituted arylene; or substituted or unsubstituted heteroarylene.

In certain embodiments, R^{a} is hydrogen.

In certain embodiments, R^{a} is substituted or unsubstituted aliphatic; i.e., substituted or unsubstituted alkyl, alkenyl, alkynyl, or carbocyclyl.

In certain embodiments, R^{a} is substituted or unsubstituted alkyl, e.g., substituted or unsubstituted C₁₋₆alkyl, substituted or unsubstituted C₁₋₂alkyl, substituted or unsubstituted C₂₋₃alkyl, substituted or unsubstituted C₃₋₄alkyl, substituted or unsubstituted C₄₋₅alkyl, or substituted or unsubstituted C₅₋₅alkyl. Exemplary R^{a}C₁₋₆alkyl groups include, but are not limited to, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆).

In certain embodiments, R^{a} is substituted or unsubstituted heteroaliphatic; i.e., substituted or unsubstituted heteroalkyl, heteroalkenyl, heteroalkynyl, or heterocyclyl.

In certain embodiments, R^{a} is substituted or unsubstituted aryl;

In certain embodiments, R^{a} is substituted or unsubstituted heteroaryl.

In certain embodiments, R^{a} is substituted or unsubstituted acyl, e.g., acetyl —C(═O)CH₃.

In certain embodiments, R^{a} is a resin.

In certain embodiments, R^{a} is an amino protecting group.

In certain embodiments, R^{a} is a label optionally joined by a linker.

### Group R^{b}

As generally defined above, R^{b} is, —R^{B}, —OR^{B}, —N(R^{B})₂, or —SR^{B}, wherein each instance of R^{B} is, independently, hydrogen, substituted or unsubstituted aliphatic; substituted or unsubstituted heteroaliphatic; substituted or unsubstituted aryl; substituted or unsubstituted heteroaryl; substituted or unsubstituted acyl; a resin; a suitable hydroxyl, amino or thiol protecting group; or two R^{B} groups together form a substituted or unsubstituted 5- to 6-membered heterocyclic or heteroaromatic ring.

In certain embodiments, R^{b} is —R^{B}, e.g., R^{b} is hydrogen, substituted or unsubstituted aliphatic; substituted or unsubstituted heteroaliphatic; substituted or unsubstituted aryl; substituted or unsubstituted heteroaryl. In certain embodiments, R^{b} is substituted or unsubstituted aliphatic, e.g., substituted or unsubstituted alkyl, alkenyl, alkynyl, or carbocyclyl.

In certain embodiments, R^{b} is —OR^{B}, e.g., —OH.

In certain embodiments, R^{b} is —N(R^{B})₂, e.g., —NH(C═O)CH₃.

In certain embodiments, R^{b} is —SR^{B}, e.g., —SH.

### Group Rc and Variable x

As generally defined above, each instance of R^{c}, is, independently, hydrogen; substituted or unsubstituted aliphatic; substituted or unsubstituted heteroaliphatic; substituted or unsubstituted aryl; substituted or unsubstituted heteroaryl; substituted or unsubstituted acyl; substituted or unsubstituted hydroxyl; substituted or unsubstituted thiol; substituted or unsubstituted amino; cyano; isocyano; halo; or nitro, and each instance of x is, independently, 0, 1, 2, or 3.

In certain embodiments, each instance of x is 0 and R^{c} is thus absent. In certain embodiments at least one instance of x is 1, and thus at least one instance of R^{c} is a non-hydrogen substituent.

Groups R^{KL}, R^{LL}, and R^{LM} and Variable q

As generally defined above, each instance of R^{KL}, R^{LL}, and R^{LM}, is, independently, hydrogen; substituted or unsubstituted aliphatic; substituted or unsubstituted heteroaliphatic; substituted or unsubstituted aryl; substituted or unsubstituted heteroaryl; substituted or unsubstituted acyl; substituted or unsubstituted hydroxyl; substituted or unsubstituted thiol, substituted or unsubstituted amino; azido; cyano; isocyano; halo; nitro; and each instance of q is, independently 0, 1, or 2.

In certain embodiments, each instance of q is 0 and R^{KL}, R^{LL}, and R^{LM}, are thus absent. In certain embodiments at least one instance of q is 1, and thus at least one instance of R^{KL}, R^{LL}, and R^{LM}, is anon-hydrogen substituent.

### Group R^{d}

As generally defined above, each instance of R^{d} is, independently, hydrogen; substituted or unsubstituted aliphatic; substituted or unsubstituted heteroaliphatic; substituted or unsubstituted aryl; substituted or unsubstituted heteroaryl; substituted or unsubstituted acyl; or an amino protecting group.

In certain embodiments, each instance of R^{d} is hydrogen or substituted or unsubstituted aliphatic, e.g., substituted or unsubstituted alkyl, alkenyl, alkynyl, or carbocyclyl. In certain embodiments, each instance of R^{d} is hydrogen or substituted or unsubstituted alkyl, e.g., —CH₃.

### Group R^{e}

As generally defined above, each instance of R^{e} is, independently, hydrogen; substituted or unsubstituted aliphatic; substituted or unsubstituted heteroaliphatic; substituted or unsubstituted aryl; substituted or unsubstituted heteroaryl; substituted or unsubstituted acyl; substituted or unsubstituted hydroxyl; substituted or unsubstituted thiol; substituted or unsubstituted amino; cyano; isocyano; halo; or nitro.

In certain embodiments, each instance of R^{e} is hydrogen or substituted or unsubstituted aliphatic, e.g., substituted or unsubstituted alkyl, alkenyl, alkynyl, or carbocyclyl. In certain embodiments, each instance of R^{e} is hydrogen or substituted or unsubstituted, e.g., —CH₃, —CH₂OH, —COOH, or —CH₂COOH. In certain embodiments, R^{e} is —CH₃.

### Group G and Variable v

As generally defined above, each instance of G is, independently, a natural or unnatural amino acid or a group of the formula:

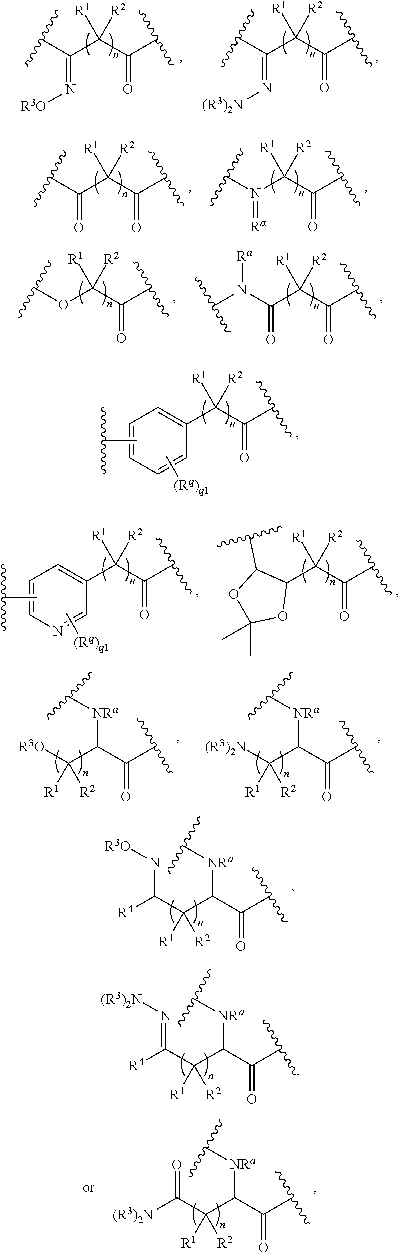

wherein:
n is 1, 2, or 3; and
each instance of R¹ and R² is independently hydrogen; substituted or unsubstituted aliphatic; substituted or unsubstituted heteroaliphatic; substituted or unsubstituted aryl; substituted or unsubstituted heteroaryl; substituted or unsubstituted acyl; substituted or unsubstituted hydroxyl; substituted or unsubstituted thiol; substituted or unsubstituted amino; or halo, or R¹ and R² are joined to form a carbocyclic or heterocyclic ring;
each instance of R³ and R⁴ is, independently, hydrogen; substituted or unsubstituted aliphatic; substituted or unsubstituted heteroaliphatic; a hydroxyl protecting group when attached to an oxygen atom, or an amino protecting group when attached to a nitrogen atom, or two R³ groups when attached to a nitrogen atom are joined to form a heterocyclic ring;
each instance of R^{q} is independently halogen, —CN, —NO₂, —N₃, optionally substituted alkyl, optionally substituted alkenyl, optionally substituted alkynyl, optionally substituted carbocyclyl, optionally substituted phenyl, optionally substituted heterocyclyl, optionally substituted heteroaryl, optionally substituted alkoxy, an optionally substituted amino group, or optionally substituted acyl;
q1 is 0, 1, 2, 3, or 4; and
each instance of v is, independently, 0 or 1.

In certain embodiments, v is 0 and G in that particular instance is absent.

However, in certain embodiments, v is 1.

In certain embodiments, G is independently, serine, arginine, aspartic acid, or glutamic acid. In certain embodiments, G is serine. In certain embodiments, G is arginine. In certain embodiments, G is aspartic acid. In certain embodiments, G is glutamic acid.

In certain embodiments, G is a group of formula:

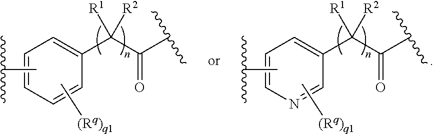

In certain embodiments, G is a group of formula:

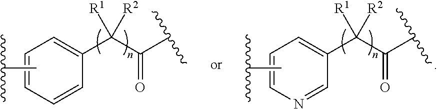

In certain embodiments, G is a group of formula:

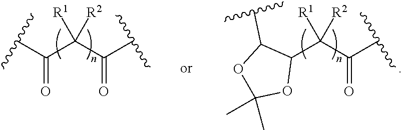

In certain embodiments, G is a group of formula:

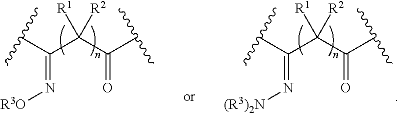

In certain embodiments, G is a group of formula:

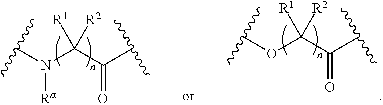

In certain embodiments, G is a group of formula:

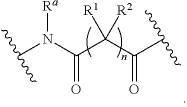

In certain embodiments, G is a group of formula:

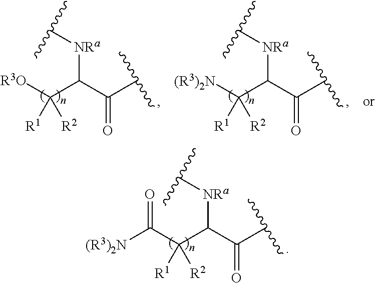

In certain embodiments, G is a group of formula:

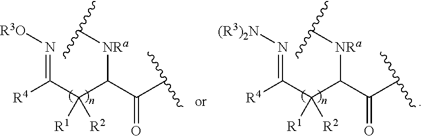

In certain embodiments, n is 1. In certain embodiments, n is 2. In certain embodiments, n is 3.

In certain embodiments, at least one instance of R¹ is hydrogen.

In certain embodiments, at least one instance of R¹ is substituted or unsubstituted aliphatic, e.g., substituted or unsubstituted alkyl, alkenyl, alkynyl, or carbocyclyl. In certain embodiments, at least one instance of R¹ is substituted or unsubstituted alkyl, e.g., —CH₃.

In certain embodiments, at least one instance of R² is hydrogen.

In certain embodiments, at least one instance of R² is substituted or unsubstituted aliphatic, e.g., substituted or unsubstituted alkyl, alkenyl, alkynyl, or carbocyclyl. In certain embodiments, at least one instance of R² is substituted or unsubstituted alkyl, e.g., —CH₃.

In certain embodiments, at least one instance of R¹ is hydrogen and at least one instance of R² is hydrogen.

In certain embodiments, at least one instance of R¹ is hydrogen and at least one instance of R² is substituted or unsubstituted alkyl, e.g., —CH₃.

In certain embodiments, at least one instance of R¹ and R² is substituted or unsubstituted alkyl, e.g., substituted or unsubstituted C₁₋₆alkyl, substituted or unsubstituted C₁₋₂alkyl, substituted or unsubstituted C₂₋₃alkyl, substituted or unsubstituted C₃₋₄alkyl, substituted or unsubstituted C₄₋₅alkyl, or substituted or unsubstituted C₅₋₆alkyl. Exemplary C₁₋₆alkyl groups include, but are not limited to, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆).

In certain embodiments, at least one instance of R³ is hydrogen or substituted or unsubstituted aliphatic, e.g., substituted or unsubstituted alkyl, alkenyl, alkynyl, or carbocyclyl. In certain embodiments, at least one instance of R³ is substituted or unsubstituted alkyl, e.g., substituted or unsubstituted C₁₋₆alkyl, substituted or unsubstituted C₁₋₂alkyl, substituted or unsubstituted C₂₋₃alkyl, substituted or unsubstituted C₃₋₄alkyl, substituted or unsubstituted C₄₋₅alkyl, or substituted or unsubstituted C₅₋₆alkyl. Exemplary C₁₋₆alkyl groups include, but are not limited to, substituted or unsubstituted methyl (C₁), ethyl (C₂), n-propyl (C₃), isopropyl (C₃), n-butyl (C₄, tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆).

In certain embodiments, two instance of R³ when attached to the same nitrogen atom are joined to form a heterocyclic ring.

In certain embodiments, R⁴ is hydrogen or substituted or unsubstituted aliphatic, e.g., substituted or unsubstituted alkyl, alkenyl, alkynyl, or carbocyclyl. In certain embodiments, R⁴ is substituted or unsubstituted alkyl, e.g., substituted or unsubstituted C₁₋₆alkyl, substituted or unsubstituted C₁₋₂alkyl, substituted or unsubstituted C₂₋₃alkyl, substituted or unsubstituted C₃₋₄alkyl, substituted or unsubstituted C₄₋₅alkyl, or substituted or unsubstituted C₅₋₆alkyl. Exemplary C₁₋₆alkyl groups include, but are not limited to, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆).

In certain embodiments, q1 is 0. In certain embodiments, q1 is 1. In certain embodiments, q1 is 2. In certain embodiments, q1 is 3. In certain embodiments, q1 is 4.

In certain embodiments, R^{q} is halogen, —CN, —NO₂, —N₃, or optionally substituted alkyl.

In certain embodiments,

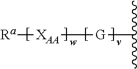

in Formula (P-I), (I), (P-II), and (II) is one of the following formulae:

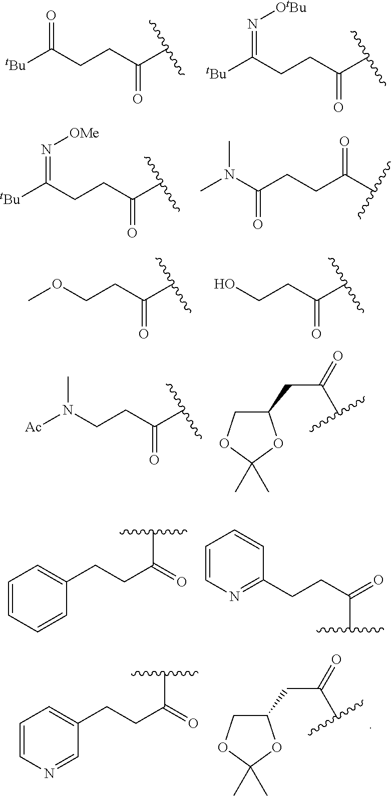

Group XAA and variables j, y, p, w, z, s and t

As generally defined above, each instance of X_{AA} is, independently, a natural or unnatural amino acid. Various natural and unnatural amino acids are generally described herein, and encompass alpha and beta amino acids moieties joined via peptide bonds.

As generally defined above, and each instance of t, w and z is, independently, an integer between 0 and 100, inclusive.

In certain embodiments z is an integer of 1 to 10, inclusive. In certain embodiments z is an integer of 2 to 10, inclusive. In certain embodiments, z is 1. In certain embodiments, z is 2. In certain embodiments, z is 3. In certain embodiments, z is 4. In certain embodiments, z is 5. In certain embodiments, z is 6. In certain embodiments, z is 7. In certain embodiments, z is 9. In certain embodiments, z is 10.

In certain embodiments w is 0, 1, or 2 and z is an integer between 0 and 100, inclusive. In certain embodiments w is 0, 1, or 2 and z is an integer between 0 and 75, inclusive. In certain embodiments w is 0, 1, or 2 and z is an integer between 0 and 50, inclusive. In certain embodiments w is 0, 1, or 2 and z is an integer between 0 and 25, inclusive. In certain embodiments w is 0, 1, or 2 and z is an integer between 0 and 10, inclusive. In certain embodiments w is 0, 1, or 2 and z is an integer between 0 and 5, inclusive.

In certain embodiments w is 0.

In certain embodiments w is 0, 1, or 2 and t is an integer between 0 and 100, inclusive. In certain embodiments w is 0, 1, or 2 and t is an integer between 0 and 75, inclusive. In certain embodiments w is 0, 1, or 2 and t is an integer between 0 and 50, inclusive. In certain embodiments w is 0, 1, or 2 and t is an integer between 0 and 25, inclusive. In certain embodiments w is 0, 1, or 2 and t is an integer between 0 and 10, inclusive. In certain embodiments w is 0, 1, or 2 and t is an integer between 0 and 5, inclusive.

As generally defined above, y is independently, an integer of 1 to 8, inclusive. In certain embodiments, y is independently, an integer of 1 to 7, inclusive. In certain embodiments, y is independently, an integer of 1 to 6, inclusive. In certain embodiments, y is independently, an integer of 1 to 5, inclusive. In certain embodiments, y is independently, 1, 2, 3, or 4. In certain embodiments, y is 1. In certain embodiments, y is 2. In certain embodiments, y is 3. In certain embodiments, y is 4. In certain embodiments, y is 5. In certain embodiments, y is 6. In certain embodiments, y is 7. In certain embodiments, y is 8.

As generally defined above, j is, independently, an integer between 1 to 10, inclusive, e.g., j is 1, 2, 3, 4, 5, 6, 7, 8, 9, or 10. In certain embodiments, j is 1. In certain embodiments, j is 2.

As generally defined above, each instance of p is, independently, 1 or 2. In certain embodiments, p is 1. In certain embodiments, p is 2.

In certain embodiments, j is 1 and p is 1. In certain embodiments, j is 1 and p is 2.

In certain embodiments, j is 1, p is 1, and y is 1. In certain embodiments, j is 1, p is 1, and y is 2. In certain embodiments, j is 1, p is 1, and y is 3. In certain embodiments, j is 1, p is 1, and y is 4.

In certain embodiments, j is 1, p is 2, and y is 1. In certain embodiments, j is 1, p is 2, and y is 2. In certain embodiments, j is 1, p is 2, and y is 3. In certain embodiments, j is 1, p is 2, and y is 4.

As generally defined above, s is 0, 1, or 2. In certain embodiments, s is 0. In certain embodiments, s is 1. In certain embodiments, s is 2.

In certain embodiments, j is 1, p is 1, and s is 0 or 1. In certain embodiments, j is 1, p is 2, and s is 0 or 1.

In certain embodiments, j is 1, p is 1, s is 0 or 1, and y is 1. In certain embodiments, j is 1, p is 1, s is 0 or 1, and y is 2. In certain embodiments, j is 1, p is 1, s is 0 or 1, and y is 3. In certain embodiments, j is 1, p is 1, s is 0 or 1, and y is 4.

In certain embodiments, j is 1, p is 2, s is 0 or 1, and y is 1. In certain embodiments, j is 1, p is 2, s is 0 or 1, and y is 2. In certain embodiments, j is 1, p is 2, s is 0 or 1, and y is 3. In certain embodiments, j is 1, p is 2, s is 0 or 1, and y is 4.

The variables y and z indicate how many amino acids, defined by the variable [X_{AA}], there are between amino acids containing terminally unsaturated amino acid side chain(s), as provided in polypeptides of Formulae (P-I), (P-II), (I) and (II). For example, as depicted below for a polypeptide of Formula (P-II), wherein s is 0; i represents one site of an alpha,alpha-disubstituted (terminally unsaturated amino acid side chain) amino acid, variable y provides information as to the position of the amino acid containing a terminally unsaturated side chain on the N-terminal side of i, such as the positions i−3, i−4, i−6, and i−7, and z provides information as to the position of the amino acid containing a terminally unsaturated side chain on the C-terminal side of i, such as the positions i+3, i+4, i+6, and i+7. Table 4 correlates these specific locations of i relative to the variables y and z for formula (P-II-a).

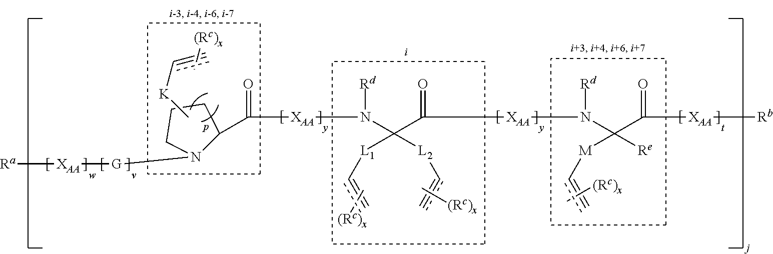

TABLE 4

| | i − 7 | i − 6 | i − 4 | i − 3 | i | i + 3 | i + 4 | i + 6 | i + 7 |
|---|---|---|---|---|---|---|---|---|---|
| y | 6 | 5 | 3 | 2 | | | | | |
| z | | | | | | 2 | 3 | 5 | 6 |

In certain embodiments, each instance of y and z are, independently, 2, 3, 5, or 6.

In certain embodiments, both y and z are 2. In certain embodiments, both y and z are 3. In certain embodiments, both y and z are 5. In certain embodiments, both y and z are 6.

In certain embodiments, y is 2 and z is 3. In certain embodiments, y is 2 and z is 5. In certain embodiments, y is 2 and z is 6.

In certain embodiments, y is 3 and z is 2. In certain embodiments, y is 3 and z is 5. In certain embodiments, y is 3 and z is 6.

In certain embodiments, y is 5 and z is 2. In certain embodiments, y is 5 and z is 3. In certain embodiments, y is 5 and z is 6.

In certain embodiments, y is 6 and z is 2. In certain embodiments, y is 6 and z is 3. In certain embodiments, y is 6 and z is 5.

### Additional Embodiments

Various combinations of the above embodiments are contemplated herein.

For example, in certain embodiments of Formula (P-I) and (I), K and L are —CH₂—; R^{d} is —H; R^{e} is —CH₂OH; j is 1; p is 1; v is 0; x is 0; y is 2; and, [symbol] corresponds to a double bond.

For example, in certain embodiments of Formula (P-I) and (I), K and L are independently —CH₂CH₂— or —OCH₂—; R^{d} is —H; R^{e} is —CH₂OH; j is 1; p is 1; v is 0; x is 0; y is 2; and, [symbol] corresponds to a double bond.

For example, in certain embodiments of Formula (P-I) and (I), K and L are —CH₂CH₂CH₂—; R^{d} is —H; R^{e} is —CH₂OH; j is 1; p is 1; v is 0; x is 0; y is 2; and, [symbol] corresponds to a double bond.

For example, in certain embodiments of Formula (P-I) and (I), K and L are —CH₂—; R^{d} is —H; R^{e} is —CH₂CH₂OH; j is 1; p is 1; v is 0; x is 0; y is 2; and, [symbol] corresponds to a double bond.

For example, in certain embodiments of Formula (P-I) and (I), K and L are —CH₂—; R^{d} is —H; R^{e} is —H; j is 1; p is 1; v is 0; x is 0; y is 2; and, [symbol] corresponds to a double bond.

For example, in certain embodiments of Formula (P-I) and (I), K and L are —CH₂—; R^{d} is —H; R^{e} is —CH₃; j is 1; p is 1; v is 0; x is 0; y is 2; and, [symbol] corresponds to a double bond. In certain embodiments of Formula (P-I) and (I), K is —CH₂—; L is —(CH₂)₂—; R^{d} is —H; R^{e} is —CH₃; j is 1; p is 1; v is 0; x is 0; y is 2; and, [symbol] corresponds to a double bond. In certain embodiments of Formula (P-I) and (I), K is —CH₂—; L is —(CH₂)₃—; R^{d} is —H; R^{e} is —CH₃; j is 1; p is 1; v is 0; x is 0; y is 2; and, [symbol] corresponds to a double bond.

For example, in certain embodiments of Formula (P-I) and (I), K and L are —CH₂—; R^{d} is —H; R^{e} is —CH₃; j is 1; p is 1; v is 0; x is 0; y is 3; and, [symbol] corresponds to a double bond. In certain embodiments of Formula (P-I) and (I), K is —CH₂—; L is —(CH₂)₂—; R^{d} is —H; R^{e} is —CH₃; j is 1; p is 1; v is 0; x is 0; y is 3; and, [symbol] corresponds to a double bond. In certain embodiments of Formula (P-I) and (I), K is —CH₂—; L is —(CH₂)₃—; R^{d} is —H; R^{e} is —CH₃; j is 1; p is 1; v is 0; x is 0; y is 3; and, [symbol] corresponds to a double bond.

For example, in certain embodiments of Formula (P-I) and (I), K and L are —CH₂—; R^{d} is —H; R^{e} is —CH₃; j is 1; p is 1; v is 0; x is 0; y is 5; and, [symbol] corresponds to a double bond. In certain embodiments of Formula (P-I) and (I), K is —CH₂—; L is —(CH₂)₂—; R^{d} is —H; R^{e} is —CH₃; j is 1; p is 1; v is 0; x is 0; y is 5; and, [symbol] corresponds to a double bond. In certain embodiments of Formula (P-I) and (I), K is —CH₂—; L is —(CH₂)₃—; R^{d} is —H; R^{e} is —CH₃; j is 1; p is 1; v is 0; x is 0; y is 5; and, [symbol] corresponds to a double bond.

For example, in certain embodiments of Formula (P-I) and (I), K and L are —CH₂—; R^{d} is —H; R^{e} is —CH₃; j is 1; p is 1; v is 0; x is 0; y is 6; and, [symbol] corresponds to a double bond. In certain embodiments of Formula (P-I) and (I), K is —CH₂—; L is —(CH₂)₂—; R^{d} is —H; R^{e} is —CH₃; j is 1; p is 1; v is 0; x is 0; y is 6; and, [symbol] corresponds to a double bond. In certain embodiments of Formula (P-I) and (I), K is —CH₂—; L is —(CH₂)₃—; R^{d} is —H; R^{e} is —CH₃; j is 1; p is 1; v is 0; x is 0; y is 6; and, [symbol] corresponds to a double bond.

For example, in certain embodiments of Formula (P-I) and (I), K is —CH₂O—, wherein O is linked to the heterocyclyl with nitrogen and CH₂ is linked to “ [symbol] ,” and L is —CH₂—; R^{d} is —H; R^{e} is —CH₃; j is 1; p is 1; v is 0; x is 0; y is 2; and, [symbol] corresponds to a double bond. In certain embodiments of Formula (P-I) and (I), K is —CH₂O—, wherein O is linked to the heterocyclyl with nitrogen and CH₂ is linked to “ [symbol] ,” and L is —(CH₂)₂—; R^{d} is —H; R^{e} is —CH₃; j is 1; p is 1; v is 0; x is 0; y is 2; and, [symbol] corresponds to a double bond. In certain embodiments of Formula (P-I) and (I), K is —CH₂O—, wherein O is linked to the heterocyclyl with nitrogen and CH₂ is linked to “ [symbol] ,” and L is —(CH₂)₃—; R^{d} is —H; R^{e} is —CH₃; j is 1; p is 1; v is 0; x is 0; y is 2; and, [symbol] corresponds to a double bond.

For example, in certain embodiments of Formula (P-I) and (I), K is —CH₂O—, wherein O is linked to the heterocyclyl with nitrogen and CH₂ is linked to “ [symbol] ,” and L is —CH₂—; R^{d} is —H; R^{e} is —CH₃; j is 1; p is 1; v is 0; x is 0; y is 3; and, [symbol] corresponds to a double bond. In certain embodiments of Formula (P-I) and (I), K is —CH₂O—, wherein O is linked to the heterocyclyl with nitrogen and CH₂ is linked to “ [symbol] ,” and L is —(CH₂)₂—; R^{d} is —H; R^{e} is —CH₃; j is 1; p is 1; v is 0; x is 0; y is 3; and, [symbol] corresponds to a double bond. In certain embodiments of Formula (P-I) and (I), K is —CH₂O—, wherein O is linked to the heterocyclyl with nitrogen and CH₂ is linked to “ [symbol] ,” and L is —(CH₂)₃—; R^{d} is —H; R^{e} is —CH₃; j is 1; p is 1; v is 0; x is 0; y is 3; and, [symbol] corresponds to a double bond.

For example, in certain embodiments of Formula (P-I) and (I), K is —CH₂O—, wherein O is linked to the heterocyclyl with nitrogen and CH₂ is linked to “ [symbol] ,” and L is —CH₂—; R^{d} is —H; R^{e} is —CH₃; j is 1; p is 1; v is 0; x is 0; y is 5; and, [symbol] corresponds to a double bond. In certain embodiments of Formula (P-I) and (I), K is —CH₂O—, wherein 0 is linked to the heterocyclyl with nitrogen and CH₂ is linked to “ [symbol] ,” and L is —(CH₂)₂—; R^{d} is —H; R^{e} is —CH₃; j is 1; p is 1; v is 0; x is 0; y is 5; and, [symbol] corresponds to a double bond. In certain embodiments of Formula (P-I) and (I), K is —CH₂O—, wherein 0 is linked to the heterocyclyl with nitrogen and CH₂ is linked to “ [symbol] ,” and L is —(CH₂)₃—; R^{d} is —H; R^{e} is —CH₃; j is 1; p is 1; v is 0; x is 0; y is 5; and, [symbol] corresponds to a double bond.

For example, in certain embodiments of Formula (P-I) and (I), K is —CH₂O—, wherein 0 is linked to the heterocyclyl with nitrogen and CH₂ is linked to “ [symbol] ,” and L is —CH₂—; R^{d} is —H; R^{e} is —CH₃; j is 1; p is 1; v is 0; x is 0; y is 6; and, [symbol] corresponds to a double bond. In certain embodiments of Formula (P-I) and (I), K is —CH₂O—, wherein 0 is linked to the heterocyclyl with nitrogen and CH₂ is linked to “ [symbol] ,” and L is —(CH₂)₂—; R^{d} is —H; R^{e} is —CH₃; j is 1; p is 1; v is 0; x is 0; y is 6; and, [symbol] corresponds to a double bond. In certain embodiments of Formula (P-I) and (I), K is —CH₂O—, wherein 0 is linked to the heterocyclyl with nitrogen and CH₂ is linked to “ [symbol] ,” and L is —(CH₂)₃—; R^{d} is —H; R^{e} is —CH₃; j is 1; p is 1; v is 0; x is 0; y is 6; and, [symbol] corresponds to a double bond.

For example, in certain embodiments of Formula (P-I) and (I), K and L are —CH₂—; R^{d} is —H; R^{e} is —CH₃COOH; j is 1; p is 1; v is 0; x is 0; y is 2; and, [symbol] corresponds to a double bond.

For example, in certain embodiments of Formula (P-I) and (I), K and L are —CH₂—; R^{d} is —H; R^{e} is —CH₂CH₂COOH; j is 1; p is 1; v is 0; x is 0; y is 2; and, [symbol] corresponds to a double bond.

For example, in certain embodiments of Formula (P-I) and (I), K and L are —CH₂—; R^{d} is —H; RV is —CH₂OH; j is 1; p is 2; x is 0; v is 0; y is 2; and, [symbol] corresponds to a double bond.

For example, in certain embodiments of Formula (P-I) and (I), K and L are —CH₂—; R^{d} is —H; R^{e} is —CH₂OH; j is 1; p is 1; v is 0; x is 2; y is 2; and, [symbol] corresponds to a double bond.

For example, in certain embodiments of Formula (P-I) and (I), K and L are —CH₂—; R^{d} is —H; R^{e} is —CH₂OH; j is 1; p is 1; v is 1; x is 0; y is 2; [G] is serine; and, [symbol] corresponds to a double bond.

For example, in certain embodiments of Formula (P-I) and (I), K and L are —CH₂—, R^{d} is —H; R^{e} is —CH₂OH; j is 1; p is 1; v is 1; x is 0; y is 2; [G] is threonine; and, [symbol] corresponds to a double bond.

For example, in certain embodiments of Formula (P-I) and (I), K and L are —CH₂—; R^{d} is —H; R^{e} is —CH₂OH; j is 1; p is 1; v is 1; x is 0; y is 2; [G] is aspartic acid; and, [symbol] corresponds to a double bond.

For example, in certain embodiments of Formula (P-I) and (I), K and L are —CH₂—; R^{d} is —H; R^{e} is —CH₂OH; j is 1; p is 1; v is 1; x is 0; y is 2; [G] is glutamic acid; and, [symbol] corresponds to a double bond.

For example, in certain embodiments of Formula (P-I) and (I), K and L are —CH₂—; R^{d} is —H; R^{e} is —CH₂OH; j is 1; p is 1; v is 1; x is 0; y is 2; [G] is asparagine; and, [symbol] corresponds to a double bond.

For example, in certain embodiments of Formula (P-I) and (I), K and L are —CH₂—; R^{d} is —H; R^{e} is —CH₂OH; j is 1; p is 1; v is 1; x is 0; y is 2; [G] is;

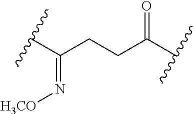 [SMILES: *C(=O)CC/C(*)=N/OC]

and, [symbol] corresponds to a double bond.

For example, in certain embodiments of Formula (P-I) and (I), K and L are —CH₂—, R^{d} is —H; R^{e} is —CH₂OH; j is 1; p is 1; q is 0; v is 0; y is 2; and, [symbol] corresponds to a double bond.

For example, in certain embodiments of Formula (P-I) and (I), K and L are —CH₂—; R^{d} is —H; R^{e} is —CH₂OH; j is 1; p is 1; q is 0; v is 1; y is 2; [G] is;

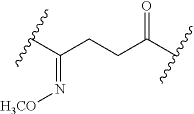 [SMILES: *C(=O)CC/C(*)=N/OC]

and, [symbol] corresponds to a double bond.

For example, in certain embodiments of Formula (P-II) and (II), L₁, L₂ and M are —CH₂—; R^{d} is —H; R^{e} is —H; j is 1; p is 1; s is 0; is 0; x is 0; y is 2; z is 2; and, [symbol] corresponds to a double bond. In certain embodiments of Formula (P-II) and (II), L₁, L₂ are —(CH₂)₃—; M is —CH₂—; R^{d} is —H; R^{e} is —CH₃; j is 1; p is 1; s is 0; v is 0; x is 0; y is 2; z is 2; and, [symbol] corresponds to a double bond. In certain embodiments of Formula (P-II) and (II), L₁, L₂ are —(CH₂)₃—; M is —(CH₂)₂—; R^{d} is —H; R^{e} is —CH₃; j is 1; p is 1; s is 0; v is 0; x is 0; y is 2; z is 2; and, [symbol] corresponds to a double bond. In certain embodiments of Formula (P-II) and (II), L₁, L₂ are —(CH₂)₃—; M is —(CH₂)₃—; R^{d} is —H; R^{e} is —CH₃; j is 1; p is 1; s is 0; v is 0; x is 0; y is 2; z is 2; and, [symbol] corresponds to a double bond. In certain embodiments of Formula (P-II) and (II), L₁, L₂ are —(CH₂)₃—; M is —(CH₂)₄—; R^{d} is —H; R^{e} is —CH₃; j is 1; p is 1; s is 0; v is 0; x is 0; y is 2; z is 2; and, [symbol] corresponds to a double bond. In certain embodiments of Formula (P-II) and (II), L₁, L₂ are —(CH₂)₃—; M is —(CH₂)₅—; R^{d} is —H; R^{e} is —CH₃; j is 1; p is 1; s is 0; v is 0; x is 0; y is 2; z is 2; and, [symbol] corresponds to a double bond. In certain embodiments of Formula (P-II) and (II), L₁, L₂ are —(CH₂)₃—; M is —(CH₂)₆—; R^{d} is —H; R^{e} is —CH₃; j is 1; p is 1; s is 0; v is 0; x is 0; y is 2; z is 2; and, [symbol] corresponds to a double bond.

For example, in certain embodiments of Formula (P-II) and (II), L₁, L₂ and M are —CH₂—; R^{d} is —H; R^{e} is —H; j is 1; p is 1; s is 0; v is 0; x is 0; y is 2; z is 3; and, [symbol] corresponds to a double bond. In certain embodiments of Formula (P-II) and (II), L₁, L₂ are —(CH₂)₃—; M is —CH₂—; R^{d} is —H; R^{e} is —CH₃; j is 1; p is 1; s is 0; v is 0; x is 0; y is 2; z is 3; and, [symbol] corresponds to a double bond. In certain embodiments of Formula (P-II) and (II), L₁, L₂ are —(CH₂)₃—; M is —(CH₂)₂—; R^{d} is —H; R^{e} is —CH₃; j is 1; p is 1; s is 0; v is 0; x is 0; y is 2; z is 3; and, [symbol] corresponds to a double bond. In certain embodiments of Formula (P-II) and (II), L₁, L₂ are —(CH₂)₃—; M is —(CH₂)₃—; R^{d} is —H; R^{e} is —CH₃; j is 1; p is 1; s is 0; v is 0; x is 0; y is 2; z is 3; and, [symbol] corresponds to a double bond. In certain embodiments of Formula (P-II) and (I), L₁, L₂ are —(CH₂)₃—; M is —(CH₂)₄—; R^{d} is —H; R^{e} is —CH₃; j is 1; p is 1; s is 0; v is 0; x is 0; y is 2; z is 3; and, [symbol] corresponds to a double bond. In certain embodiments of Formula (P-II) and (II), L₁, L₂ are —(CH₂)₃—; M is —(CH₂)₅—; R^{d} is —H; R^{e} is —CH₃; j is 1; p is 1; s is 0; v is 0; x is 0; y is 2; z is 3; and, [symbol] corresponds to a double bond. In certain embodiments of Formula (P-II) and (II), L₁, L₂ are —(CH₂)₃—; M is —(CH₂)₆—; R^{d} is —H; R^{e} is —CH₃; j is 1; p is 1; s is 0; v is 0; x is 0; y is 2; z is 3; and, [symbol] corresponds to a double bond.

For example, in certain embodiments of Formula (P-II) and (II), L₁, L₂ and M are —CH₂—; R^{d} is —H; R^{e} is —H; j is 1; p is 1; s is 0; v is 0; x is 0; y is 2; z is 6; and, [symbol] corresponds to a double bond. In certain embodiments of Formula (P-II) and (II), L₁, L₂ are —(CH₂)₃—; M is —CH₂—; R^{d} is —H; R^{e} is —CH₃; j is 1; p is 1; s is 0; v is 0; x is 0; y is 2; z is 6; and, [symbol] corresponds to a double bond. In certain embodiments of Formula (P-II) and (II), L₁, L₂ are —(CH₂)₃—; M is —(CH₂)₂—; R^{d} is —H; R^{e} is —CH₃; j is 1; p is 1; s is 0; v is 0; x is 0; y is 2; z is 6; and, [symbol] corresponds to a double bond. In certain embodiments of Formula (P-II) and (II), L₁, L₂ are —(CH₂)₃—; M is —(CH₂)₃—; R^{d} is —H; R^{e} is —CH₃; j is 1; p is 1; s is 0; v is 0; x is 0; y is 2; z is 6; and, [symbol] corresponds to a double bond. In certain embodiments of Formula (P-II) and (II), L₁, L₂ are —(CH₂)₃—; M is —(CH₂)₄—; R^{d} is —H; R^{e} is —CH₃; j is 1; p is 1; s is 0; v is 0; x is 0; y is 2; z is 2; and, [symbol] corresponds to a double bond. In certain embodiments of Formula (P-II) and (II), L₁, L₂ are —(CH₂)₃—; M is —(CH₂)₅—; R^{d} is —H; R^{e} is —CH₃; j is 1; p is 1; s is 0; v is 0; x is 0; y is 2; z is 6, and, [symbol] corresponds to a double bond. In certain embodiments of Formula (P-II) and (II), L₁, L₂ are —(CH₂)₃—; M is —(CH₂)₆—; R^{d} is —H; R^{e} is —CH₃; j is 1; p is 1; s is 0; v is 0; x is 0; y is 2; z is 6; and, [symbol] corresponds to a double bond.

For example, in certain embodiments of Formula (P-II) and (II), L₁, L₂, K, and M are —CH₂—; R^{d} is —H; R^{e} is —H; j is 1; p is 1; s is 0; v is 0; x is 0; y is 2; z is 2; and, [symbol] corresponds to a double bond. In certain embodiments of Formula (P-II) and (II), L₁, L₂ are —(CH₂)₃—; K is —CH₂—; M is —CH₂—; R^{d} is —H; R^{e} is —CH₃; j is 1; p is 1; s is 0; v is 0; x is 0; y is 2; z is 2; and, [symbol] corresponds to a double bond. In certain embodiments of Formula (P-II) and (II), L₁, L₂ are —(CH₂)₃—; K is —CH₂—; M is —CH₂)₂—; R^{d} is —H; R^{e} is —CH₃; j is 1; p is 1; s is 0; v is 0; x is 0; y is 2; z is 2; and, [symbol] corresponds to a double bond. In certain embodiments of Formula (P-II) and (II), L₁, L₂ are —(CH₂)₃—; K is —CH₂—; M is —(CH₂)₃—; R^{d} is —H; R^{e} is —CH₃; j is 1; p is 1; s is 0; v is 0; x is 0; y is 2; z is 2; and, [symbol] corresponds to a double bond. In certain embodiments of Formula (P-II) and (II), L₁, L₂ are —(CH₂)₃—; K is —CH₂—; M is —(CH₂)₄—; R^{d} is —H; RV is —CH₃; j is 1; p is 1; s is 0; v is 0; x is 0; y is 2; z is 2; and, [symbol] corresponds to a double bond. In certain embodiments of Formula (P-II) and (II), L₁, L₂ are —(CH₂)₃—; K is —CH₂—; M is —(CH₂)₅—; R^{d} is —H; R^{e} is —CH₃; j is 1; p is 1; s is 0; v is 0; x is 0; y is 2; z is 2; and, [symbol] corresponds to a double bond. In certain embodiments of Formula (P-II) and (II), L₁, L₂ are —(CH₂)₃—; K is —CH₂—; M is —(CH₂)₆—; R^{d} is —H; R^{e} is —CH₃; j is 1; p is 1; s is 0; v is 0; x is 0; y is 2; z is 2; and, [symbol] corresponds to a double bond.

For example, in certain embodiments of Formula (P-II) and (II), L₁, L₂, K. and M are —CH₂—; R^{d} is —H; R^{e} is —H; j is 1; p is 1; s is 0; v is 0; x is 0; y is 2; z is 3; and, [symbol] corresponds to a double bond. In certain embodiments of Formula (P-II) and (II), L₁, L₂ are —(CH₂)₃—; K is —CH₂—; M is —CH₂—; R^{d} is —H; R^{e} is —CH₃; j is 1; p is 1; s is 0; v is 0; x is 0; y is 2; z is 3; and, [symbol] corresponds to a double bond. In certain embodiments of Formula (P-II) and (II), L₁, L₂ are —(CH₂)₃—; K is —CH₂—; M is —(CH₂)₂—; R^{d} is —H; R^{e} is —CH₃; j is 1; p is 1; s is 0; v is 0; x is 0; y is 2; z is 3; and, [symbol] corresponds to a double bond. In certain embodiments of Formula (P-II) and (II), L₁, L₂ are —(CH₂)₃—; K is —CH₂—; M is —(CH₂)₃—; R^{d} is —H; R^{e} is —CH₃; j is 1; p is 1; s is 0; v is 0; x is 0; y is 2; z is 3; and, [symbol] corresponds to a double bond. In certain embodiments of Formula (P-II) and (II), L₁, L₂ are —(CH₂)₃—; K is —CH₂—; M is —(CH₂)₄—; R^{d} is —H; R^{e} is —CH₃; j is 1; p is 1; s is 0; v is 0; x is 0; y is 2; z is 3; and, [symbol] corresponds to a double bond. In certain embodiments of Formula (P-II) and (II), L₁, L₂ are —(CH₂)₃—; K is —CH₂—; M is —(CH₂)₅—; R^{d} is —H; R^{e} is —CH₃; j is 1; p is 1; s is 0; v is 0; x is 0; y is 2; z is 3; and, [symbol] corresponds to a double bond. In certain embodiments of Formula (P-II) and (II), L₁, L₂ are —(CH₂)₃—; K is —CH₂—; M is —(CH₂)₆—; R^{d} is —H; R^{e} is —CH₃; j is 1; p is 1; s is 0; v is 0; x is 0; y is 2; z is 3; and, [symbol] corresponds to a double bond.

For example, in certain embodiments of Formula (P-II) and (II), L₁, L₂, K. and M are —CH₂—; R^{d} is —H; R^{e} is —H; j is 1; p is 1; s is 0; v is 0; x is 0; y is 2; z is 6; and, [symbol] corresponds to a double bond. In certain embodiments of Formula (P-II) and (II), L₁, L₂ are —(CH₂)₃—; K is —CH₂—; M is —CH₂—; R^{d} is —H; R^{e} is —CH₃; j is 1; p is 1; s is 0; v is 0; x is 0; y is 2; z is 6; and, [symbol] corresponds to a double bond. In certain embodiments of Formula (P-II) and (II), L₁, L₂ are —(CH₂)₃—; K is —CH₂—; M is —(CH₂)₂—; R^{d} is —H; R^{e} is —CH₃; j is 1; p is 1; s is 0; v is 0; x is 0; y is 2; z is 6; and, [symbol] corresponds to a double bond. In certain embodiments of Formula (P-II) and (II), L₁, L₂ are —(CH₂)₃—; K is —CH₂—; M is —(CH₂)₃—; R^{d} is —H; R^{e} is —CH₃; j is 1; p is 1; s is 0; v is 0; x is 0; y is 2; z is 6; and, [symbol] corresponds to a double bond. In certain embodiments of Formula (P-II) and (II), L₁, L₂ are —(CH₂)₃—; K is —CH₂—; M is —(CH₂)₄—; R^{d} is —H; R^{e} is —CH₃; j is 1; p is 1; s is 0; v is 0; x is 0; y is 2; z is 2; and, [symbol] corresponds to a double bond. In certain embodiments of Formula (P-II) and (II), L₁, L₂ are —(CH₂)₃—; K is —CH₂—; M is —(CH₂)₅—; R^{d} is —H; R^{e} is —CH₃; j is 1; p is 1; s is 0; v is 0; x is 0; y is 2; z is 6; and, [symbol] corresponds to a double bond. In certain embodiments of Formula (P-II) and (II), L₁, L₂ are —(CH₂)₃—; K is —CH₂—; M is —(CH₂)₆—; R^{d} is —H; R^{e} is —CH₃; j is 1; p is 1; s is 0; v is 0; x is 0; y is 2; z is 6; and, [symbol] corresponds to a double bond.

For example, in certain embodiments of Formula (P-II) and (II), L₁, L₂ and M are —CH₂CH₂—; R^{d} is —H; R^{e} is —H; j is 1; p is 1; s is 0; v is 0; x is 0; y is 2; z is 2; and, [symbol] corresponds to a double bond.

For example, in certain embodiments of Formula (P-II) and (II), L₁, L₂ and M are —CH₂CH₂CH₂—; R^{d} is —H; R^{e} is —H; j is 1; p is 1; s is 0; v is 0; x is 0; y is 2; z is 2; and, [symbol] corresponds to a double bond.

For example, in certain embodiments of Formula (P-II) and (II), L₁, L₂ and M are —CH₂—; R^{d} is —H; R^{e} is —CH₃; j is 1; p is 1; s is 0; v is 0; x is 0; y is 2; z is 2; and, [symbol] corresponds to a double bond.

For example, in certain embodiments of Formula (P-II) and (II), L₁, L₂ and M are —CH₂—; R^{d} is —H; R^{e} is —CH₂CH₃; j is 1; p is 1; s is 0; v is 0; x is 0; y is 2; z is 2; and, [symbol] corresponds to a double bond.

For example, in certain embodiments of Formula (P-II) and (II), L₁, L₂ and M are —CH₂—; R^{d} is —H; R^{e} is —H; j is 1; p is 2; s is 0; v is 0; x is 0; y is 2; z is 2; and, [symbol] corresponds to a double bond.

For example, in certain embodiments of Formula (P-II) and (II), L₁, L₂ and M are —CH₂—; R^{d} is —H; R^{e} is —H; j is 1; p is 1; s is 1; v is 0; x is 0; y is 2; z is 2; and, [symbol] corresponds to a double bond.

For example, in certain embodiments of Formula (P-II) and (II), L₁, L₂ and M are —CH₂—; R^{d} is —H; R^{e} is —H; j is 1; p is 1; s is 0; v is 0; x is 2; y is 2; z is 2; and, [symbol] corresponds to a double bond.

For example, in certain embodiments of Formula (P-II) and (II), L₁, L₂ and M are —CH₂—; R^{d} is —H; R^{e} is —H; j is 1; p is 1; s is 0; v is 0; x is 0; y is 2; z is 3; and, [symbol] corresponds to a double bond.

For example, in certain embodiments of Formula (P-II) and (II), L₁, L₂ and M are —CH₂—; R^{d} is —H; R^{e} is —H; j is 1; p is 1; s is 0; v is 0; x is 0; y is 2; z is 4; and, [symbol] corresponds to a double bond.

For example, in certain embodiments of Formula (P-II) and (II), L₁, L₂ and M are —CH₂—; R^{d} is —H; R^{e} is —H; j is 1; p is 1; s is 0; v is 0; x is 0; y is 2; z is 5; and, [symbol] corresponds to a double bond.

For example, in certain embodiments of Formula (P-II) and (II), L₁, L₂ and M are —CH₂—; R^{d} is —H; R^{e} is —H; j is 1; p is 1; s is 0; v is 1; x is 0; y is 2; z is 2; [G] is serine; and, [symbol] corresponds to a double bond.

For example, in certain embodiments of Formula (P-II) and (II), L₁, L₂ and M are —CH₂—; R^{d} is —H; R^{e} is —H; j is 1; p is 1; s is 0; v is 1; x is 0; y is 2; z is 2; [G] is threonine; and, [symbol] corresponds to a double bond.

For example, in certain embodiments of Formula (P-II) and (II), L₁, L₂ and M are —CH₂—; R^{d} is —H; R^{e} is —H; j is 1; p is 1; s is 0; v is 1; x is 0; y is 2; z is 2; [G] is aspartic acid; and, [symbol] corresponds to a double bond.

For example, in certain embodiments of Formula (P-II) and (II), L₁, L₂ and M are —CH₂—; R^{d} is —H; R^{e} is —H; j is 1; p is 1; s is 0; v is 1; x is 0; y is 2; z is 2; [G] is glutamic acid; and, [symbol] corresponds to a double bond.

For example, in certain embodiments of Formula (P-II) and (II), L₁, L₂ and M are —CH₂—; R^{d} is —H; R^{e} is —H; j is 1; p is 1; s is 0; v is 1; x is 0; y is 2; z is 2; [G] is asparagine; and, [symbol] corresponds to a double bond.

For example, in certain embodiments of Formula (P-II) and (II), L₁, L₂ and M are —CH₂—; R^{d} is —H; R^{e} is —H; j is 1; p is 1; s is 0; v is 1; x is 0; y is 2; z is 2; [G] is

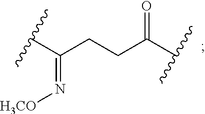 [SMILES: *C(=O)CC/C(*)=N/OC]

and, [symbol] corresponds to a double bond.

For example, in certain embodiments of Formula (P-II) and (II), L₁, L₂ and M are —CH₂—; R^{d} is —H; R^{e} is —H; j is 1p is 1; q is 0; s is 0; v is 0; y is 2; z is 2; and [symbol] corresponds to a double bond.

For example, in certain embodiments of Formula (P-II) and (II), L₁, L₂ and M are —CH₂—; R^{d} is —H; R^{e} is —H; j is 1; p is 1; q is 0; s is 0; y is 1; y is 2; z is 2; [G] is

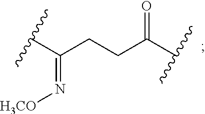 [SMILES: *C(=O)CC/C(*)=N/OC]

and, [symbol] corresponds to a double bond.

In any of the above embodiments, wherein [G] is a group of formula:

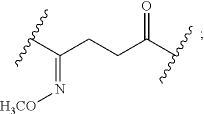 [SMILES: *C(=O)CC/C(*)=N/OC]

in certain embodiments, w is 0, and R^{a} is —C(CH₃)₃.

In any of the above embodiments, wherein [G] is a group of any one of the following formulae:

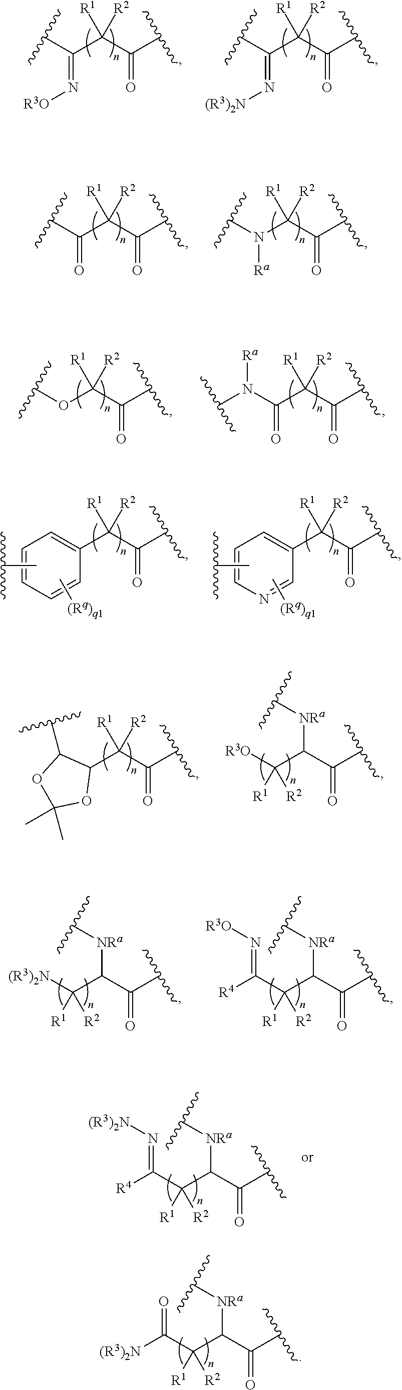

In any of the above embodiments, wherein

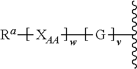

is of any one of the following formulae:

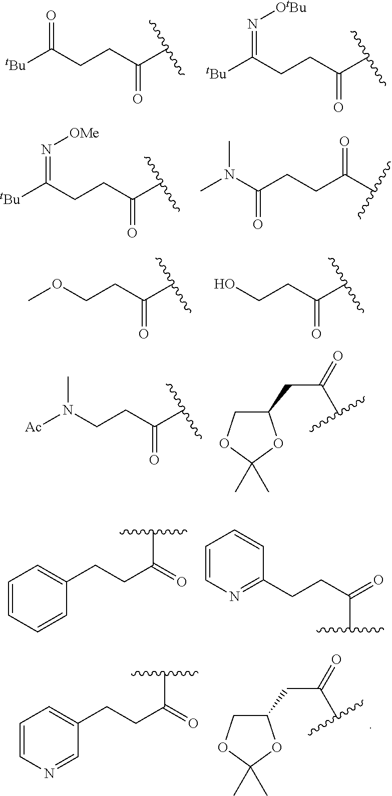

### Methods of Use

The present disclosure provides methods of treating a disease, disorder, or condition comprising administering to a subject diagnosed with or having susceptibility to the disease, disorder, or condition, a therapeutically effective amount of a stitched or stapled polypeptide as described herein, or pharmaceutically acceptable salt or stereoisomer thereof. Exemplary diseases, disorders, or conditions which may be treated by administration of a stitched or stapled polypeptide as described herein comprise proliferative, neurological, immunological, endocrinologic, cardiovascular, hematologic, and inflammatory diseases, disorders, or conditions, and conditions characterized by premature or unwanted cell death.

As used herein a proliferative disease, condition, or disorder includes, but is not limited to, cancer, hematopoietic neoplastic disorders, proliferative breast disease, proliferative disorders of the lung, proliferative disorders of the colon, proliferative disorders of the liver, and proliferative disorders of the ovary.

Examples of cancers treatable by the methods disclosed herein include carcinoma, sarcoma, or metastatic disorders, breast cancer, ovarian cancer, colon cancer, lung cancer, fibrosarcoma, myosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, gastric cancer, esophageal cancer, rectal cancer, pancreatic cancer, ovarian cancer, prostate cancer, uterine cancer, cancer of the head and neck, skin cancer, brain cancer, squamous cell carcinoma, sebaceous gland carcinoma, papillary carcinoma, papillary adenocarcinoma, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular cancer, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemia, lymphoma, or Kaposi sarcoma,

Examples of hematopoietic neoplastic disorders treatable by the above method includes diseases involving hyperplastic/neoplastic cells of hematopoietic origin, e.g., arising from myeloid, lymphoid or erythroid lineages, or precursor cells thereof. In certain embodiments, the diseases arise from poorly differentiated acute leukemias, e.g., erythroblastic leukemia and acute megakaryoblastic leukemia. Additional exemplary myeloid disorders include, but are not limited to, acute promyeloid leukemia (APML), acute myelogenous leukemia (AML) and chronic myelogenous leukemia (CML) (reviewed in Vaickus, L. (1991) Crit Rev in Oncol./Hemotol. 11:267-97); lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Stemberg disease.

Examples of proliferative breast disease treatable by the methods disclosed herein include epithelial hyperplasia, sclerosing adenosis, and small duct papillomas; tumors, e.g., stromal tumors such as fibroadenoma, phyllodes tumor, and sarcomas, and epithelial tumors such as large duct papilloma; carcinoma of the breast including in situ (noninvasive) carcinoma that includes ductal carcinoma in situ (including Paget's disease) and lobular carcinoma in situ, and invasive (infiltrating) carcinoma including, but not limited to, invasive ductal carcinoma, invasive lobular carcinoma, medullary carcinoma, colloid (mucinous) carcinoma, tubular carcinoma, and invasive papillary carcinoma, and miscellaneous malignant neoplasms. Disorders in the male breast include, but are not limited to, gynecomastia and carcinoma.

Examples of proliferative disorders of the lung treatable by the methods disclosed herein include, but are not limited to, bronchogenic carcinoma, including paraneoplastic syndromes, bronchioloalveolar carcinoma, neuroendocrine tumors, such as bronchial carcinoid, miscellaneous tumors, and metastatic tumors; pathologies of the pleura, including inflammatory pleural effusions, noninflammatory pleural effusions, pneumothorax, and pleural tumors, including solitary fibrous tumors (pleural fibroma) and malignant mesothelioma.

Examples of proliferative disorders of the colon treatable by the methods disclosed herein include, but are not limited to, non-neoplastic polyps, adenomas, familial syndromes, colorectal carcinogenesis, colorectal carcinoma, and carcinoid tumors.

Examples of proliferative disorders of the liver treatable by the methods disclosed herein include, but are not limited to, nodular hyperplasias, adenomas, and malignant tumors, including primary carcinoma of the liver and metastatic tumors.

Examples of proliferative disorders of the ovary treatable by the methods disclosed herein include, but are not limited to, ovarian tumors such as, tumors of coelomic epithelium, serous tumors, mucinous tumors, endometeriod tumors, clear cell adenocarcinoma, cystadenofibroma, brenner tumor, surface epithelial tumors; germ cell tumors such as mature (benign) teratomas, monodermal teratomas, immature malignant teratomas, dysgerminoma, endodermal sinus tumor, choriocarcinoma; sex cord-stomal tumors such as, granulosa-theca cell tumors, thecomafibromas, androblastomas, hill cell tumors, and gonadoblastoma; and metastatic tumors such as Krukenberg tumors.

The polypeptides described herein can also be used to treat, prevent or diagnose conditions charaterised by overactive cell death or cellular death due to physiologic insult etc. Some examples of conditions characterized by premature or unwanted cell death are or alternatively unwanted or excessive cellular proliferation include, but are not limited to hypocellular/hypoplastic, acellular/aplastic, or hypercellular/hyperplastic conditions. Some examples include hematologic disorders including but not limited to fanconi anemia, aplastic anemia, thalaessemia, congenital neutropenia, myelodysplasia. The polypeptides disclosed herein can be used to decrease apoptosis and can be used to treat disorders associated with an undesirable level of cell death. Thus, the anti-apoptotic of the peptides disclosed herein can be used to treat disorders such as those that lead to cell death associated with viral infection, e.g., associated with infection with human immunodeficiency virus (HIV).

The peptides disclosed herein can be used to treat disorders associated with undesirable cell death. A wide variety of neurological diseases are characterized by the gradual loss of specific sets of neurons, and the anti-apoptotic peptides can be used in the treatment of these disorders. Such disorders include Alzheimer's disease, Parkinson's disease, amyotrophic lateral sclerosis (ALS) retinitis pigmentosa, spinal muscular atrophy, and various forms of cerebellar degeneration. The cell loss in these diseases does not induce an inflammatory response, and apoptosis appears to be the mechanism of cell death. In addition, a number of hematologic diseases are associated with a decreased production of blood cells. These disorders include anemia associated with chronic disease, aplastic anemia, chronic neutropenia, and the myelodysplastic syndromes. Disorders of blood cell production, such as myelodysplastic syndrome and some forms of aplastic anemia, are associated with increased apoptotic cell death within the bone marrow. These disorders could result from the activation of genes that promote apoptosis, acquired deficiencies in stromal cells or hematopoietic survival factors, or the direct effects of toxins and mediators of immune responses. Two common disorders associated with cell death are myocardial infarctions and stroke. In both disorders, cells within the central area of ischemia, which is produced in the event of acute loss of blood flow, appear to die rapidly as a result of necrosis. However, outside the central ischemic zone, cells die over a more protracted time period and morphologically appear to die by apoptosis.

Some examples of neurologic disorders that can be treated with the polypeptides described herein include but are not limited to Alzheimer's Disease, Down's Syndrome, Dutch Type Hereditary Cerebral Hemorrhage Amyloidosis, Reactive Amyloidosis, Familial Amyloid Nephropathy with Urticaria and Deafness, Muckle-Wells Syndrome, Idiopathic Myeloma; Macroglobulinemia-Associated Myeloma. Familial Amyloid Polyneuropathy, Familial Amyloid Cardiomyopathy, Isolated Cardiac Amyloid, Systemic Senile Amyloidosis, Adult Onset Diabetes, Insulinoma, Isolated Atrial Amyloid, Medullary Carcinoma of the Thyroid, Familial Amyloidosis, Hereditary Cerebral Hemorrhage with Amyloidosis, Familial Amyloidotic Polyneuropathy, Scrapie, Creutzfeldt-Jacob Disease, Gerstmann Straussler-Scheinker Syndrome, Bovine Spongiform Encephalitis, a Prion-mediated disease, Huntington's Disease, Pick's Disease, Amyotrophic Lateral Sclerosis (ALS), Parkinson's Disease, and Lewy Body Disease.

Some examples of endocrinologic disorders that can be treated with the polypeptides described herein include, but are not limited to, diabetes, hypothyroidism, hypopituitarism, hypoparathyroidism, hypogonadism, fertility disorders, etc.

Some examples of immunologic disorders that can be treated with the polypeptides described herein include, but are not limited to, organ transplant rejection, arthritis, lupus, IBD, Crohn's disease, asthma, multiple sclerosis, diabetes, Graft versus host diseases, autoimmune diseases, psoriasis, rheumatoid arthritis, etc.

Examples of cardiovascular disorders that can be treated or prevented with the polypeptides described herein include, but are not limited to, atherosclerosis, myocardial infarction, stroke, thrombosis, aneurism, heart failure, ischemic heart disease, angina pectoris, sudden cardiac death, hypertensive heart disease; non-coronary vessel disease, such as arteriolosclerosis, small vessel disease, nephropathy, hypertriglyceridemia, hypercholesterolemia, hyperlipidemia, xanthomatosis, asthma, hypertension, emphysema and chronic pulmonary disease; or a cardiovascular condition associated with interventional procedures (“procedural vascular trauma”), such as restenosis following angioplasty, placement of a shunt, stent, synthetic or natural excision grafts, indwelling catheter, valve or other implantable devices.

The stapled and stitched polypeptides provides herein can treat the above-described diseases, disorders, or conditions, for instance, by disrupting native protein-protein, protein-ligand, and/or protein-receptor interactions. For example, many biologically important protein/protein interactions, such as p53/MDM2 and Bcl-X1/Bak, are mediated by one protein donating a helix into a cleft of its helix-accepting partner. The interaction of p53 and MDM2 and mutations in the p53 gene have been identified in virtually half of all reported cancer cases (see, Shair Chem. & Biol. 1997, 4, 791, the entire contents of which are incorporated herein by reference). As stresses are imposed on a cell, p53 is believed to orchestrate a response that leads to either cell-cycle arrest and DNA repair, or programmed cell death. As well as mutations in the p53 gene that alter the function of the p53 protein directly, p53 can be altered by changes in MDM2. The MDM2 protein has been shown to bind to p53 and disrupt transcriptional activation by associating with the transactivation domain of p53. For example, an 11 amino-acid peptide derived from the transactivation domain of p53 forms an amphipathic alpha-helix of 2.5 turns that inserts into the MDM2 crevice.

Thus, in certain embodiments, a stitched or stapled polypeptide as described herein is an alpha helical polypeptide that is capable of binding tightly to a helix acceptor and disrupting native protein/protein interactions. These structures may then be screened using high throughput techniques to identify optimal small molecule peptides. In certain embodiments, a stitched or stapled polypeptide as described herein is an alpha helical p53 polypeptide capable of binding to the Xenopus MDM2 protein. The novel structures that disrupt the MDM2 interaction might be useful for many applications, including, but not limited to, control of soft tissue sarcomas (which overexpresses MDM2 in the presence of wild type p53). These cancers may be held in check with small molecules that could intercept MDM2, thereby preventing suppression of p53. Additionally, small molecules disrupters of MDM2-p53 interactions could be used as adjuvant therapy to help control and modulate the extent of the p53 dependent apoptosis response in conventional chemotherapy.

In certain embodiments, polypeptides disclosed herein are homologous to a known alpha helical peptide. In certain embodiments, the inventive polypeptide is at least 80%, 85%, 90%, or 95% homologous to a known alpha helical peptide.

In addition, polypeptides disclosed herein may be useful in the area of materials science. For example, molecules such as lipids and other polymeric molecules may be attached to the terminal peptide moieties and thus generate potentially important biomaterials.

In addition to the above-mentioned uses, polypeptides disclosed herein may be used for studies in bioinorganic chemistry or in catalysis, either as a ligand for a transition metal capable of mimicking an important biological environment, or by acting in concert with a particular transition metal catalyst to effect a desired chemical reaction.

### Pharmaceutical Compositions

The present disclosure provides pharmaceutical compositions comprising a stitched or stapled polypeptide as described herein and, optionally, a pharmaceutically acceptable excipient. Such pharmaceutical compositions may optionally comprise one or more additional biologically-active substances. In accordance with some embodiments, a method of administering a pharmaceutical composition to a subject in need thereof is provided. In some embodiments, pharmaceutical compositions are administered to humans. For the purposes of the present disclosure, the phrase “active ingredient” generally refers to a stitched or stapled polypeptide as described herein.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions as described herein is contemplated include, but are not limited to, humans and/or other primates; mammals, including commercially relevant mammals such as cattle, pigs, horses, sheep, cats, and/or dogs; and/or birds, including commercially relevant birds such as chickens, ducks, geese, and/or turkeys.

The formulations of the pharmaceutical compositions described herein may be prepared by any method known or hereafter developed in the art of pharmacology. In general, such preparatory methods include the step of bringing the active ingredient into association with a carrier and/or one or more other accessory ingredients, and then, if necessary and/or desirable, shaping and/or packaging the product into a desired single- or multi-dose unit.

A pharmaceutical composition may be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

The relative amounts of the active ingredient, the pharmaceutically acceptable carrier, and/or any additional ingredients in a pharmaceutical composition of the disclosure will vary, depending upon the identity, size, and/or condition of the subject treated and further depending upon the route by which the composition is to be administered. By way of example, the composition may comprise between 0.1% and 100% (w/w) active ingredient.

Pharmaceutical formulations may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21^{st} Edition. A. R. Gennaro, (Lippincott, Williams & Wilkins, Baltimore, MD, 2006) discloses various carriers used in formulating pharmaceutical compositions and known techniques for the preparation thereof. Except insofar as any conventional carrier medium is incompatible with a substance or its derivatives, such as by producing any undesirable biological effect or otherwise interacting in a deleterious manner with any other component(s) of the pharmaceutical composition, its use is contemplated to be within the scope of this disclosure.

In some embodiments, the pharmaceutically acceptable excipient is at least 95%, 96%, 97%, 98%, 99%, or 100% pure. In some embodiments, the excipient is approved for use in humans and for veterinary use. In some embodiments, the excipient is approved by United States Food and Drug Administration. In some embodiments, the excipient is pharmaceutical grade. In some embodiments, the excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in the inventive formulations. Excipients such as cocoa butter and suppository waxes, coloring agents, coating agents, sweetening, flavoring, and perfuming agents can be present in the composition, according to the judgment of the formulator.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxy propyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof.

Exemplary binding agents include, but are not limited to, starch (e.g. cornstarch and starch paste); gelatin; sugars (e.g. sucrose, glucose, dextrose, dextrin, molasses, lactose, lactitol, mannitol,); natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan); alginates; polyethylene oxide; polyethylene glycol; inorganic calcium salts; silicic acid; polymethacrylates; waxes; water; alcohol; etc.; and combinations thereof.

Exemplary preservatives may include antioxidants, chelating agents, antimicrobial preservatives, antifungal preservatives, alcohol preservatives, acidic preservatives, and other preservatives. Exemplary antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and sorbic acid. Exemplary alcohol preservatives include, but are not limited to, ethanol, polyethylene glycol, phenol, phenolic compounds, bisphenol, chlorobutanol, hydroxybenzoate, and phenylethyl alcohol. Exemplary acidic preservatives include, but are not limited to, vitamin A, vitamin C, vitamin E, beta-carotene, citric acid, acetic acid, dehydroacetic acid, ascorbic acid, sorbic acid, and phytic acid. Other preservatives include, but are not limited to, tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben 11, Neolone, Kathon, and Euxyl. In certain embodiments, the preservative is an anti-oxidant. In other embodiments, the preservative is a chelating agent.

Exemplary buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

Exemplary lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Liquid dosage forms for oral and parenteral administration include, but are not limited to, pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the polypeptides of the disclosure are mixed with solubilizing agents such as Cremophor, alcohols, oils, modified oils, glycols, polysorbates, cyclodextrins, polymers, and combinations thereof.

Injectable preparations, for example, sterile injectable aqueous or oleaginous suspensions may be formulated according to the known art using suitable dispersing or wetting agents and suspending agents. The sterile injectable preparation may be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Compositions for rectal or vaginal administration are typically suppositories which can be prepared by mixing the polypeptides of the disclosure with suitable non-irritating excipients or carriers such as cocoa butter, polyethylene glycol or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may comprise buffering agents.

Solid compositions of a similar type may be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes. Solid compositions of a similar type may be employed as fillers in soft and hard-filled gelatin capsules using such excipients as lactose or milk sugar as well as high molecular weight polyethylene glycols and the like.

The active ingredients can be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active ingredient may be admixed with at least one inert diluent such as sucrose, lactose or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes.

Dosage forms for topical and/or transdermal administration of a polypeptide of the disclosure may include ointments, pastes, creams, lotions, gels, powders, solutions, sprays, inhalants and/or patches. Generally, the active component is admixed under sterile conditions with a pharmaceutically acceptable carrier and/or any needed preservatives and/or buffers as may be required. Additionally, the present disclosure contemplates the use of transdermal patches, which often have the added advantage of providing controlled delivery of an active ingredient to the body. Such dosage forms may be prepared, for example, by dissolving and/or dispensing the active ingredient in the proper medium. Alternatively or additionally, the rate may be controlled by either providing a rate controlling membrane and/or by dispersing the active ingredient in a polymer matrix and/or gel.

Suitable devices for use in delivering intradermal pharmaceutical compositions described herein include short needle devices such as those described in U.S. Pat. Nos. 4,886,499; 5,190,521; 5,328,483; 5,527,288; 4,270,537; 5,015,235; 5,141,496; and 5,417,662. Intradermal compositions may be administered by devices which limit the effective penetration length of a needle into the skin, such as those described in PCT publication WO 99/34850 and functional equivalents thereof. Jet injection devices which deliver liquid vaccines to the dermis via a liquid jet injector and/or via a needle which pierces the stratum corneum and produces a jet which reaches the dermis are suitable. Jet injection devices are described, for example, in U.S. Pat. Nos. 5,480,381; 5,599,302; 5,334,144; 5,993,412; 5,649,912; 5,569,189; 5,704,911; 5,383,851; 5,893,397; 5,466,220; 5,339,163; 5,312,335; 5,503,627; 5,064,413; 5,520,639; 4,596,556; 4,790,824; 4,941,880; 4,940,460; and PCT publications WO 97/37705 and WO 97/13537. Ballistic powder/particle delivery devices which use compressed gas to accelerate vaccine in powder form through the outer layers of the skin to the dermis are suitable. Alternatively or additionally, conventional syringes may be used in the classical mantoux method of intradermal administration.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions. Topically-administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

A pharmaceutical composition of the disclosure may be prepared, packaged, and/or sold in a formulation suitable for pulmonary administration via the buccal cavity. Such a formulation may comprise dry particles which comprise the active ingredient and which have a diameter in the range from about 0.5 to about 7 nanometers or from about 1 to about 6 nanometers. Such compositions are conveniently in the form of dry powders for administration using a device comprising a dry powder reservoir to which a stream of propellant may be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions as described herein formulated for pulmonary delivery may provide the active ingredient in the form of droplets of a solution and/or suspension. Such formulations may be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

The formulations described herein as being useful for pulmonary delivery are useful for intranasal delivery of a pharmaceutical composition of the disclosure. Another formulation suitable for intranasal administration is a coarse powder comprising the active ingredient and having an average particle from about 0.2 to 500 micrometers. Such a formulation is administered in the manner in which snuff is taken, i.e. by rapid inhalation through the nasal passage from a container of the powder held close to the nares.

Formulations suitable for nasal administration may, for example, comprise from about as little as 0.1% (w/w) and as much as 100% (w/w) of the active ingredient, and may comprise one or more of the additional ingredients described herein. A pharmaceutical composition of the disclosure may be prepared, packaged, and/or sold in a formulation suitable for buccal administration. Such formulations may, for example, be in the form of tablets and/or lozenges made using conventional methods, and may, for example, 0.1 to 20% (w/w) active ingredient, the balance comprising an orally dissolvable and/or degradable composition and, optionally, one or more of the additional ingredients described herein. Alternately, formulations suitable for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition may be prepared, packaged, and/or sold in a formulation suitable for ophthalmic administration. Such formulations may, for example, be in the form of eye drops including, for example, a 0.1/1.0% (w/w) solution and/or suspension of the active ingredient in an aqueous or oily liquid carrier. Such drops may further comprise buffering agents, salts, and/or one or more other of the additional ingredients described herein. Other ophthalmically-administrable formulations which are useful include those which comprise the active ingredient in microcrystalline form and/or in a liposomal preparation. Ear drops and/or eye drops are contemplated as being within the scope of this disclosure.

General considerations in the formulation and/or manufacture of pharmaceutical agents may be found, for example, in Remington: The Science and Practice of Pharmacy 21^{st} ed., Lippincott Williams & Wilkins, 2005.

### Administration

In some embodiments, a therapeutically effective amount of a pharmaceutical composition as described herein is delivered to a patient and/or organism prior to, simultaneously with, and/or after diagnosis with a disease, disorder, and/or condition. In some embodiments, a therapeutic amount of a pharmaceutical composition as described herein is delivered to a patient and/or organism prior to, simultaneously with, and/or after onset of symptoms of a disease, disorder, and/or condition. In some embodiments, the amount of the stitched or stapled polypeptide as described herein is sufficient to treat, alleviate, ameliorate, relieve, delay onset of, inhibit progression of, reduce severity of, and/or reduce incidence of one or more symptoms or features of the disease, disorder, and/or condition.

The compositions, as disclosed herein, may be administered using any amount and any route of administration effective for treatment. The exact amount required will vary from subject to subject, depending on the species, age, and general condition of the subject, the severity of the infection, the particular composition, its mode of administration, its mode of activity, and the like. The pharmaceutical compositions as described herein are typically formulated in dosage unit form for ease of administration and uniformity of dosage. It will be understood, however, that the total daily usage of the pharmaceutical compositions as described herein will be decided by the attending physician within the scope of sound medical judgment. The specific therapeutically effective dose level for any particular subject or organism will depend upon a variety of factors including the disorder being treated and the severity of the disorder; the activity of the specific active ingredient employed; the specific composition employed; the age, body weight, general health, sex and diet of the subject; the time of administration, route of administration, and rate of excretion of the specific active ingredient employed; the duration of the treatment; drugs used in combination or coincidental with the specific active ingredient employed; and like factors well known in the medical arts.

The pharmaceutical compositions as described herein may be administered by any route. In some embodiments, the pharmaceutical compositions as described herein are administered variety of routes, including oral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, enteral, sublingual; by intratracheal instillation, bronchial instillation, and/or inhalation; and/or as an oral spray, nasal spray, and/or aerosol. Specifically contemplated routes are systemic intravenous injection, regional administration via blood and/or lymph supply, and/or direct administration to an affected site. In general the most appropriate route of administration will depend upon a variety of factors including the nature of the agent (e.g., its stability in the environment of the gastrointestinal tract), the condition of the subject (e.g., whether the subject is able to tolerate oral administration), etc. At present the oral and/or nasal spray and/or aerosol route is most commonly used to deliver therapeutic agents directly to the lungs and/or respiratory system. However, the disclosure embraces the delivery of the pharmaceutical compositions as described herein by any appropriate route taking into consideration likely advances in the sciences of drug delivery.

In certain embodiments, pharmaceutical compositions comprising the peptides disclosed herein may be administered at dosage levels sufficient to deliver from about 0.001 mg/kg to about 100 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic effect. The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations).

In some embodiments, the disclosure encompasses “therapeutic cocktails” comprising the polypeptides disclosed herein. In some embodiments, the polypeptide comprises a single species which can bind to multiple targets. In some embodiments, the polypeptides disclosed herein comprise different targeting moiety species, and all of the different targeting moiety species can bind to the same target. In some embodiments, different polypeptides comprise different targeting moiety species, and all of the different targeting moiety species can bind to different targets. In some embodiments, such different targets may be associated with the same cell type. In some embodiments, such different targets may be associated with different cell types.

It will be appreciated that the polypeptides and pharmaceutical compositions as described herein can be employed in combination therapies. The particular combination of therapies (therapeutics or procedures) to employ in a combination regimen will take into account compatibility of the desired therapeutics and/or procedures and the desired therapeutic effect to be achieved. It will be appreciated that the therapies employed may achieve a desired effect for the same purpose (for example, stitched or stapled polypeptide as described herein may be useful for detecting tumors and may be administered concurrently with another agent useful for detecting tumors), or they may achieve different effects (e.g., control of any adverse effects).

Pharmaceutical compositions as described herein may be administered either alone or in combination with one or more other therapeutic agents. By “in combination with,” it is not intended to imply that the agents must be administered at the same time and/or formulated for delivery together, although these methods of delivery are within the scope of the disclosure. The compositions can be administered concurrently with, prior to, or subsequent to, one or more other desired therapeutics or medical procedures. In general, each agent will be administered at a dose and/or on a time schedule determined for that agent. Additionally, the disclosure encompasses the delivery of the a pharmaceutical composition as described herein in combination with agents that may improve their bioavailability, reduce and/or modify their metabolism, inhibit their excretion, and/or modify their distribution within the body.

The particular combination of therapies (therapeutics and/or procedures) to employ in a combination regimen will take into account compatibility of the desired therapeutics and/or procedures and/or the desired therapeutic effect to be achieved. It will be appreciated that the therapies employed may achieve a desired effect for the same disorder (for example, a stitched or stapled polypeptide as described herein may be administered concurrently with another biologically active agent used to treat the same disorder), and/or they may achieve different effects (e.g., control of any adverse effects). In some embodiments, polypeptides of the disclosure are administered with a second biologically active agent that is approved by the U.S. Food and Drug Administration.

In will further be appreciated that biologically active agents utilized in this combination may be administered together in a single composition or administered separately in different compositions.

In general, it is expected that biologically active agents utilized in combination be utilized at levels that do not exceed the levels at which they are utilized individually. In some embodiments, the levels utilized in combination will be lower than those utilized individually.

In some embodiments, a pharmaceutical composition as described herein may be administered in combination with any biologically active agent or therapeutic regimen that is useful to treat, alleviate, ameliorate, relieve, delay onset of, inhibit progression of, reduce severity of, and/or reduce incidence of one or more symptoms or features of cancer. For example, pharmaceutical compositions may be administered in combination with traditional cancer therapies including, but not limited to, surgery, chemotherapy, radiation therapy, hormonal therapy, immunotherapy, complementary or alternative therapy, and any combination of these therapies.

In some embodiments, pharmaceutical compositions are administered in combination with surgery to remove a tumor. Because complete removal of a tumor with minimal or no damage to the rest of a patient's body is typically the goal of cancer treatment, surgery is often performed to physically remove part or all of a tumor. If surgery is unable to completely remove a tumor, additional therapies (e.g. chemotherapy, radiation therapy, hormonal therapy, immunotherapy, complementary or alternative therapy) may be employed.

In some embodiments, pharmaceutical compositions are administered in combination with radiation therapy. Radiation therapy (also known as radiotherapy, X-ray therapy, or irradiation) is the use of ionizing radiation to kill cancer cells and shrink tumors. Radiation therapy may be used to treat almost any type of solid tumor, including cancers of the brain, breast, cervix, larynx, lung, pancreas, prostate, skin, stomach, uterus, or soft tissue sarcomas. Radiation can be used to treat leukemia and lymphoma. Radiation therapy can be administered externally via external beam radiotherapy (EBRT) or internally via brachytherapy. Typically, the effects of radiation therapy are localized and confined to the region being treated. Radiation therapy injures or destroys tumor cells in an area being treated (e.g. a target organ, tissue, and/or cell) by damaging their genetic material, preventing tumor cells from growing and dividing. In general, radiation therapy attempts to damage as many tumor cells as possible while limiting harm to nearby healthy tissue. Hence, it is often administered in multiple doses, allowing healthy tissue to recover between fractions.

In some embodiments, pharmaceutical compositions are administered in combination with immunotherapy. Immunotherapy is the use of immune mechanisms against tumors which can be used in various forms of cancer, such as breast cancer (e.g. trastuzumab/Herceptin®), leukemia (e.g. gemtuzumab ozogamicin/Mylotarg®), and non-Hodgkin's lymphoma (e.g. rituximab/Rituxan®). In some embodiments, immunotherapy agents are monoclonal antibodies directed against proteins that are characteristic to the cells of the cancer in question. In some embodiments, immunotherapy agents are cytokines that modulate the immune system's response. In some embodiments, immunotherapy agents may be vaccines.

In some embodiments, vaccines can be administered to prevent and/or delay the onset of cancer. In some embodiments, cancer vaccines prevent and/or delay the onset of cancer by preventing infection by oncogenic infectious agents. In some embodiments, cancer vaccines prevent and/or delay the onset of cancer by mounting an immune response against cancer-specific epitopes. To give but one example of a cancer vaccine, an experimental vaccine for HPV types 16 and 18 was shown to be 100% successful at preventing infection with these types of HPV and, thus, are able to prevent the majority of cervical cancer cases (Harper et al., 2004, Lancet, 364:1757).

In some embodiments, pharmaceutical compositions are administered in combination with complementary and alternative medicine treatments. Some exemplary complementary measures include, but are not limited to, botanical medicine (e.g. use of mistletoe extract combined with traditional chemotherapy for the treatment of solid tumors); acupuncture for managing chemotherapy-associated nausea and vomiting and in controlling pain associated with surgery; prayer; psychological approaches (e.g. “imaging” or meditation) to aid in pain relief or improve mood. Some exemplary alternative measures include, but are not limited to, diet and other lifestyle changes (e.g. plant-based diet, the grape diet, and the cabbage diet).

In some embodiments, pharmaceutical compositions are administered in combination with any of the traditional cancer treatments described herein, which are often associated with unpleasant, uncomfortable, and/or dangerous side effects. For example, chronic pain often results from continued tissue damage due to the cancer itself or due to the treatment (i.e., surgery, radiation, chemotherapy). Alternatively or additionally, such therapies are often associated with hair loss, nausea, vomiting, diarrhea, constipation, anemia, malnutrition, depression of immune system, infection, sepsis, hemorrhage, secondar neoplasms, cardiotoxicity, hepatotoxicity, nephrotoxicity, ototoxicity, etc. Thus, pharmaceutical compositions which are administered in combination with any of the traditional cancer treatments described herein may be also be administered in combination with any therapeutic agent or therapeutic regimen that is useful to treat, alleviate, ameliorate, relieve, delay onset of, inhibit progression of, reduce severity of, and/or reduce incidence of one or more side effects of cancer treatment. To give but a few examples, pain can be treated with opioids and/or analgesics (e.g. morphine, oxycodone, antiemetics, etc.); nausea and vomiting can be treated with 5-HT₃ inhibitors (e.g. dolasetron/Anzemet®, granisetron/Kytril®, ondansetron/Zofran®, palonsetron/Aloxi®) and/or substance P inhibitors (e.g. aprepitant/Emend®); immunosuppression can be treated with a blood transfusion; infection and/or sepsis can be treated with antibiotics (e.g. penicillins, tetracyclines, cephalosporins, sulfonamides, aminoglycosides, etc.); and so forth.

In some embodiments, pharmaceutical compositions may be administered and/or inventive diagnostic methods may be performed in combination with any therapeutic agent or therapeutic regimen that is useful to diagnose one or more symptoms or features of cancer (e.g. detect the presence of and/or locate a tumor). In some embodiments, the stitched or stapled polypeptide as described herein may be used in combination with one or more other diagnostic agents. To give but one example, polypeptides used to detect tumors may be administered in combination with other agents useful in the detection of tumors. For example, the stitched or stapled polypeptide as described herein may be administered in combination with traditional tissue biopsy followed by immunohistochemical staining and serological tests (e.g. prostate serum antigen test). Alternatively or additionally, the stitched or stapled polypeptide as described herein may be administered in combination with a contrasting agent for use in computed tomography (CT) scans and/or MRI.

### Kits

The disclosure provides a variety of kits comprising one or more of the polypeptides disclosed herein. For example, the disclosure provides a kit comprising a stitched or stapled polypeptide as described herein and instructions for use. A kit may comprise multiple different polypeptides. A kit may comprise any of a number of additional components or reagents in any combination. All of the various combinations are not set forth explicitly but each combination is included in the scope of the disclosure

According to certain embodiments of the disclosure, a kit may include, for example, (i) one or more polypeptides and one or more particular biologically active agents to be delivered; (ii) instructions for administering the polypeptide to a subject in need thereof.

Kits typically include instructions which may, for example, comprise protocols and/or describe conditions for production of the polypeptides, administration of the polypeptides to a subject in need thereof, design of the polypeptides, etc. Kits will generally include one or more vessels or containers so that some or all of the individual components and reagents may be separately housed. Kits may also include a means for enclosing individual containers in relatively close confinement for commercial sale, e.g., a plastic box, in which instructions, packaging materials such as styrofoam, etc., may be enclosed. An identifier, e.g., a bar code, radio frequency identification (ID) tag, etc., may be present in or on the kit or in or one or more of the vessels or containers included in the kit. An identifier can be used, e.g., to uniquely identify the kit for purposes of quality control, inventory control, tracking, movement between workstations, etc.

### EXAMPLES

These and other aspects of the present invention will be further appreciated upon consideration of the following Examples, which are intended to illustrate certain particular embodiments of the invention but are not intended to limit its scope, as defined by the claims.

### Example 1: Pro-Locked Stapled Peptides

### Materials and Methods

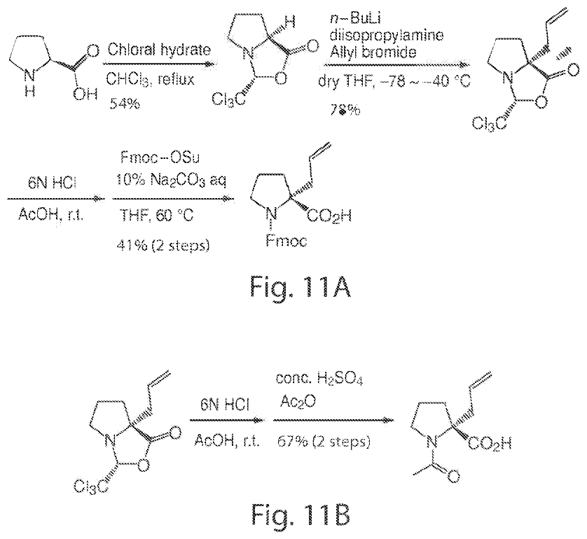

(R)—N-(Acetyl)-2-(2′-propenyl)proline (“P_{R3}”), a novel compound, was synthesized via modification of a reported synthetic route, followed by acetylation (Synlett, 1999, 1, 33-36; Tetrahedron, 2005, 61, 10018-10035). A scheme for the synthesis of P_{R3} is shown in FIG. 11. (R)—N-[(9H-Fluoren-9-ylmethoxy)carbonyl]-2-(2′-propenyl)proline could be used instead of (R)—N-(Acetyl)-2-(2′-propenyl)proline (“P_{R3}”).

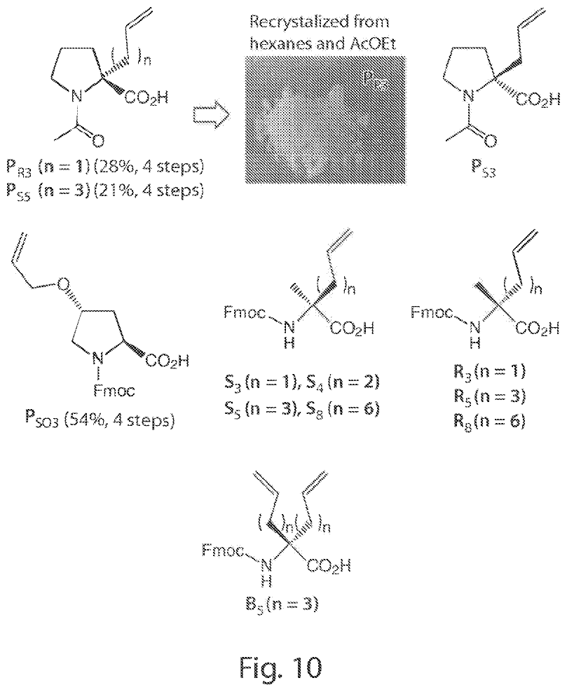

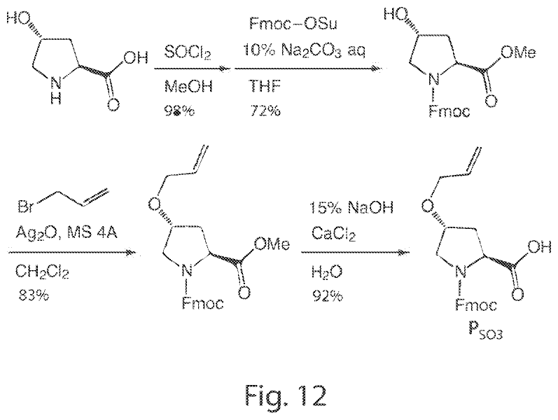

The compound P_{SO3} (See FIG. 10) allows for the synthesis of linkers originating from a position other than the alpha-carbon. A scheme for the synthesis of P_{SO3} is shown in FIG. 12. The scheme includes the steps of methyl-esterification, Fmoc protection, introduction of an allyl group and deprotection of a Fmoc group.

The compound P_{S3} was synthesized from D-proline following the synthetic scheme for preparation of the compound P_{SO3}.

The compound P_{S5} was synthesized following the synthetic scheme for preparation of the compound P_{R3} by replacing allyl bromide with 1-iodo-5-pentene.

(S)—N-[(9H-Fluoren-9-ylmethoxy)carbonyl]-2-(2′-propylenyl)alanine was purchased from Okeanos Tech Co.

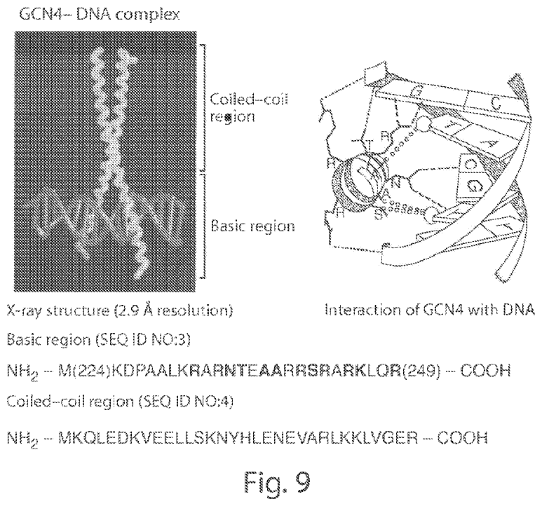

The GCN4 basic region was used as a test system to investigate the properties of the Pro-locked peptides, because the GCN4 basic region has a canonical nucleating (N-cap) sequence (N-DPAAL-C(SEQ ID NO: 39)) at the N-terminus of its DNA-recognition α-helix. (See FIG. 9)

### Generation of Pro-Locked Stapled Peptides

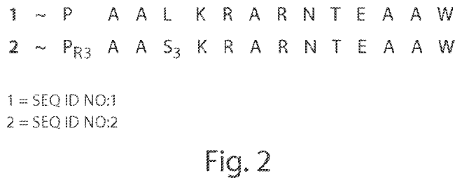

The peptides shown in FIG. 2 were synthesized manually, using solid phase conditions, rink amide MBHA resin (100~200 meshes) (Novabiochem), and Fmoc main-chain protecting group chemistry.

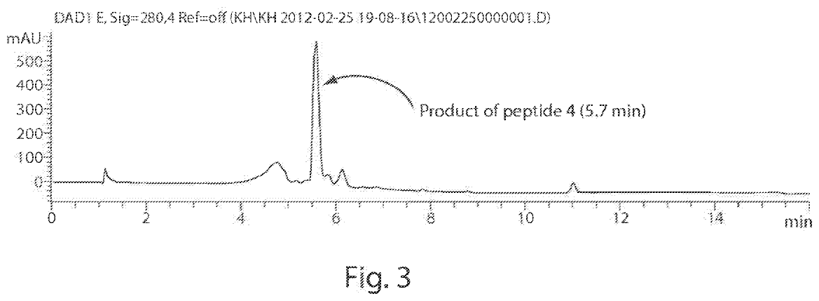

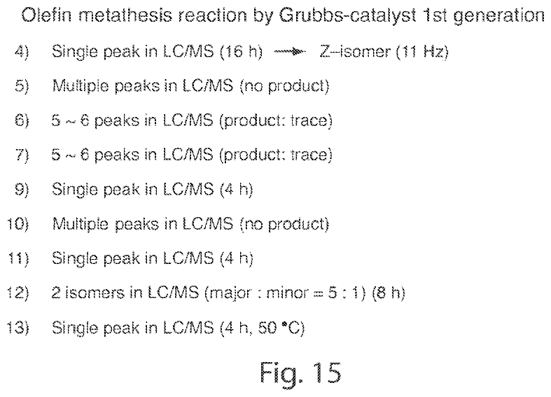

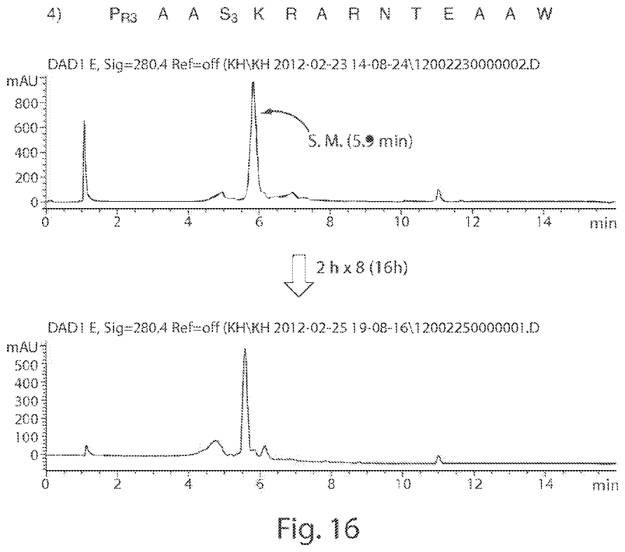

A crosslink was produced through an olefin-metathesis reaction between P_{R3} and S₃ (See FIG. 15). The olefin-metathesis reaction between P_{R3} and S₃ by Grubbs 1st generation catalyst proceeded completely after 16 h and a single product was observed in LC/MS (FIG. 3 and FIG. 16). The geometry of the generated olefin group in peptide 2 was determined to be Z-isomer by NMR measurement (the coupling constant between two olefinic protons is 11.0 Hz).

FIG. 10 provides additional amino acids and amino acid derivatives that were used in the generation of the Pro-locked stapled peptides described herein.

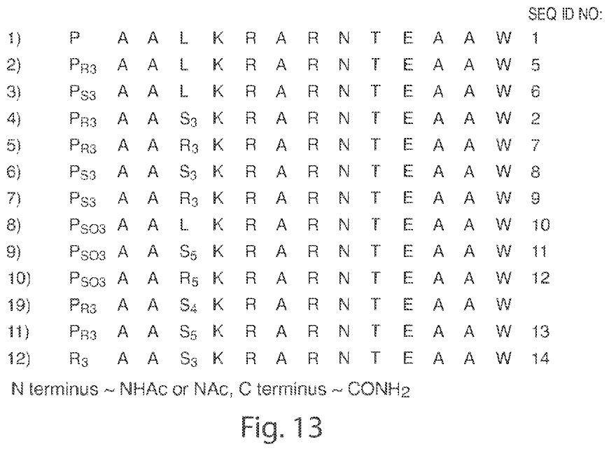

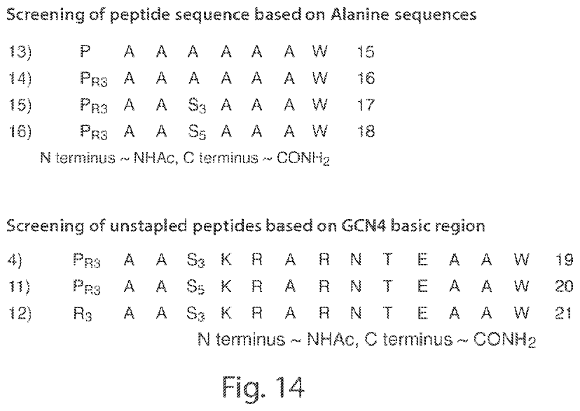

FIGS. 13 and 14 provide additional Pro-locked stapled peptides generated according to the methods provided herein. Non-cross-linked control peptides are provided in FIG. 14.

### Helicity and Stability of Pro-Locked Peptides

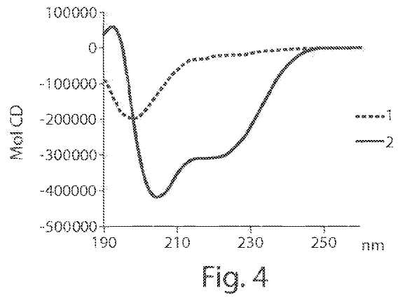

The conformation of peptide 1 and 2 was investigated by CD measurements (FIG. 4). The data show that peptide 1 adopts a random-coil and peptide 2 adopts an α-helix conformation at 20° C. The % helicity of peptide 2 is 67% at 20° C. The peptide having S₅ rather than S₃ still adopts an α-helix conformation at 20° C., but the % helicity is reduced to 44%.

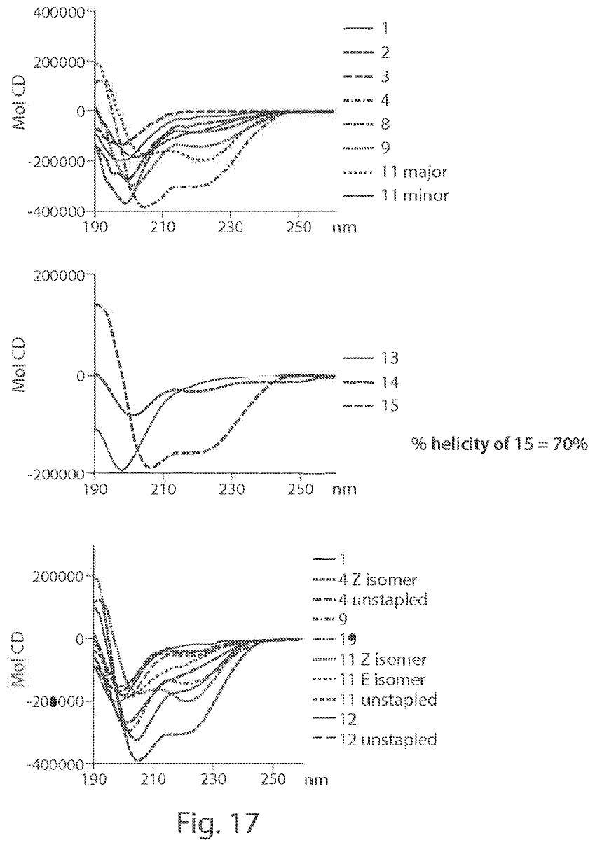

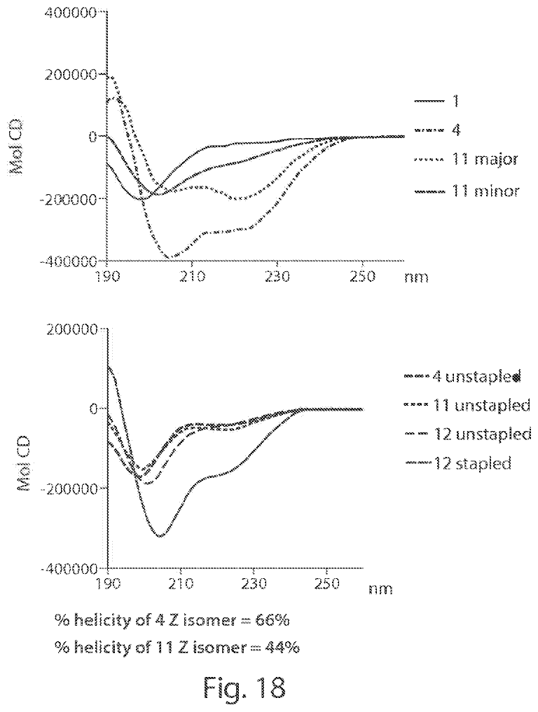

The conformation of additional pro-locked peptides as determined by CD measurements is provided in FIG. 17 and FIG. 18. (Peptide 1 of FIG. 2 corresponds to Peptide “1)” of FIG. 17, while Peptide 2 of FIG. 2 corresponds to Peptide “4)” of FIG. 17).

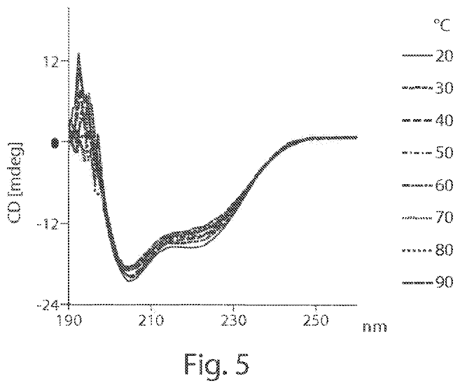

The stability of the Pro-locked peptide 2 was investigated by variable temperature CD measurement, and it was found that the α-helix conformation was completely maintained in the range from 20° C. to 90° C. in 50 mM sodium phosphate buffer (pH 8.0), alone (FIG. 5) and with 100 mM NaCl, 1 M NaCl, and 10% TFE. These observations indicate that the proline stapled peptides adopt an α-helix conformation with extraordinary stability.

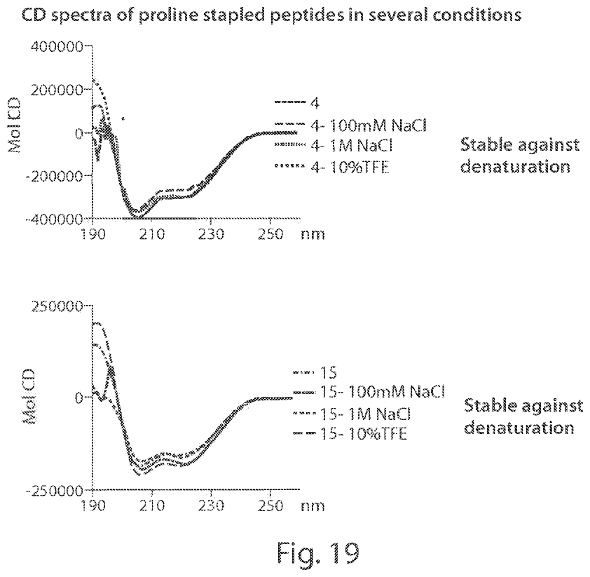

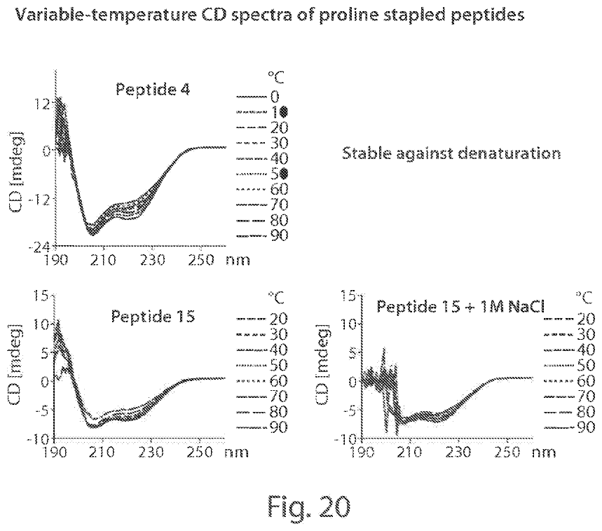

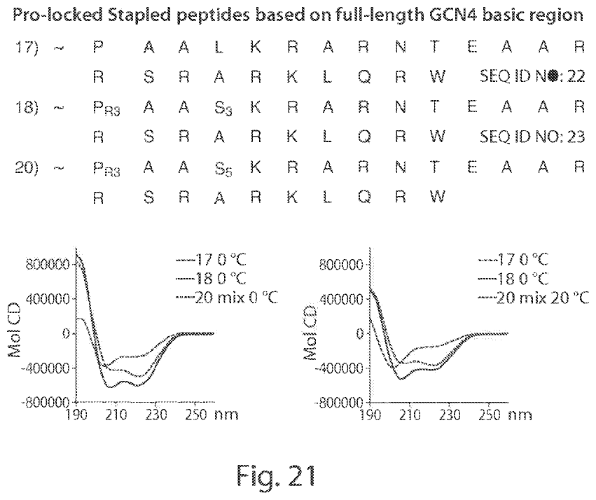

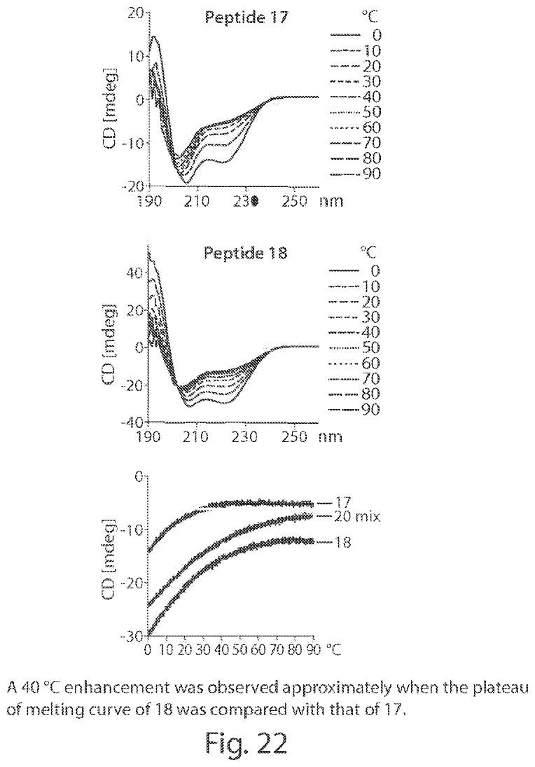

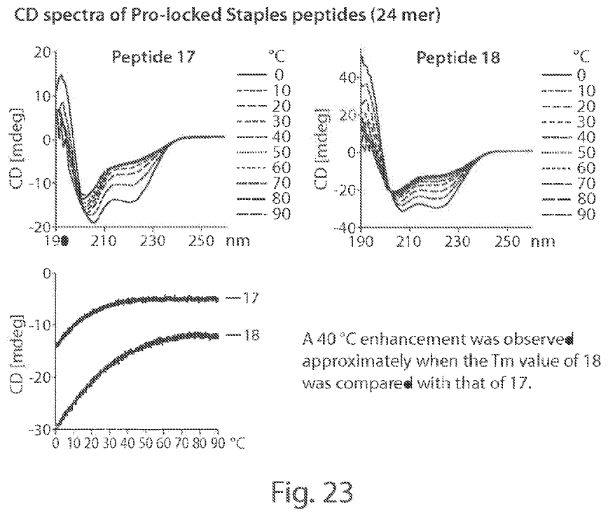

The stability of additional pro-locked peptides as determined by CD measurements is provided in FIGS. 19-23.

### Example 2: Caps for Cloaking Exposed N—H Groups in Peptides

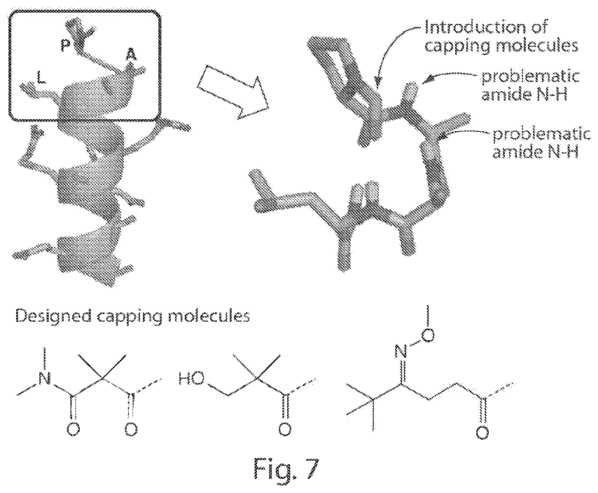

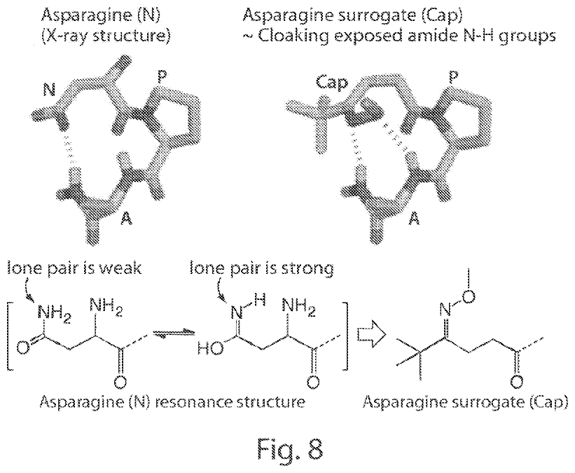

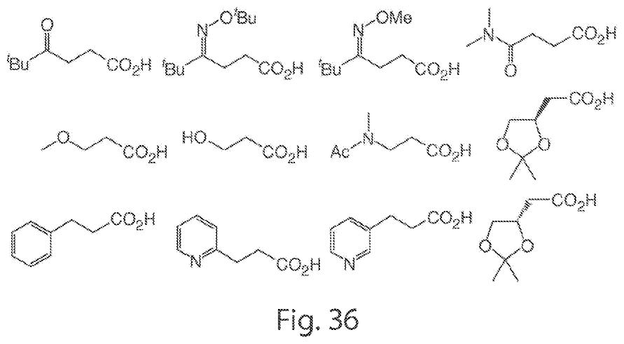

FIG. 7, FIG. 8, and FIG. 36 provide examples of peptide caps for cloaking exposed amide N—H groups.

### Example 3: Improving Passive Membrane Diffusion of Peptides

Cells were grown on chamber slides. FITC-labeled peptides 17 and 18 were added to the cell media at 0.1 microM concentration, and the cells were incubated with the peptide containing cell media. After incubation, the cells were washed and fixed. Cells were stained with DAPI, while the presence of peptide was evaluated using a confocal microscope at a wavelength appropriate for FITC.

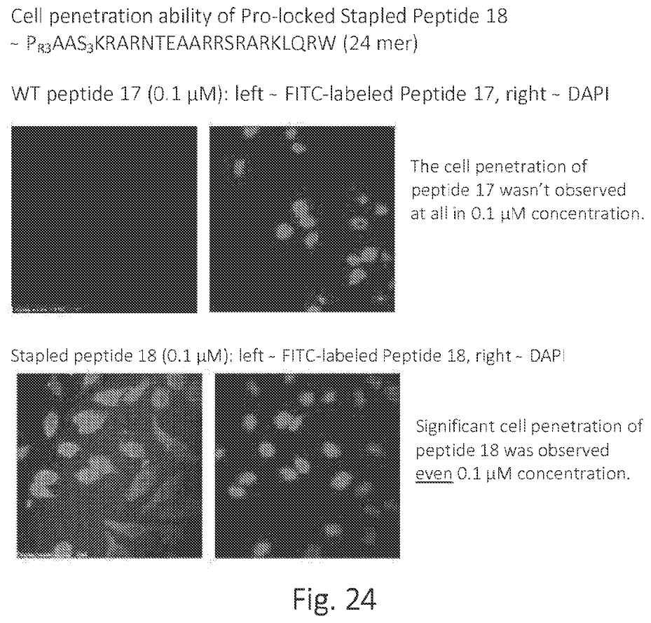

FIG. 24 shows cell penetration of Pro-locked stapled peptide 18. Significant cell penetration of a FITC-labeled Pro-locked stapled peptide 18 was shown at 0.1 microM concentration. In contrast, non-locked WT peptide 17 showed no penetration.

### Example 4: Trypsin Digestion

Peptides (10 nmole) were dissolved in 120 μl digestion buffer (0.1 M NH₄HCO₃, pH=8) and incubated with trypsin agatose for 0, 10, 20, 30, 45, 60, and 90 min. The reactions were quenched by centrifugation. The remaining substrate in the isolated supernatant was quantified by LC/MS-based peak detection at 220 nm.

### OTHER EMBODIMENTS

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1-15. (canceled)

16. A polypeptide comprising a polypeptide sequence of SEQ ID NO: 1.

17. A polypeptide comprising a polypeptide sequence of SEQ ID NO: 8.

18. A polypeptide comprising a polypeptide sequence of SEQ ID NO: 10.


SEQUENCE LISTING (58 sequences)

SEQ ID NO: 1 - protein, 14 aa, synthetic construct
PAALKRARNTEAAW

SEQ ID NO: 2 - protein, 14 aa, synthetic construct
  SITE 1: note = (R)-2-allylpyrrolidine-2-carboxylic acid
  SITE 4: note = (S)-2-amino-2-methylpent-4-enoic acid
PAAXKRARNTEAAW

SEQ ID NO: 3 - protein, 26 aa, synthetic construct
MKDPAALKRARNTEAARRSRARKLQR

SEQ ID NO: 4 - protein, 32 aa, synthetic construct
MKQLEDKVEELLSKNYHLENEVARLKKLVGER

SEQ ID NO: 5 - protein, 14 aa, synthetic construct
  SITE 1: note = (R)-2-allylpyrrolidine-2-carboxylic acid
PAALKRARNTEAAW

SEQ ID NO: 6 - protein, 14 aa, synthetic construct
  SITE 1: note = (S)-2-allylpyrrolidine-2-carboxylic acid
PAALKRARNTEAAW

SEQ ID NO: 7 - protein, 14 aa, synthetic construct
  SITE 1: note = (R)-2-allylpyrrolidine-2-carboxylic acid
  SITE 4: note = (R)-2-amino-2-methylpent-4-enoic acid
PAAXKRARNTEAAW

SEQ ID NO: 8 - protein, 14 aa, synthetic construct
  SITE 1: note = (S)-2-allylpyrrolidine-2-carboxylic acid
  SITE 4: note = (S)-2-amino-2-methylpent-4-enoic acid
PAAXKRARNTEAAW

SEQ ID NO: 9 - protein, 14 aa, synthetic construct
  SITE 1: note = (S)-2-allylpyrrolidine-2-carboxylic acid
  SITE 4: note = (R)-2-amino-2-methylpent-4-enoic acid
PAAXKRARNTEAAW

SEQ ID NO: 10 - protein, 14 aa, synthetic construct
  SITE 1: note = (2S,4R)-4-(allyloxy)pyrrolidine-2-carboxylic acid
PAALKRARNTEAAW

SEQ ID NO: 11 - protein, 14 aa, synthetic construct
  SITE 1: note = (2S,4R)-4-(allyloxy)pyrrolidine-2-carboxylic acid
  SITE 4: note = (S)-2-amino-2-methylhept-6-enoic acid
PAAXKRARNTEAAW

SEQ ID NO: 12 - protein, 14 aa, synthetic construct
  SITE 1: note = (2S,4R)-4-(allyloxy)pyrrolidine-2-carboxylic acid
  SITE 4: note = (R)-2-amino-2-methylhept-6-enoic acid
PAAXKRARNTEAAW

SEQ ID NO: 13 - protein, 14 aa, synthetic construct
  SITE 1: note = (R)-2-allylpyrrolidine-2-carboxylic acid
  SITE 4: note = (S)-2-amino-2-methylhept-6-enoic acid
PAAXKRARNTEAAW

SEQ ID NO: 14 - protein, 14 aa, synthetic construct
  SITE 1: note = (R)-2-amino-2-methylpent-4-enoic acid
  SITE 4: note = (S)-2-amino-2-methylpent-4-enoic acid
XAAXKRARNTEAAW

SEQ ID NO: 15 - protein, 8 aa, synthetic construct
PAAAAAAW

SEQ ID NO: 16 - protein, 8 aa, synthetic construct
  SITE 1: note = (R)-2-allylpyrrolidine-2-carboxylic acid
PAAAAAAW

SEQ ID NO: 17 - protein, 8 aa, synthetic construct
  SITE 1: note = (R)-2-allylpyrrolidine-2-carboxylic acid
  SITE 4: note = (S)-2-amino-2-methylpent-4-enoic acid
PAAXAAAW

SEQ ID NO: 18 - protein, 8 aa, synthetic construct
  SITE 1: note = (R)-2-allylpyrrolidine-2-carboxylic acid
  SITE 4: note = (S)-2-amino-2-methylhept-6-enoic acid
PAAXAAAW

SEQ ID NO: 19 - protein, 14 aa, synthetic construct
  SITE 1: note = (R)-2-allylpyrrolidine-2-carboxylic acid
  SITE 4: note = (S)-2-amino-2-methylpent-4-enoic acid
PAAXKRARNTEAAW

SEQ ID NO: 20 - protein, 14 aa, synthetic construct
  SITE 1: note = (R)-2-allylpyrrolidine-2-carboxylic acid
  SITE 4: note = (S)-2-amino-2-methylpent-4-enoic acid
PAAXKRARNTEAAW

SEQ ID NO: 21 - protein, 14 aa, synthetic construct
  SITE 1: note = (R)-2-amino-2-methylpent-4-enoic acid
  SITE 4: note = (S)-2-amino-2-methylpent-4-enoic acid
XAAXKRARNTEAAW

SEQ ID NO: 22 - protein, 24 aa, synthetic construct
PAALKRARNTEAARRSRARKLQRW

SEQ ID NO: 23 - protein, 24 aa, synthetic construct
  SITE 1: note = (R)-2-allylpyrrolidine-2-carboxylic acid
  SITE 4: note = (S)-2-amino-2-methylpent-4-enoic acid
PAAXKRARNTEAARRSRARKLQRW

SEQ ID NO: 24 - protein, 5 aa, synthetic construct
PAALW

SEQ ID NO: 25 - protein, 5 aa, synthetic construct
  SITE 1: note = (R)-2-allylpyrrolidine-2-carboxylic acid
  SITE 4: note = (S)-2-amino-2-methylhex-5-enoic acid
PAAXW

SEQ ID NO: 26 - protein, 14 aa, synthetic construct
  SITE 1: note = (S)-2-amino-2-methylhept-6-enoic acid, (S)-2-amino-2-methyldec-9-enoic acid, (S)-2-amino-2-methylpent-4-enoic acid or (S)-2-amino-2-methylhex-5-enoic acid
  SITE 5: note = (S)-2-amino-2-methylhept-6-enoic acid, (S)-2-amino-2-methyldec-9-enoic acid, (S)-2-amino-2-methylpent-4-enoic acid or (S)-2-amino-2-methylhex-5-enoic acid
XAALXRARNTEAAW

SEQ ID NO: 27 - protein, 6 aa, synthetic construct
NTEAAW

SEQ ID NO: 28 - protein, 6 aa, synthetic construct
  SITE 1: note = (R)-2-amino-2-methylpent-4-enoic acid
  SITE 4: note = (S)-2-amino-2-methylpent-4-enoic acid
PAAXKR

SEQ ID NO: 29 - protein, 14 aa, synthetic construct
  VARIANT 1: note = (R)-2-allylpyrrolidine-2-carboxylic acid, (S)-2-(pent-4-en-1-yl)pyrrolidine-2-carboxylic acid, (S)-2-allylpyrrolidine-2-carboxylic acid or (2S,4R)-4-(allyloxy)pyrrolidine-2-carboxylic acid
  VARIANT 7: note = (S)-2-amino-2-methylhept-6-enoic acid, (S)-2-amino-2-methyldec-9-enoic acid, (S)-2-amino-2-methylpent-4-enoic acid or (S)-2-amino-2-methylhex-5-enoic acid
XAALKRXRNTEAAW

SEQ ID NO: 30 - protein, 14 aa, synthetic construct
  VARIANT 1: note = (R)-2-allylpyrrolidine-2-carboxylic acid, (S)-2-(pent-4-en-1-yl)pyrrolidine-2-carboxylic acid, (S)-2-allylpyrrolidine-2-carboxylic acid or (2S,4R)-4-(allyloxy)pyrrolidine-2-carboxylic acid
  VARIANT 7: note = (R)-2-amino-2-methylhept-6-enoic acid, (R)-2-amino-2-methyldec-9-enoic acid or (R)-2-amino-2-methylpent-4-enoic acid
XAALKRXRNTEAAW

SEQ ID NO: 31 - protein, 14 aa, synthetic construct
  VARIANT 1: note = (R)-2-allylpyrrolidine-2-carboxylic acid, (S)-2-(pent-4-en-1-yl)pyrrolidine-2-carboxylic acid, (S)-2-allylpyrrolidine-2-carboxylic acid or (2S,4R)-4-(allyloxy)pyrrolidine-2-carboxylic acid
  VARIANT 8: note = (S)-2-amino-2-methylhept-6-enoic acid, (S)-2-amino-2-methyldec-9-enoic acid, (S)-2-amino-2-methylpent-4-enoic acid or (S)-2-amino-2-methylhex-5-enoic acid
XAALKRAXNTEAAW

SEQ ID NO: 32 - protein, 14 aa, synthetic construct
  VARIANT 1: note = (R)-2-allylpyrrolidine-2-carboxylic acid, (S)-2-(pent-4-en-1-yl)pyrrolidine-2-carboxylic acid, (S)-2-allylpyrrolidine-2-carboxylic acid or (2S,4R)-4-(allyloxy)pyrrolidine-2-carboxylic acid
  VARIANT 8: note = (R)-2-amino-2-methylhept-6-enoic acid, (R)-2-amino-2-methyldec-9-enoic acid or (R)-2-amino-2-methylpent-4-enoic acid
XAALKRAXNTEAAW

SEQ ID NO: 33 - protein, 14 aa, synthetic construct
  VARIANT 1: note = (R)-2-amino-2-methylhept-6-enoic acid, (R)-2-amino-2-methyldec-9-enoic acid or (R)-2-amino-2-methylpent-4-enoic acid
  VARIANT 8: note = (S)-2-amino-2-methylhept-6-enoic acid, (S)-2-amino-2-methyldec-9-enoic acid, (S)-2-amino-2-methylpent-4-enoic acid or (S)-2-amino-2-methylhex-5-enoic acid
XAALKRAXNTEAAW

SEQ ID NO: 34 - protein, 14 aa, synthetic construct
  VARIANT 1: note = (R)-2-allylpyrrolidine-2-carboxylic acid, (S)-2-(pent-4-en-1-yl)pyrrolidine-2-carboxylic acid, (S)-2-allylpyrrolidine-2-carboxylic acid or (2S,4R)-4-(allyloxy)pyrrolidine-2-carboxylic acid
  VARIANT 8: note = (S)-2-amino-2-methylhept-6-enoic acid, (S)-2-amino-2-methyldec-9-enoic acid, (S)-2-amino-2-methylpent-4-enoic acid or (S)-2-amino-2-methylhex-5-enoic acid
XAALKRAXNTEAAW

SEQ ID NO: 35 - protein, 14 aa, synthetic construct
  VARIANT 1: note = (R)-2-allylpyrrolidine-2-carboxylic acid, (S)-2-(pent-4-en-1-yl)pyrrolidine-2-carboxylic acid, (S)-2-allylpyrrolidine-2-carboxylic acid or (2S,4R)-4-(allyloxy)pyrrolidine-2-carboxylic acid
  VARIANT 4: note = Any synthetic amino acid
  VARIANT 7: note = (S)-2-amino-2-methylhept-6-enoic acid, (S)-2-amino-2-methyldec-9-enoic acid, (S)-2-amino-2-methylpent-4-enoic acid or (S)-2-amino-2-methylhex-5-enoic acid
XAAXKRXRNTEAAW

SEQ ID NO: 36 - protein, 14 aa, synthetic construct
  VARIANT 1: note = (R)-2-allylpyrrolidine-2-carboxylic acid, (S)-2-(pent-4-en-1-yl)pyrrolidine-2-carboxylic acid, (S)-2-allylpyrrolidine-2-carboxylic acid or (2S,4R)-4-(allyloxy)pyrrolidine-2-carboxylic acid
  VARIANT 4: note = Any synthetic amino acid
  VARIANT 8: note = (R)-2-amino-2-methylhept-6-enoic acid, (R)-2-amino-2-methyldec-9-enoic acid or (R)-2-amino-2-methylpent-4-enoic acid
XAAXKRAXNTEAAW

SEQ ID NO: 37 - protein, 14 aa, synthetic construct
  VARIANT 1: note = (R)-2-allylpyrrolidine-2-carboxylic acid, (S)-2-(pent-4-en-1-yl)pyrrolidine-2-carboxylic acid, (S)-2-allylpyrrolidine-2-carboxylic acid or (2S,4R)-4-(allyloxy)pyrrolidine-2-carboxylic acid
  VARIANT 4: note = Any synthetic amino acid
  VARIANT 11: note = (S)-2-amino-2-methylhept-6-enoic acid, (S)-2-amino-2-methyldec-9-enoic acid, (S)-2-amino-2-methylpent-4-enoic acid or (S)-2-amino-2-methylhex-5-enoic acid
XAAXKRARNTXAAW

SEQ ID NO: 38 - protein, 14 aa, synthetic construct
  VARIANT 1: note = (R)-2-allylpyrrolidine-2-carboxylic acid, (S)-2-(pent-4-en-1-yl)pyrrolidine-2-carboxylic acid, (S)-2-allylpyrrolidine-2-carboxylic acid or (2S,4R)-4-(allyloxy)pyrrolidine-2-carboxylic acid
  VARIANT 4: note = (S)-2-amino-2-methylhept-6-enoic acid, (S)-2-amino-2-methyldec-9-enoic acid, (S)-2-amino-2-methylpent-4-enoic acid or (S)-2-amino-2-methylhex-5-enoic acid
  VARIANT 12: note = (R)-2-amino-2-methylhept-6-enoic acid, (R)-2-amino-2-methyldec-9-enoic acid or (R)-2-amino-2-methylpent-4-enoic acid
XAAXKRARNTEXAW

SEQ ID NO: 39 - protein, 5 aa, synthetic construct
DPAAL

SEQ ID NO: 40 - protein, 14 aa, synthetic construct
  SITE 1: note = (R)-2-allylpyrrolidine-2-carboxylic acid
  SITE 4: note = (S)-2-amino-2-methylhex-5-enoic acid
XAAXKRARNTEAAW

SEQ ID NO: 41 - protein, 21 aa, synthetic construct
  SITE 1: note = (R)-2-allylpyrrolidine-2-carboxylic acid
  SITE 4: note = (S)-2-amino-2-methylhept-6-enoic acid
XAAXKRARNTERSRARKLQRW

SEQ ID NO: 42 - protein, 14 aa, synthetic construct
  SITE 1: note = (R)-2-amino-2-methylhept-6-enoic acid
  SITE 4: note = (S)-2-amino-2-methylhept-6-enoic acid
XAAXKRARNTEAAW

SEQ ID NO: 43 - protein, 14 aa, synthetic construct
  SITE 1: note = (S)-2-amino-2-methylhept-6-enoic acid
  SITE 5: note = (S)-2-amino-2-methylhept-6-enoic acid
XAALXRARNTEAAW

SEQ ID NO: 44 - protein, 14 aa, synthetic construct
  SITE 1: note = (S)-2-(pent-4-en-1-yl)pyrrolidine-2-carboxylic acid
  SITE 7: note = (S)-2-amino-2-methyldec-9-enoic acid
PAALKRXRNTEAAW

SEQ ID NO: 45 - protein, 14 aa, synthetic construct
  SITE 1: note = (S)-2-(pent-4-en-1-yl)pyrrolidine-2-carboxylic acid
  SITE 7: note = (R)-2-amino-2-methyldec-9-enoic acid
PAALKRXRNTEAAW

SEQ ID NO: 46 - protein, 14 aa, synthetic construct
  SITE 1: note = (S)-2-(pent-4-en-1-yl)pyrrolidine-2-carboxylic acid
  SITE 8: note = (S)-2-amino-2-methyldec-9-enoic acid
PAALKRAXNTEAAW

SEQ ID NO: 47 - protein, 14 aa, synthetic construct
  SITE 1: note = (S)-2-(pent-4-en-1-yl)pyrrolidine-2-carboxylic acid
  SITE 8: note = (R)-2-amino-2-methyldec-9-enoic acid
PAALKRAXNTEAAW

SEQ ID NO: 48 - protein, 14 aa, synthetic construct
  SITE 1: note = (R)-2-amino-2-methylhept-6-enoic acid
  SITE 8: note = (S)-2-amino-2-methyldec-9-enoic acid
PAALKRAXNTEAAW

SEQ ID NO: 49 - protein, 14 aa, synthetic construct
  SITE 1: note = (R)-2-allylpyrrolidine-2-carboxylic acid
  SITE 4: note = 2-amino-2-(pent-4-en-1-yl)hept-6-enoic acid
  SITE 7: note = (S)-2-amino-2-methylhept-6-enoic acid
PAAXKRXRNTEAAW

SEQ ID NO: 50 - protein, 14 aa, synthetic construct
  SITE 1: note = (R)-2-allylpyrrolidine-2-carboxylic acid
  SITE 4: note = 2-amino-2-(pent-4-en-1-yl)hept-6-enoic acid
  SITE 8: note = (R)-2-amino-2-methylhept-6-enoic acid
PAAXKRAXNTEAAW

SEQ ID NO: 51 - protein, 14 aa, synthetic construct
  SITE 1: note = (R)-2-allylpyrrolidine-2-carboxylic acid
  SITE 4: note = 2-amino-2-(pent-4-en-1-yl)hept-6-enoic acid
  SITE 11: note = (S)-2-amino-2-methyldec-9-enoic acid
PAAXKRARNTXAAW

SEQ ID NO: 52 - protein, 14 aa, synthetic construct
  SITE 1: note = Acetylated (R)-2-allylpyrrolidine-2-carboxylic acid
  SITE 4: note = (S)-2-amino-2-methylpent-4-enoic acid
  SITE 12: note = (R)-2-amino-2-methylpent-4-enoic acid
  SITE 14: note = Tryptophan NH-Allyl
PAAXKRARNTEXAW

SEQ ID NO: 53 - protein, 14 aa, synthetic construct
  SITE 1: note = Acetylated (R)-2-allylpyrrolidine-2-carboxylic acid
  SITE 4: note = (S)-2-amino-2-methylpent-4-enoic acid
  SITE 12: note = (R)-2-amino-2-methylhex-5-enoic acid
  SITE 14: note = Tryptophan NH-Allyl
PAAXKRARNTEXAW

SEQ ID NO: 54 - protein, 14 aa, synthetic construct
  SITE 1: note = Acetylated (R)-2-allylpyrrolidine-2-carboxylic acid
  SITE 12: note = (R)-2-amino-2-methylhept-6-enoic acid
  SITE 14: note = Tryptophan NH-Allyl
PAASKRARNTEXAW

SEQ ID NO: 55 - protein, 14 aa, synthetic construct
  SITE 1: note = Acetylated (R)-2-allylpyrrolidine-2-carboxylic acid
  SITE 4: note = (S)-2-amino-2-methylpent-4-enoic acid
  SITE 12: note = (R)-2-amino-2-methylpent-4-enoic acid
  SITE 14: note = Tryptophan NH-Butenyl
PAAXKRARNTEXAW

SEQ ID NO: 56 - protein, 14 aa, synthetic construct
  SITE 1: note = Acetylated (R)-2-allylpyrrolidine-2-carboxylic acid
  SITE 4: note = (S)-2-amino-2-methylpent-4-enoic acid
  SITE 12: note = (R)-2-amino-2-methylhex-5-enoic acid
  SITE 14: note = Tryptophan NH-Butenyl
PAAXKRARNTEXAW

SEQ ID NO: 57 - protein, 14 aa, synthetic construct
  SITE 1: note = Acetylated (R)-2-allylpyrrolidine-2-carboxylic acid
  SITE 4: note = (S)-2-amino-2-methylpent-4-enoic acid
  SITE 12: note = (R)-2-amino-2-methylhept-6-enoic acid
  SITE 14: note = Tryptophan NH-Butenyl
PAAXKRARNTEXAW

SEQ ID NO: 58 - protein, 5 aa, synthetic construct
  SITE 1: note = (R)-2-allylpyrrolidine-2-carboxylic acid
  SITE 4: note = (S)-2-amino-2-methylpent-4-enoic acid
PAAXW